(19) United States
(12) Patent Application Publication
(10) Pub. No.: US 2023/0355918 A1
(43) Pub. Date: Nov. 9, 2023

(54) METHOD AND SYSTEM FOR IMPROVING A PHYSIOLOGICAL CONDITION OF A SUBJECT

(71) Applicant: LIQUID OXIGEN (LOX) B.V., Amsterdam (NL)

(72) Inventors: Paulus Oomen, Amsterdam (NL); Rona Geffen, Amsterdam (NL)

(21) Appl. No.: 18/021,680

(22) PCT Filed: Aug. 18, 2021
(86) PCT No.: PCT/NL2021/050514

§ 371 Date: Feb. 16, 2023

(30) Foreign Application Priority Data
Aug. 19, 2020 (NL) 2026299

(51) Int. Cl. A61M 21/02; H04S 7/00

(52) U.S. Cl. CPC A61M 21/02; H04S 7/302; H04S 2400/11; A61M 2021/0027

(57) ABSTRACT

A method for improving a physiological condition of a subject, e.g. a human or animal, is disclosed. The method comprises providing an audio signal to the subject, wherein the audio signal is associated with a virtual sound source having a shape and a position relative to the subject. The virtual sound source is defined by a plurality of virtual points, each virtual point having a position relative to the subject. The audio signal comprises audio signal components for the respective virtual points of the virtual sound source, wherein each audio signal component has been determined based on the virtual position of its associated virtual point such that the audio signal is perceived by the subject as originating from the virtual sound source having said shape and said position relative to the subject. Further, a system for performing this method is also disclosed.

### FIELD OF THE INVENTION

This disclosure relates to methods and systems for improving a physiological condition of a subject, such as a human or animal. In particular to such methods wherein an audio signal is configured such that it is perceived by the subject as originating from a virtual source having a position and a shape. This disclosure further relates to systems for providing such an audio signal to a subject.

### BACKGROUND

Homeostasis refers to a self-regulating process by which biological systems tend to maintain stability while adjusting to conditions that are optimal for survival. If homeostasis is successful, life continues; if unsuccessful, disaster or death ensues. The stability attained is a dynamic equilibrium, in which continuous change occurs yet relatively uniform conditions prevail (Encyclopaedia Britannica, 2018). Homeostasis is the ability to maintain a constant internal environment in response to environmental changes. It is a unifying principle of biology. The nervous and endocrine systems control homeostasis in the body through feedback mechanisms involving various organs and organ systems (R Bailey, 2017).

Various methods exist that are known to improve the physiological state of a person, such as meditation and mindfulness practices (M Goyal, JAMA Intern Med 2014 March; 174(3):357-68), music therapy (H S Shin et al., Asian Nursing Research, Volume 5, Issue 1, March 2011, Pages 19-27), physical activity such as yoga (C Woodyard, Int J Yoga. 2011 July-December; 4(2): 49-54) and dance therapy (Y Ja Jeong et al. 2009 International Journal of Neuroscience, Volume 115, 2005—Issue 12 Pages 1711-1720), and treatment of the body, such as massage (T Field, Complement Ther Clin Pract. 2016 August; 24: 19-31) and sauna (J A. Laukkanen et al, Mayo Clinic Proceedings, Volume 93, ISSUE 8, P1111-1121, Aug. 1, 2018) to medicinal substances such as tranquilizers (K A. Holroyd et al, JAMA. 2001; 285(17):2208-2215) and medicinal herbs (D R Wilson, 2019).

A disadvantage of these methods is that they require a person to invest significant effort and/or time to improve his or her physiological state and/or that they cause negative side effects. The latter disadvantage typically arises when medicines are used. Therefore there is a need in the art for methods and systems that enable someone to improve his or her physiological state that require less time and/or effort and/or do not cause negative side-effects.

### SUMMARY

Therefore a method for improving a physiological condition of a subject, e.g. a human or animal, is disclosed. The method comprises providing an audio signal to the subject, wherein the audio signal is associated with a virtual sound source having a shape and a position relative to the subject. The virtual sound source is defined by a plurality of virtual points, each virtual point having a position relative to the subject. The audio signal comprises audio signal components for the respective virtual points of the virtual sound source, wherein each audio signal component has been determined based on the virtual position of its associated virtual point such that the audio signal is perceived by the subject as originating from the virtual sound source having said shape and said position relative to the subject.

The audio signal may be configured such that it is perceived by the subject to originate from a virtual sound source that is positioned at a depth below the subject or at a height above the subject and/or at a distance, e.g. a horizontal distance, from the subject.

The inventor has found out that providing such an audio signal to a subject improves the subject's physiological condition, e.g. improves homeostasis of the human body, which refers to the tendency of the body towards a stable equilibrium between its interdependent elements, the effects thereof associated with feelings of increased mental and physical wellbeing by the subject. The method may be understood to reduce the stress as experienced by the subject and/or cause the subject to feel more relaxed and/or cause a pleasant sensation for the subject. This disclosure thus offers an effective method to improve the physiological condition that is faster than the methods known-in-the-art. The method may yield beneficial results within less than 5 minutes. Further, the method does not inflict drowsiness or tiredness and could thus be very beneficial to students, working population and stay-at-home parents with short amounts of time before they need to get back to perform tasks at a high level.

Providing the audio signal to the subject may also be referred to herein as projecting the virtual sound source.

As said, the subject exposed to such an audio signal may experience a change in physiological state after a short time period of exposure, that is, after less than 5 minutes. The improved physiological state of the subject achieved by the methods described herein, can be determined based on a measured decrease in power ratio, i.e. mean difference slope, between the Alpha-band and other frequency bands of the brain activity (Delta, Theta, Beta, Gamma); in particular, a significant decrease in Alpha mean power and a significant decrease in Alpha:Beta power ratio. A decrease of this ratio is indicative of an improved physiological state of the subject. Additionally or alternatively, the improved physiological state of the subject can be determined based on a significant decrease in the Low-Frequency (LF):High-Frequency (HF) power ratio of the Heart Rate Variability (HRV). A decrease of this ratio is indicative of an improved physiological state of the subject. Additionally or alternatively, the improved physiological state can be determined based on effects of increased relaxation, improved emotional balance and enhanced state of mental clarity as reported by subjects, the results of which are confirmed by data obtained through research.

The physiological effects of such audio signal are claimed on the basis of data obtained from 50 participating subjects, who answered questionnaires pre- and post-exposure to the audio signal and were monitored on Brain Activity (EEG) and Heart-Rate Variability (HRV) showing the effects of the method compared to base-state of the subjects. Of the participating subjects, a test group was provided with a “standard” audio signal, i.e. an audio signal that a user does not perceive as originating from a virtual sound source having a certain shape and position, to obtain reference measurements. For all subjects, the same loudspeakers were used to provide the audio signal to the subject.

The data obtained through research show significant results of the physiological effects associated with improved physiological states, e.g. improved homeostasis, in response to an audio signal that is configured such that it is perceived by the subject as originating from a virtual sound source having a position and a shape. The effects can be considered novel, as it was not known prior to the invention that a method for sound projection could generate a marked change in the brain activity and vital signs indicating an improved physiological state; and, that such physiological changes in the human body are not achieved with a standard audio signal. Thus, an effect can be distinguished that can be attributed to the audio signal being configured as it is, compared to other commonly known and described attributes of sound, such as its pitch, loudness, timbre, etc.

The method is effortless for the subject, i.e. no prior instructions, training practice or specific skills are required of the subject and is thus attainable for a broader group of people.

The method does not require obtaining physiological data of the subject prior to use or measured in real-time, thus simplifying required technical infrastructure and allowing straight-forward and passive user application.

The method is physically non-invasive as no intake of substance by the subject is required and there are no negative side-effects present.

The method is socially non-invasive as it can be done in private, it does not require physical contact with a specialist or removal of clothes and/or otherwise actions by subjects that may be considered compromising.

Furthermore, the method is very suitable for people with short communication and sound resistance such as people suffering from various head trauma and comastoisis conditions. These populations can, due to their condition, only be exposed to sound for very short periods of time (20 minutes or less). In addition, these populations, such as other severely injured populations suffering from acute physical conditions, cannot engage in many of the other existing methods due to their mobility disability or consciousness deprivation. The method is also highly suitable for people with a short patience span such as children dealing with ADHD or cognitive conditions.

The audio signal can advantageously be provided using existing audio reproduction formats and existing industry standards.

Thus, the methods described herein may help to more effectively improve the physiological condition of a subject, which may for example improve productivity of work and study places, and help to reduce fear and aggression in society, and the resulting expressions thereof. The methods can also be beneficially used in home sound booths, spa and wellness centers and as a mediation aid.

Preferably, the method is a non-therapeutic method. This may be understood as that the purpose of the method is not to restore an organism from a pathological to its original condition, or to prevent pathology in the first place, but to improve the performance of an organism taking as its starting point a normal, healthy state.

It should be appreciated that the method steps of the embodiments described herein may be computer-implemented.

In an embodiment, the audio signal is obtainable by
obtaining virtual sound source information defining the respective positions of the virtual points relative to the subject, the virtual points defining the virtual sound source having said shape and said position relative to the subject, and
obtaining an input audio signal, and
determining the respective audio signal components for the respective virtual points based on the input audio signal and based on the respective positions of the virtual points, wherein for each audio signal component respectively associated with a virtual point, determining the audio signal component comprises
modifying the input audio signal to obtain a modified audio signal component using a signal delay operation introducing a time delay, wherein the time delay is based on the defined position of the virtual point associated with the audio signal component; and
determining the audio signal component based on a combination, e.g. a summation, of the input audio signal, or of an inverted and/or attenuated or amplified version of the input audio signal, and the modified audio signal component, and
combining the determined audio signal components to obtain the audio signal.

This embodiment uses an audio signal that can be easily determined based on the virtual sound source as defined by the plurality of virtual points and an input audio signal.

In this embodiment, preferably, the time delay that is introduced by the signal delay operation for the determination of the audio signal component in question is based on the virtual position of the virtual point associated with the audio signal component in question, in particular based on the virtual position of this virtual point relative to the dimensional shape of the virtual sound source.

The positions of the virtual points defined by the virtual sound source information are preferably defined with respect to each other and with respect to the subject.

It should be appreciated that the method for improving the physiological condition of a subject may comprise determining the audio signal based on an input audio signal and virtual sound source information defining the virtual sound source, for example defining the shape of the virtual sound source and its position with respect to the subject. Such determination of the audio signal may comprise any of the steps described herein that result in obtaining the audio signal.

In an embodiment, the input audio signal is an audio signal produced by a tuning fork, preferably by an unweighted tuning fork.

In principle, the input audio signal used for generating the audio signal that is to be provided to the subject can be any audio signal.

In an embodiment, the method comprises providing the audio signal to the subject using a plurality of loudspeakers. This embodiment further comprises determining a loudspeaker audio signal for each loudspeaker, wherein each loudspeaker audio signal is determined based on the plurality of audio signal components, and providing the loudspeaker audio signals to the respective loudspeakers.

This embodiment provides a convenient manner of distributing the audio signal over a plurality of loudspeakers. Such distribution may also be referred to as panning.

In an embodiment, determining a loudspeaker audio signal for each loudspeaker comprises, for each loudspeaker audio signal, attenuating each audio signal component based on a loudspeaker specific coefficient in order to obtain a loudspeaker specific set of attenuated audio signal components and combining, e.g. summing, the attenuated audio signal components in the loudspeaker specific set of attenuated audio signal components.

It should be appreciated that loudspeaker specific may be understood as that each loudspeaker is associated with its own loudspeaker specific coefficient. The different loudspeaker coefficients for the different loudspeakers are not necessarily all different from each other, some of these coefficients, or even all coefficients, may have the same value. Further, loudspeaker specific set may be understood as that each loudspeaker has its own set of attenuated audio signal components. The different sets of loudspeaker specific components for the different loudspeakers are not necessarily all different from each other.

In such embodiment, the loudspeaker coefficient for a loudspeaker may be determined based on the position of the loudspeaker in question relative to the subject. In such embodiment, the subject preferably has a predetermined position with respect to each of the loudspeakers.

In an embodiment, the virtual sound source is shaped as a cube or pyramid or sphere. These shapes effectively improve the physiological condition of the subject. It should be appreciated that the virtual sound source can have any shape or form.

In an embodiment, the audio signal is configured such that it is perceived by the subject that said virtual sound source is surrounding the subject. In this embodiment, in other words, the virtual sound source is surrounding the subject.

In an embodiment, the method comprises providing the audio signal to the subject using a plurality of loudspeakers that surround the subject.

The plurality of loudspeakers may comprise a loudspeaker in front of the subject and a loudspeaker behind the subject. Additionally or alternatively, the plurality of loudspeakers comprises a loudspeaker to the right of the subject and a loudspeaker to the left of the subject. Additionally or alternatively, the plurality of loudspeakers comprises a loudspeaker above the subject and a loudspeaker below the subject.

For example, in an embodiment, the plurality of loudspeakers comprises at least eight loudspeakers:
a loudspeaker above the subject;
a loudspeaker in front of, below the subject;
a loudspeaker in front of, to the left of, above the subject;
a loudspeaker in front of, to the right of, above the subject;
a loudspeaker behind, above the subject;
a loudspeaker behind, to the left of, below the subject;
a loudspeaker behind, to the right of, below the subject;
a loudspeaker below the subject.

The plurality of loudspeakers may be positioned respectively at equal distance from the subject. Additionally or alternatively, the plurality of loudspeakers may be positioned equidistant from each other.

In an embodiment, the audio signal is provided to the subject for at least 1 minute, preferably for at least 2 minutes, more preferably for at least 5 minutes. The inventor has found out that the improvements to the physiological condition of the subject can be achieved quickly, even within 5 minutes.

In an embodiment, the virtual sound source associated with the audio signal changes shape and/or position while the audio signal is provided to the subject thus wherein the respective positions relative to the subject of the respective virtual points defining the virtual sound source change while the audio signal is provided to the subject such that the audio signal is perceived by the subject as originating from the virtual sound source having a varying position and/or orientation relative to the subject.

Thus, in this embodiment, the subject perceives the audio signal as originating from a virtual sound source that moves and/or changes shape. The inventor has found out that such moving and/or changing virtual sound source may also benefit the physiological condition of the subject.

In this embodiment, the virtual points may be understood to move with respect to subject if the virtual sound source moves with respect to the subject. Further, virtual points may be understood to move with respect to each other if the virtual sound source changes shape.

In an embodiment, one or more virtual points of the virtual sound source are virtually positioned at a depth below the subject. Then, the audio signal is obtainable by
for each audio signal component associated with a virtual point that is positioned at a virtual depth below the subject, adding depth characteristics to the audio signal component in question comprising modifying the audio signal component in question using a time delay operation introducing a time delay, a signal attenuation and a signal feedback operation in order to obtain a modified version of the audio signal component and combining the modified version of the audio signal component with the audio signal component in question, wherein
the signal attenuation is performed in dependence of the virtual depth below the subject of the virtual point associated with the audio signal component in question.

It should be appreciated that a depth input signal may be an audio signal component associated with a virtual point and that the depth output signal is the same audio signal component with depth information added to it. Said signal attenuation may then be performed in dependence of the depth of the virtual point associated with the audio signal component in question below the subject.

In an embodiment, one or more virtual points of the virtual sound source are virtually positioned at a height above the subject, wherein the audio signal is obtainable by
for each audio signal component associated with a virtual point that is positioned at a virtual height above the subject, adding height characteristics to the audio signal component in question comprising modifying the audio signal component in question using a signal inverting operation, a signal delay operation introducing a time delay and a signal attenuation to obtain a modified version of the audio signal component and combining the modified version of the audio signal component with the audio signal component in question, wherein
the signal attenuation is performed in dependence of the virtual height of the virtual sound source.

It should be appreciated that the height input signal may be an audio signal component associated with a virtual point and that the height output signal is the same audio signal component with height information added to it. Said signal attenuation may then be performed in dependence of the height above the subject of the virtual point associated with the audio signal component in question.

In an embodiment, one or more virtual points of the virtual sound source are virtually positioned at a virtual distance from the subject, wherein the audio signal is obtainable by
for each audio signal component associated with a virtual point that is positioned at a virtual distance from the subject, adding distance characteristics to the audio signal component in question comprising modifying the audio signal component in question using a first signal delay operation introducing a first time delay, a first signal attenuation operation and a signal feedback operation in order to obtain a first modified version of the audio signal component and combining the first modified version of the audio signal component with the audio signal component in question to obtain a second modified version of the audio signal component and performing a second signal attenuation and optionally a second signal delay operation introducing a second time delay on the second modified version of the audio signal component, wherein
the first and second signal attenuation are performed in dependence of the virtual distance from the subject.

Said first and second signal attenuations may then be performed in dependence of the distance between the subject and the virtual point associated with the audio signal component in question.

This disclosure further relates to a system for improving a physiological condition of a subject, e.g. a human or animal. The system comprises a data processing system for determining, based on an input audio signal, an audio signal that is configured such that it is perceived by the subject as originating from a virtual sound source having a shape and optionally a position. The system further comprises one or more loudspeakers for providing the determined audio signal to the subject.

One aspect of this disclosure relates to a computer comprising a computer readable storage medium having computer readable program code embodied therewith, and a processor, preferably a microprocessor, coupled to the computer readable storage medium, wherein responsive to executing the computer readable program code, the processor is configured to perform the method according any of the embodiments described herein.

One aspect of this disclosure relates to a computer program or suite of computer programs comprising at least one software code portion or a computer program product storing at least one software code portion, the software code portion, when run on a computer system, being configured for executing the method according to any of the embodiments described herein.

One aspect of this disclosure relates to a non-transitory computer-readable storage medium storing at least one software code portion, the software code portion, when executed or processed by a computer, is configured to perform the method according to any of the embodiments described herein.

One aspect of this disclosure relates to a computer program comprising instructions to cause any of the systems for improving the physiological condition of a subject described herein, to execute the method according to any of the embodiments described herein.

As will be appreciated by one skilled in the art, aspects of the present invention may be embodied as a system, a method or a computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a processor/microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present invention, a computer readable storage medium may be any tangible medium that can contain, or store, a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or a central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

Moreover, a computer program for carrying out the methods described herein, as well as a non-transitory computer readable storage-medium storing the computer program are provided. A computer program may, for example, be downloaded (updated) to the existing systems, e.g. optical receivers, remote controls, smartphones, or tablet computers, or be stored upon manufacturing of these systems.

Elements and aspects discussed for or in relation with a particular embodiment may be suitably combined with elements and aspects of other embodiments, unless explicitly stated otherwise.

Embodiments of the present invention will be further illustrated with reference to the attached drawings, which schematically will show embodiments according to the invention. It will be understood that the present invention is not in any way restricted to these specific embodiments.

### BRIEF DESCRIPTION OF THE DRAWINGS

Aspects of the invention will be explained in greater detail by reference to exemplary embodiments shown in the drawings, in which:

FIG. 1A is a flow chart illustrating a method and system according to embodiments;

FIG. 1B schematically depicts a virtual sound source that is shaped as a pyramid and surrounds the subject;

FIGS. 2A, 2B, 2C illustrate an embodiment;

FIG. 3 illustrates an embodiment wherein digital signal processing is implemented;

FIG. 4A illustrates how the virtual sound source can be defined according to an embodiment;

FIGS. 4B-4D illustrate examples of grids that may be used to define a virtual sound source according to an embodiment;

FIGS. 4E-4T illustrate examples of virtual sound sources;

FIG. 5A schematically shows a loudspeaker configuration according to an embodiment;

FIGS. 5B, 5C and 5D illustrate the virtual sound sources as used in conducted experiments;

FIG. 5E illustrates a “standard” stereo projection used as a reference sound signal in conducted experiments and illustrates the signal process that was used to obtain the reference sound signal during conducted experiments;

FIG. 5F illustrates the signal process that was used to obtain the virtual sound sources during conducted experiments;

FIG. 6 is a flow chart illustrating how the loudspeaker audio signals can be determined;

FIG. 7 illustrates how the audio signal components respectively associated with the virtual points can be determined according to an embodiment;

FIGS. 8A-C illustrate how shape characteristics can be added to an audio signal component according to an embodiment;

FIGS. 9A-C illustrate how depth characteristics can be added to an audio signal component according to an embodiment;

FIGS. 10A-C illustrate how height characteristics can be added to an audio signal component according to an embodiment;

FIGS. 11A-C illustrates how distance characteristics can be added to an audio signal component according to an embodiment;

FIG. 12 illustrates in detail how the audio signal can be determined according to an embodiment;

FIGS. 13A-C illustrate how the audio signal can be determined according to an embodiment;

FIG. 14A shows the audio signal of an unweighted tuning fork recorded in a sound recording studio, which may serve as input audio signal according to an embodiment;

FIG. 14B shows a spectrograph of an unweighted tuning fork;

FIG. 15A shows a mean spectrograph of a recorded unweighted tuning fork, which was used as a reference audio signal during conducted experiments;

FIG. 15B shows a mean spectrograph of an audio signal according to an embodiment that has been generated with a recorded unweighted tuning fork as input audio signal and that projects a pyramid shaped virtual sound source;

FIG. 15C. shows a mean spectrograph of an audio signal according to an embodiment that has been generated with a recorded unweighted tuning fork as input audio signal and that projects a cube shaped virtual sound source;

FIG. 15D shows a mean spectrograph of an audio signal according to an embodiment that has been generated with a recorded unweighted tuning fork as input audio signal and that projects a spherical virtual sound source;

FIG. 16A shows measured physiological effects in test subjects after having been provided a reference audio signal;

FIG. 16B shows measured physiological effects in test subjects after having been provided an audio signal according to an embodiment wherein the audio signal projects a pyramid shaped virtual sound source;

FIG. 16C shows measured physiological effects in test subjects after having been provided an audio signal according to an embodiment wherein the audio signal projects a cube shaped virtual sound source;

FIG. 16D shows measured physiological effects in test subjects after having been provided an audio signal according to an embodiment wherein the audio signal projects a spherical virtual sound source;

FIG. 17 summarizes results relating to brain activity for the different experiments that were conducted;

FIG. 18 shows experimental results relating to the Alpha:Beta power ratio;

FIG. 19 shows experimental results relating to the LF:HF power ratio of the Heart Rate Variability;

FIG. 20A shows a summary of the results from MDQM questionnaires answered by the subjects pre- and post-exposure to the sound stimuli;

FIG. 20B shows a larger report resulting from MDQM questionnaires answered by the subjects pre- and post-exposure to the sound stimuli;

FIGS. 21A-E illustrate a system according to an embodiment;

FIGS. 22A-B illustrate a system according to an embodiment;

FIG. 23 illustrates a data processing system according to an embodiment.

### DETAILED DESCRIPTION OF THE DRAWINGS

In the figures, identical reference numerals refer to identical or similar elements. Further, a flow chart may be understood to depict both an embodiment of a method in that several steps are depicted as well as an embodiment of a system, such as a circuit, that is configured to process signals as depicted in the flow chart. Further, elements that are indicted by dashed lines are optional elements.

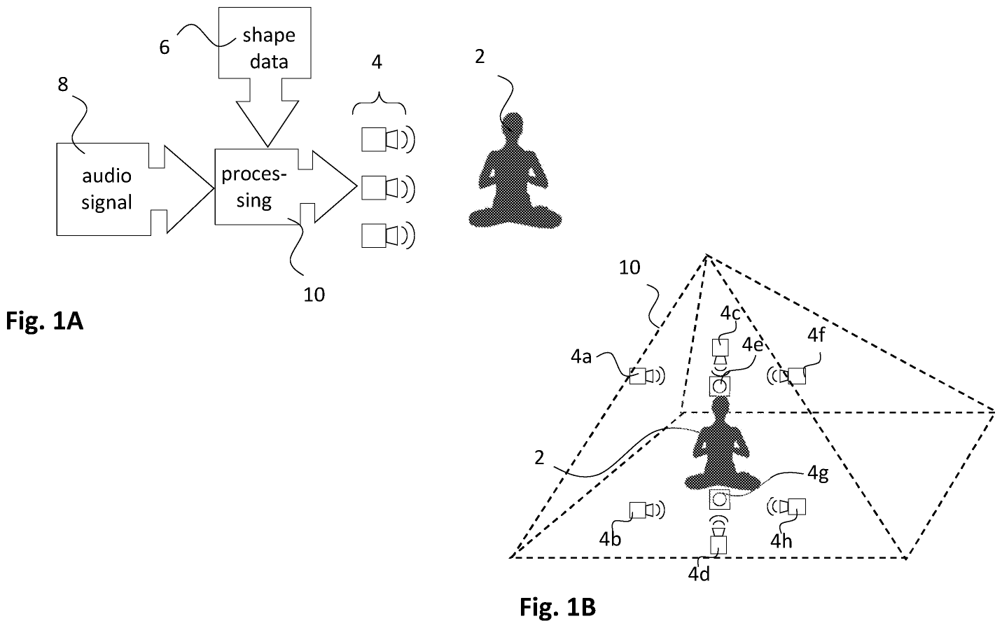

FIG. 1A is a flow chart illustrating a method for improving a physiological condition, e.g. the homeostasis, of a subject. In the depicted embodiment, one (or more) audio signal(s) 8 and virtual sound source information 6 are input for a method for determining an audio signal that can be used to improve the physiological condition of a subject 2. The virtual sound source information 6 in this embodiment defines a plurality of virtual points, wherein each virtual point has a virtual position with respect to other virtual points so that the virtual points together define a shape of the virtual sound source. The virtual sound source information 6 may, additionally, define the virtual positions with respect to the subject 2 so that the virtual sound source has a certain position with respect to the subject 2. The virtual sound source may for example be positioned above or below the subject 2 and/or be positioned at a certain horizontal distance from the subject 2, e.g. a couple of meters in front of the subject 2. In principle, the distance, height or depth can be infinitely large and is not limited by the physical configuration of the loudspeakers. The virtual sound source information 6 is used to modify the audio input signal(s) 8 and obtain an audio signal. The audio signal comprises audio signal components for the respective virtual points such that the audio signal is perceived by the subject as originating from the virtual sound source having the shape and position as defined by the virtual points. Then, the audio signal, in the depicted embodiment, is distributed to one (or more) loudspeaker(s) 4. The resulting projection of a virtual sound source with a distinct shape and position induces improved physiological condition of the subject 2.

The method 10 for determining the audio signal may comprise
obtaining virtual sound source information 6 defining the respective positions of the virtual points relative to the dimensional shape of the virtual sound source and relative to the subject 2, the virtual points defining the virtual sound source having said shape and said position relative to the subject 2, and
obtaining an input audio signal 8, and
determining the respective audio signal components for the respective virtual points based on the input audio signal 8 and based on the respective positions of the virtual points, wherein
for each audio signal component respectively associated with a virtual point, determining the audio signal component comprises
   modifying the input audio signal 8 to obtain a modified audio signal component using a signal delay operation introducing a time delay, wherein the time delay is based on the defined position of the virtual point associated with the audio signal component relative to the dimensional shape of the virtual sound source; and
   determining the audio signal component based on a combination, e.g. a summation, of the input audio signal 8, or of an inverted and/or attenuated or amplified version of the input audio signal 8, and the modified audio signal component, and
   combining the determined audio signal components to obtain the audio signal.

The method referred to herein provides an accessible and efficient way to improve the physiological condition, e.g. to improve the homeostasis, of a subject 2, by means of encoding virtual source information 6 into sound waves propagating from a sound output medium, e.g. loudspeakers 4. It should be understood that the claims of the described improved physiological effects may be considered valid with the whole of the described methods used; and/or any separate part of the described methods used to achieve such effects; and/or any other methods used to obtain an audio signal that is perceived by a subject to originate from a virtual sound source having a shape, be it prior-art methods or future to-be-invented methods. The methods described herein for determining and/or generating the audio signal may include digital processing of sound signals, analogue circuits to modify sound signals and/or in combination with methods of acoustic modification and generation of sound to obtain sound projection of a defined dimensional shape, size and density.

FIG. 1B shows a subject 2 positioned in the middle of a loudspeaker configuration comprising loudspeakers 4a-4h that play back the audio signal as described herein that provides for projection of a virtual sound source 10 with a distinct shape and position. The resulting physiological response of the subject 2 to the virtual sound source projection indicates an improved homeostasis.

In an embodiment, the virtual sound source 10 is shaped as a pyramid as depicted. It should be understood that the method is not limited to one type of shape, and claimed effects comprise the encoding of shape in an audio signal, as distinct from other commonly described attributes of sound such as its pitch, loudness, timbre etc; and, that embodiments may include any type and/or combination of shape and spatial transformation of such shape.

In an embodiment, the loudspeakers 4 may be placed surrounding the subject 2 vertically and horizontally, i.e. surrounding the subject equally from above, below, front, back, left and right; and, each loudspeaker may be positioned at equal radius from the center where the subject 2 is positioned. It should be understood that the method is not limited to one shape configuration of loudspeakers and/or a fixed amount and positions of loudspeakers, and that embodiments may include any amount of loudspeakers in any spatial configuration thereof.

In an embodiment, the loudspeakers 4 used for such configuration may be omnidirectional, i.e. with equal distribution of the audible frequency range across an angle 90-degrees off-axis to achieve optimal coherence between the configured loudspeakers. It should be understood that the method may include obtaining described effects with any other combination of loudspeakers and/or with any other types of loudspeakers or sound transducers, including but not limited to vibro-transducers, bone-conduction transducers and headphones. It should be understood that the invention may include configuration of devices that project sound within the human audible frequency range as well by devices that project in the ultrasonic range (>20 kHz) and infrasonic range (<20 Hz), which may exceed the generally regarded human audible frequency range.

In an embodiment, the subject 2 is placed in the center of a loudspeaker configuration, thus enabling the subject 2 to receive the acoustic summation of the audio signal equally from all sides. It should be understood that the method may include positioning of the subject 2 in any other position or posture, including laying, sitting, standing and/or moving in space; and, that the subject 2 may experience the described physiological effect of the projected sound shape while being physically positioned inside or outside of the virtual sound source 10.

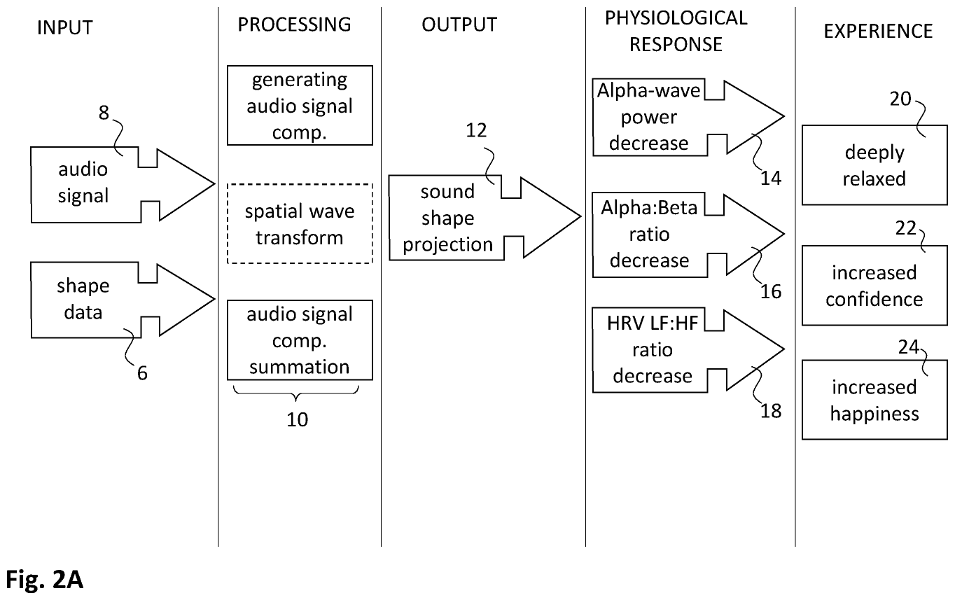

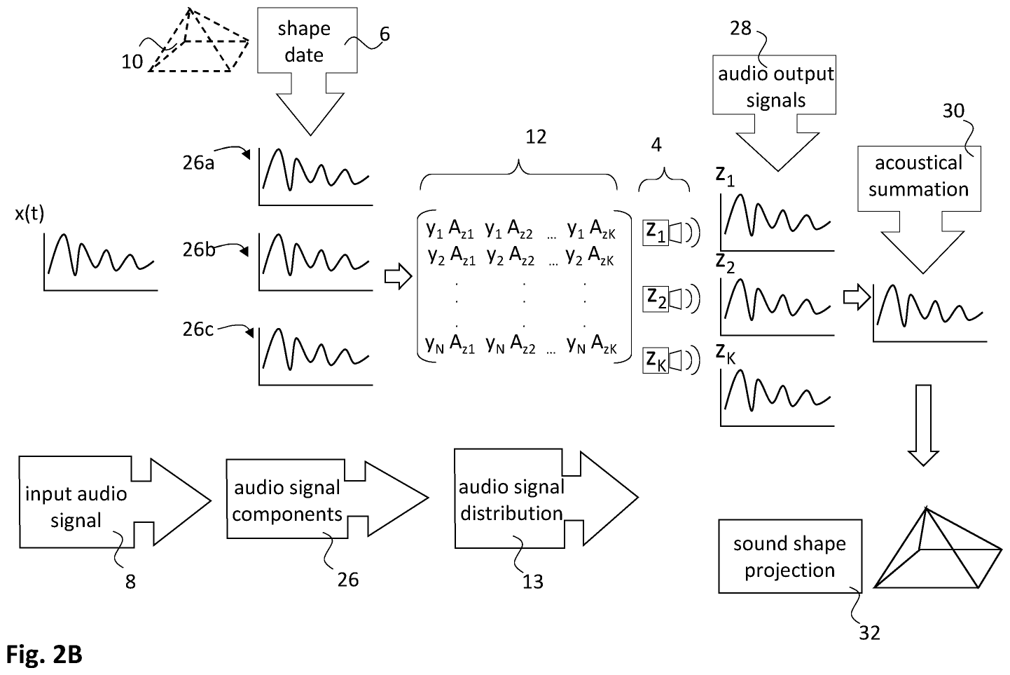

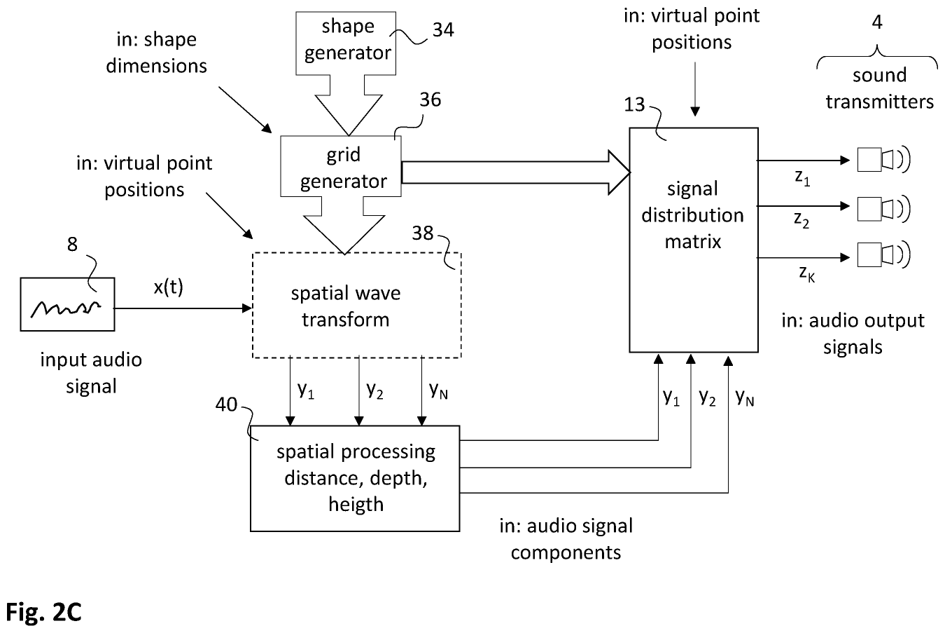

FIG. 2A describes an embodiment with as input one (or more) audio signal(s) 8; and, virtual sound source information 6 defining the virtual points of the virtual sound source and thus defining the spatial dimensions of the virtual sound source 10, such as the shape and/or size and/or density of the virtual sound source 10 and/or the position of the virtual sound source 10 with respect to the subject 2.

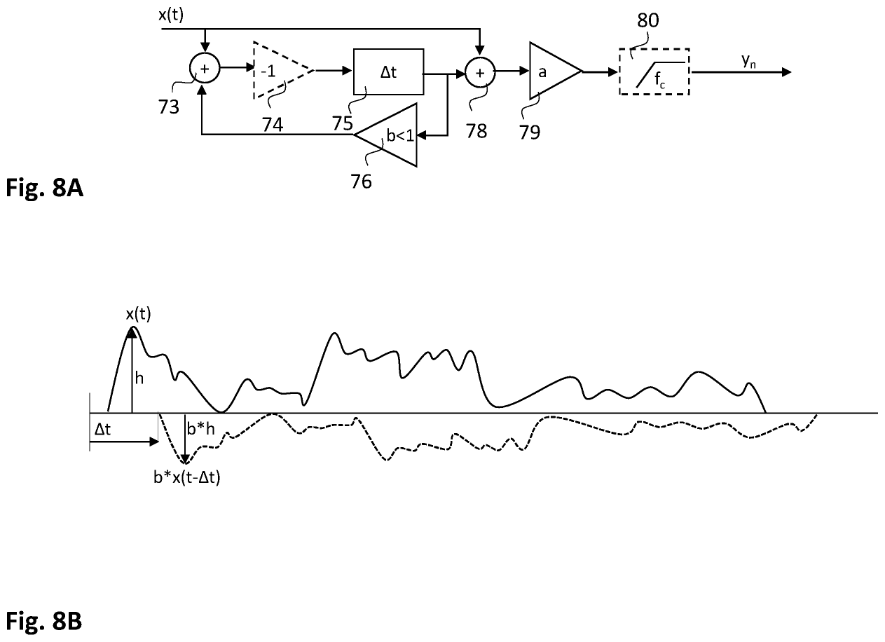

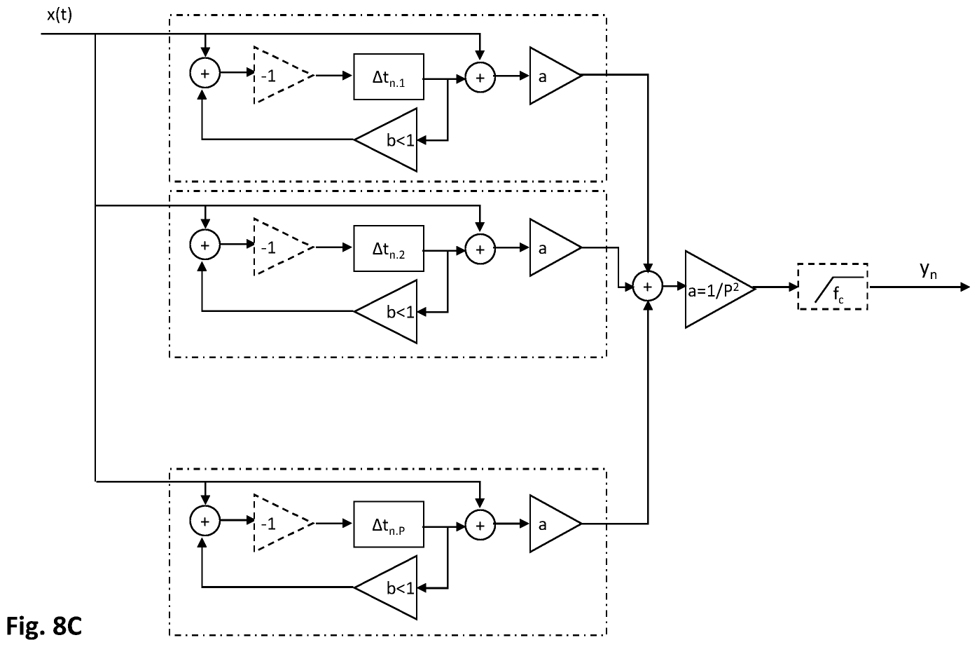

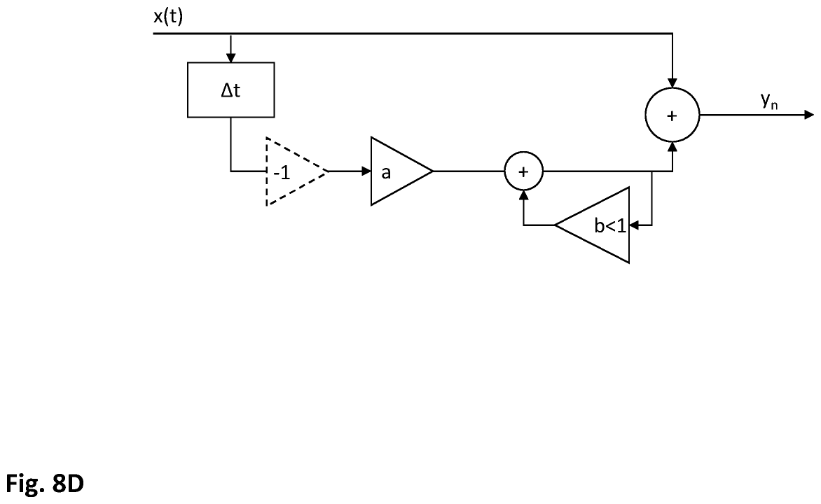

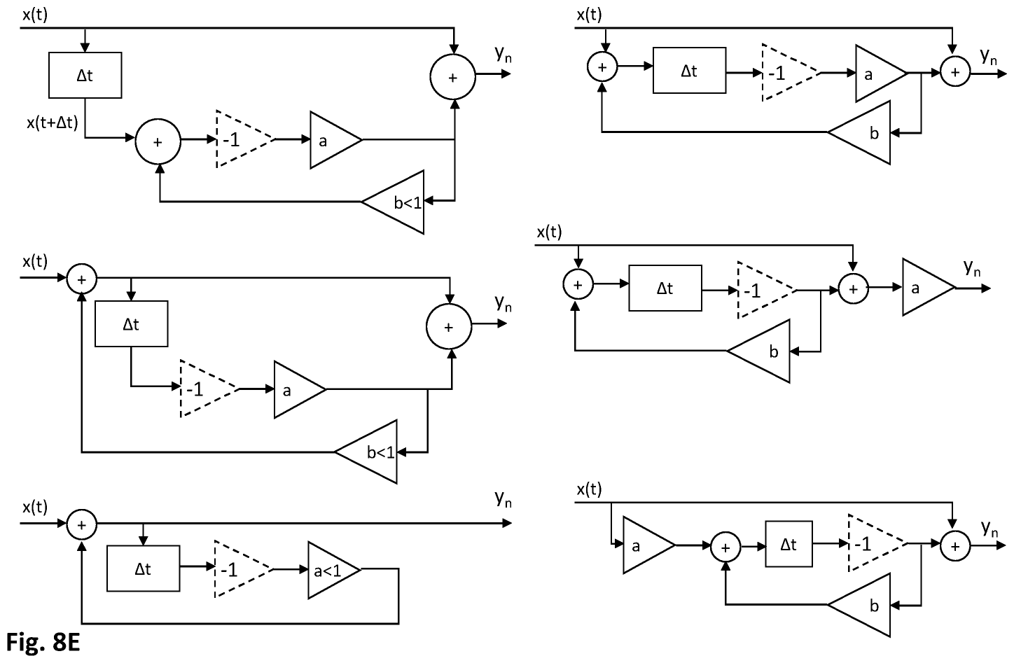

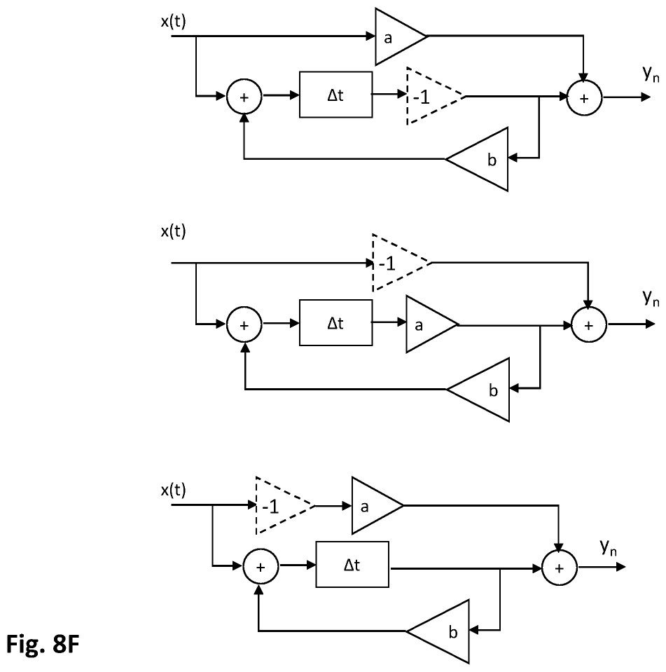

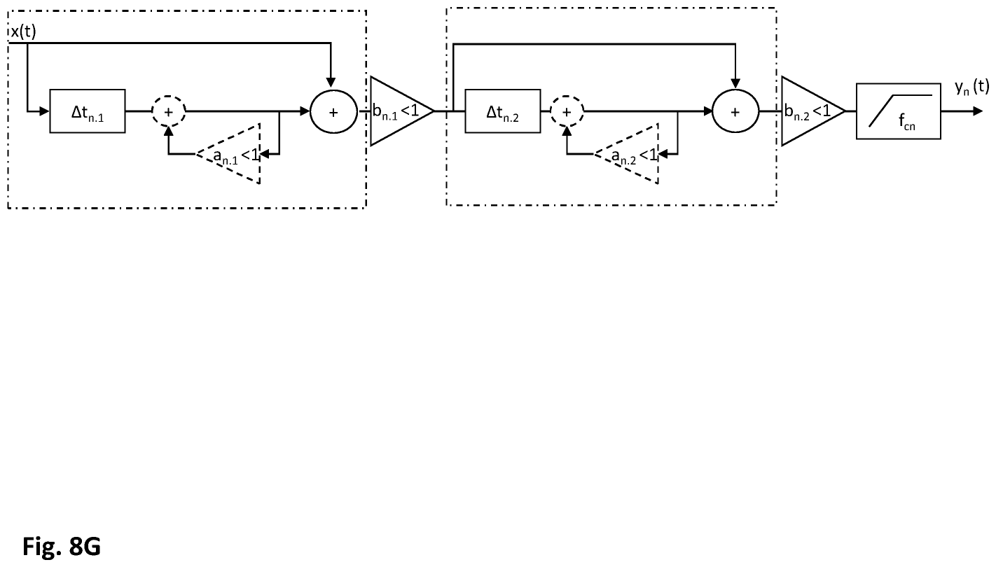

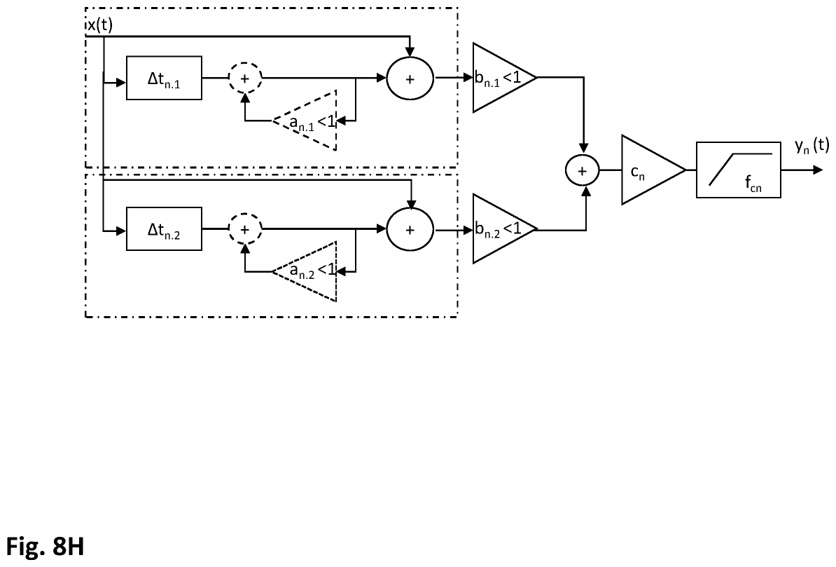

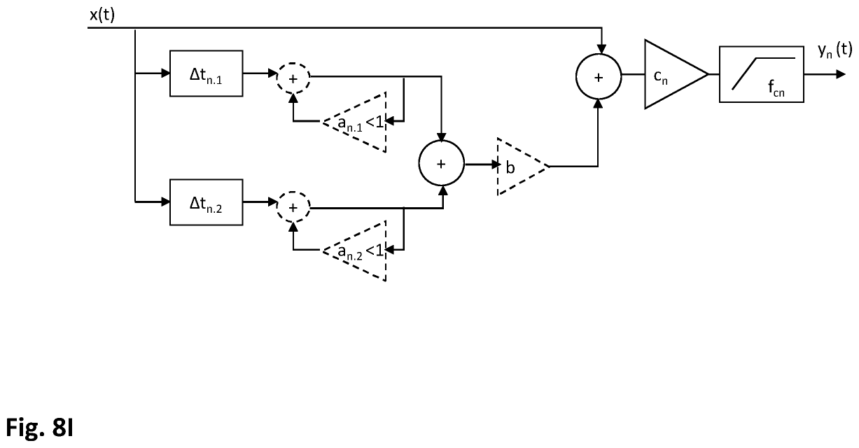

The processing comprises, in an embodiment, associating the input audio signal with a distinct shape, ie. modifying the input audio signal based on the virtual sound source information and generating audio signal components for respective virtual points that define the virtual sound source. Optionally, a spatial wave transform operation is performed when determining each audio signal component. Such spatial wave transform is described with reference to FIG. 8A. The audio signal 12 that is provided to the subject 2 comprises the audio signal components respectively associated with the virtual points. The audio signal 12 may be panned to a plurality of loudspeakers 4.

The audio signal 12 provided to the subject 2, which the subject 2 perceives as originating from a virtual sound source 10 having a shape and position, may be said to form a projection of the virtual sound source 10 with that shape. The virtual sound source 10 besides a shape also has a position relative to the subject 2 and may also have a certain density. The virtual points may define the density of the virtual sound source 10 in that a higher density of virtual points per volume corresponds to a higher density of the virtual sound source 10 The physiological response to the sound shape projection 12, for example indicating improved homeostasis, may be measured by a significant decrease 14 in Alpha-wave mean power and a significant decrease 16 in Alpha:Beta-wave power ratio in the Brain Activity; and, a significant decrease 18 in LF:HF power ratio in the Heart Rate Variability (HRV) where LF stands for “Low Frequency” and HF for “High Frequency”. The described effects, i.e. improvement of the physiological state of the subject 2, may be observable within a short exposure period to the audio signal, e.g. less than 5 minutes.

The experience described by the subject 2, as a result of being provided the audio signal, are associated with feelings in the subject 2 of deep relaxation 20, i.e. significantly more relaxed and less nervous after exposure than before exposure; increased confidence 22, i.e. more confidence and less anxiety after exposure than before exposure; and, increased happiness 24, i.e. more happy and less frustrated and/or less depressed after exposure than before exposure.

FIG. 2B describes an embodiment wherein the virtual sound source 10 is defined by a plurality of virtual points. Each virtual point has a virtual position with respect to other virtual points, and with respect to the subject 2. Further, the audio signal 12 comprises a plurality of audio signal components, wherein each audio signal component of the plurality of audio signal components is respectively associated with a virtual point of the plurality of virtual points.

FIG. 2B illustrates how the audio signal can be obtained. The virtual sound source information 6 defines the respective positions of the virtual points. The input audio signal 8 is taken as input. Then, the respective audio signal components 26 for the plurality of virtual points are determined based on the input audio signal 8 and based on the respective positions of the plurality of virtual points. For each audio signal component 26_x, determining the audio signal component 26_x comprises modifying the input audio signal 8 to obtain a modified audio signal component using a signal delay operation introducing a time delay; and determining the audio signal component based on a combination, e.g. a summation, of the input audio signal, or of an inverted and/or attenuated or amplified version of the input audio signal, and the modified audio signal component.

The audio signal 12 may then be distributed to several loudspeakers 4 using a signal distribution matrix 13 as will be explained in more detail below.

The acoustical summation 30 of the audio output signals 28 thus obtained for each discrete loudspeaker zₙ in a loudspeaker configuration results in a sound shape projection 32, i.e. a sound source has a distinct shape, size and is positioned at a particular distance, height and depth in relation to the subject 2. The generated audio signal 12, once played out by a loudspeaker system 4, can be considered a projection of the virtual sound source's shape irrespective of how many loudspeakers are used and irrespective of the position of the observer 2 relative to the loudspeakers 4. The described sound shape projection (at least partially) overrules the spatio-spectral properties of the individual loudspeaker(s) and creates a coherent spatial projection of the sound signal by means of its size and shape. This is also described in patent applications NL2024434 and NL 2025950 describing a method to associate an audio signal with a virtual sound source, the contents of which should be considered included in this disclosure in its entirety.

FIG. 2C describes an embodiment with as input an audio signal denoted “x(t)”. In this embodiment, a ‘shape generator’ 34 generates data representing a dimensional shape. A ‘grid generator’ 36 takes as input this data representing a dimensional shape and generates a grid of equally distributed virtual points on the dimensional shape. Such grid may be referred to as virtual sound source information as it defines the positions of the virtual points constituting the virtual sound source. The virtual sound source information at least defines the virtual points' virtual positions with respect to each other and their positions with respect to the subject 2.

The virtual sound source information 6 can then subsequently be used to modify the input audio signal 8 by, optionally, applying a ‘spatial wave transform’38 relative to the dimensional shape of the virtual sound source, e.g. determine a plurality of audio signal components 26_x respectively associated with the virtual points as defined by the virtual sound source information. The respective positions of the virtual points may be denoted in Cartesian coordinates (x, y, z).

The audio signal components 26 are further modified based on the distance, height and depth relative to the subject 2 of their associated virtual points. The resulting audio signal components 26 may then be input to a ‘signal distribution matrix’ 13 with as input the optionally modified audio signal components y(t)ₙ and particle positions, i.e the virtual position of each determined point on the virtual shape generated by the particle grid generator, optionally denoted in Cartesian coordinates (x, y, z).

The signal distribution matrix 13 can then distribute the audio signal to a plurality of loudspeakers 4 as described in more detail below.

Once the audio signal 8 is provided using the loudspeakers 4 to the subject 2, the subject 2 will perceive the audio signal 8 as if it originates from a virtual sound source 10 having the shape as output by the shape generator 34.

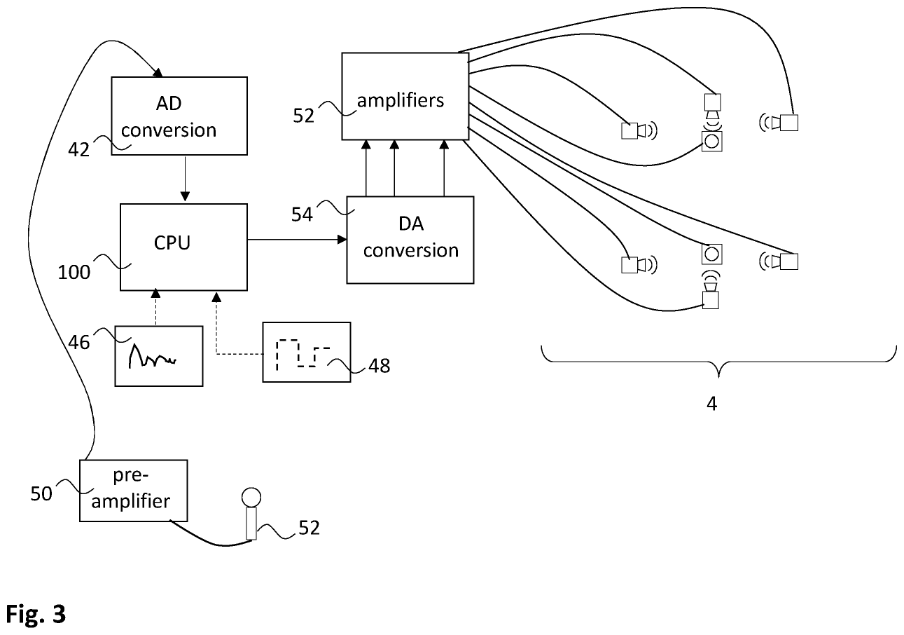

FIG. 3 describes a system and/or method for providing the audio signal 12 to a subject 2 for improving the physiological condition of the subject 2. In an embodiment, the system comprises a microphone actuator 52 or any other of type pressure-velocity transducer for generating an input audio signal 8 based on sound waves hitting such pressure velocity transducer. The system may comprise a pre-amplifier 50 that is configured to amplify the input audio signal 8 as generated by the pressure-velocity transducer 52. The system further comprises an analogue to digital converter 42 in order to convert the analogue input audio signal into a digital version. The system further comprises a data processing system 100 configured to process the audio signal based on the virtual sound source information in manners described herein. The system also comprises a digital to analogue converter 54 that is configured to convert the digital audio signal as output by the data processing system to an analogue version. The system further comprises one or more amplifiers 52 for amplifying the resulting audio signal before feeding it to a plurality of loudspeakers 4, which the system also comprises. The system may comprise an amplifier for each loudspeaker. Further each loudspeaker may be connected its own amplifier by means of its own audio cable.

In light of this system, it is clear that the method for improving the physiological condition of a subject may comprise generating an input audio signal based on sound waves hitting such pressure velocity transducer, amplifying the input audio signal as generated by the pressure-velocity transducer, and converting the analogue input audio signal into a digital version, and processing the audio signal based on the virtual sound source information in manners described herein, and convert the analogue audio signal as output by the data processing system to an analogue version, and amplifying the resulting audio signal before feeding it a plurality of loudspeakers. Herein, amplifying the resulting audio signal may comprise separately amplifying each loudspeaker audio signal.

The system and method as depicted in FIG. 3 allow to acquire the input audio signal using a pressure-velocity transducer 52, e.g. a microphone, determine the audio signal and provide it to the subject 2, all in real-time.

In another embodiment, the audio input signal(s) 46 may have been output by a recording process in which sounds have been acquired or generated prior to playback and stored onto a readable digital or analogue storage medium;

In another embodiment, the audio input signal(s) 48 have been output by means of a digital or analogue synthesis process, acquired prior to playback and stored onto a digital or analogue storage medium; and/or acquired in real-time and/or optionally converted into a digital signal.

This disclosure also relates to a computer processing unit 100, also referred to as a data processing system, that executes computer program and/or code portion designed to modify an audio input signal and generate modified audio signal components associated with points on a virtual shape; and, generate audio signal components associated with a discrete loudspeaker as part of a loudspeaker configuration, i.e. audio output signals.

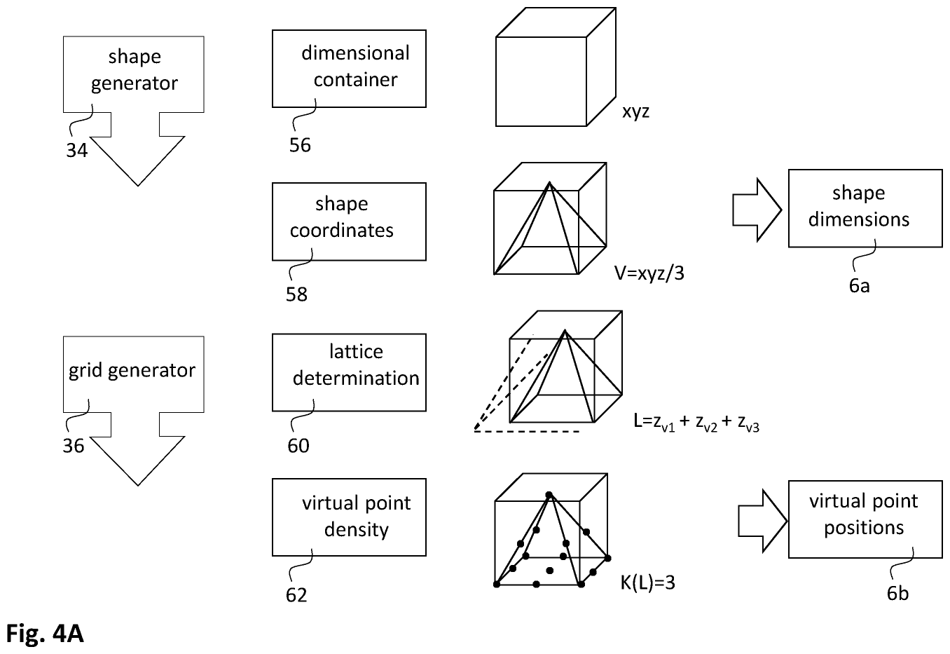

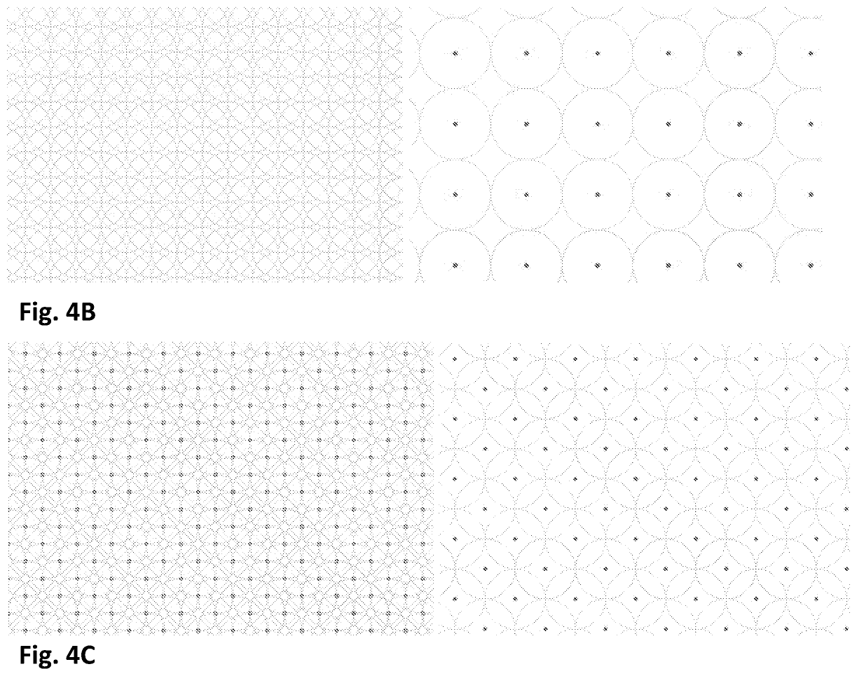

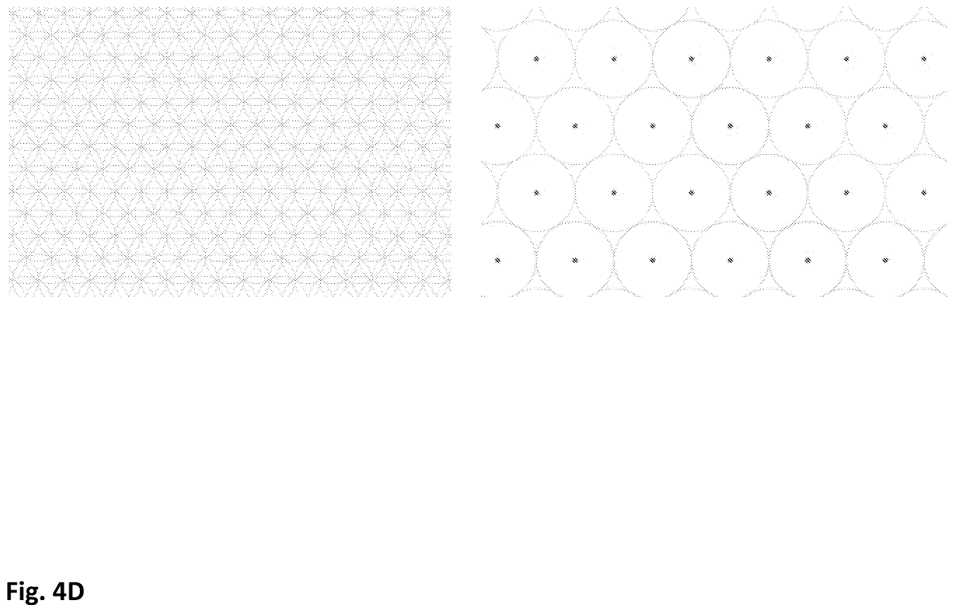

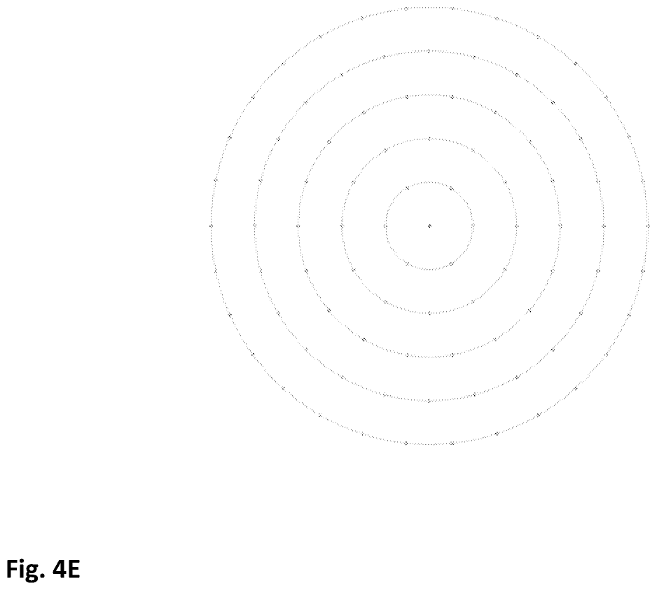

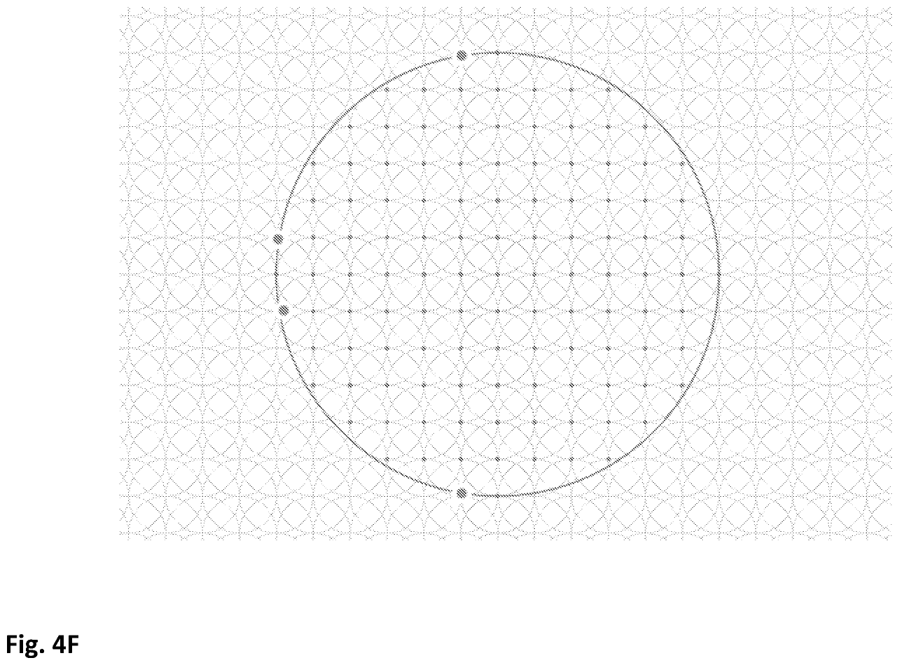

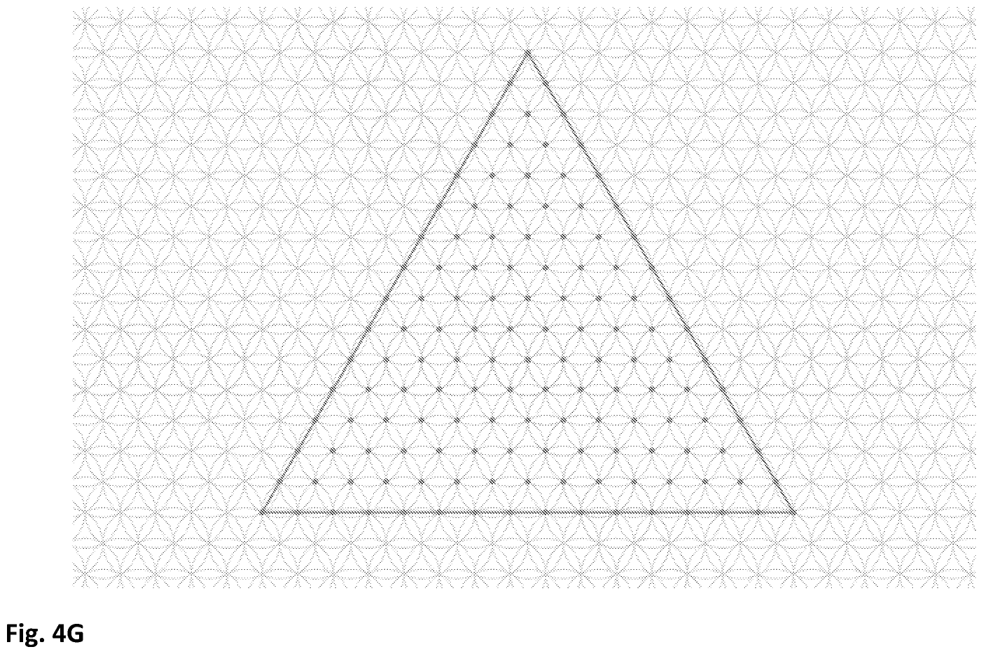

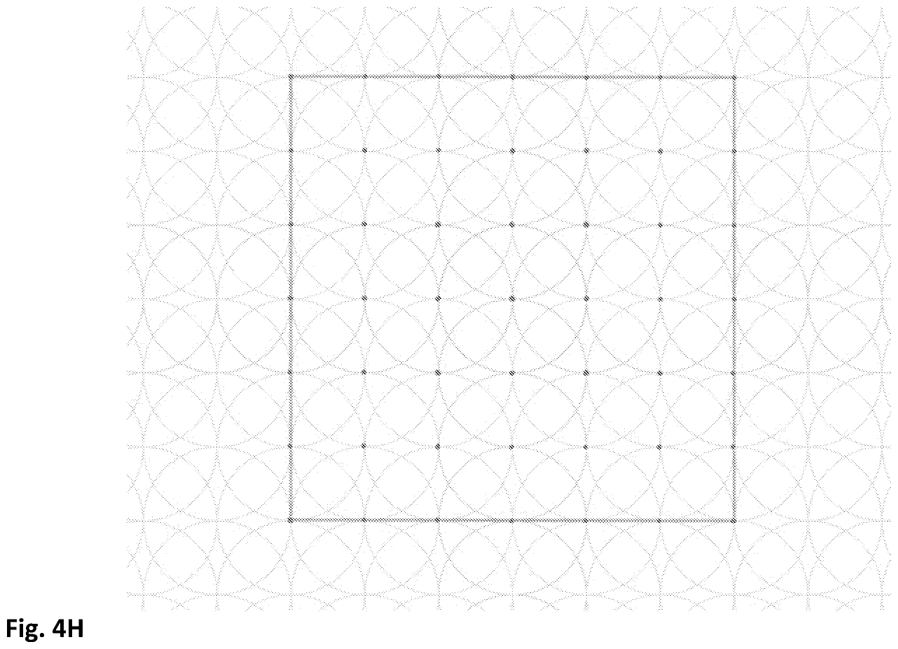

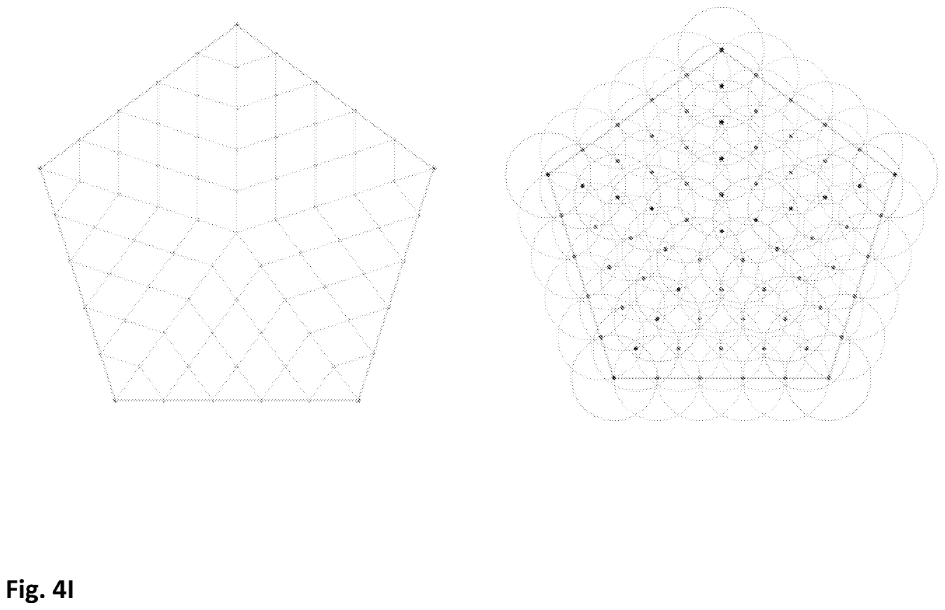

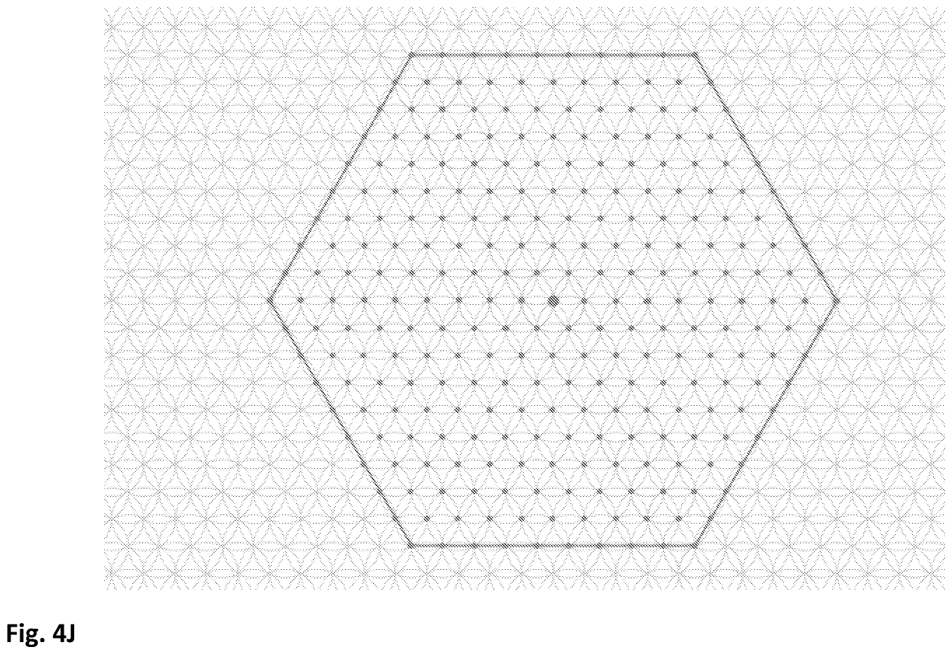

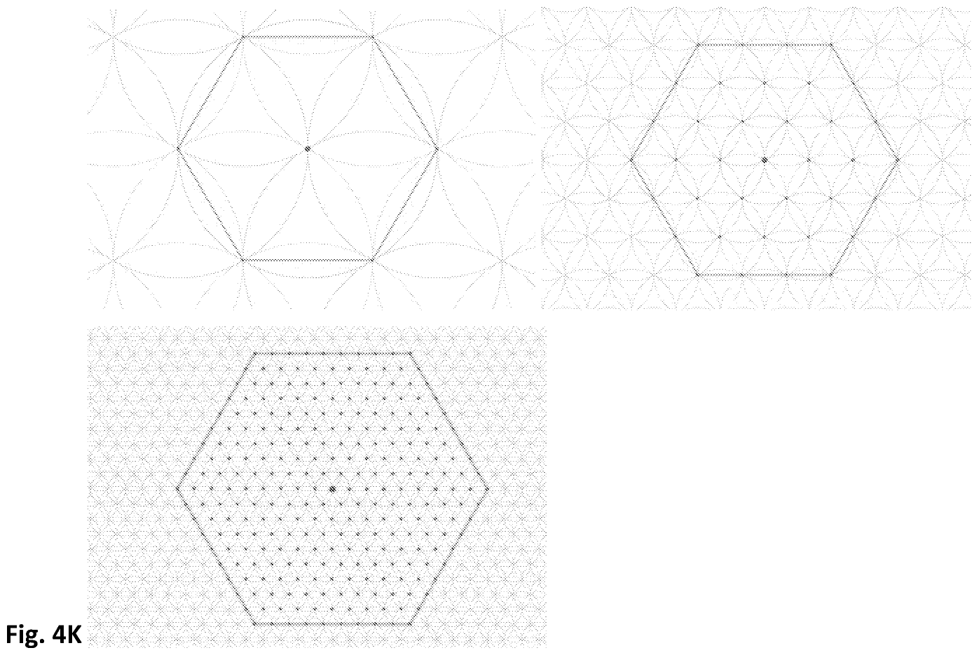

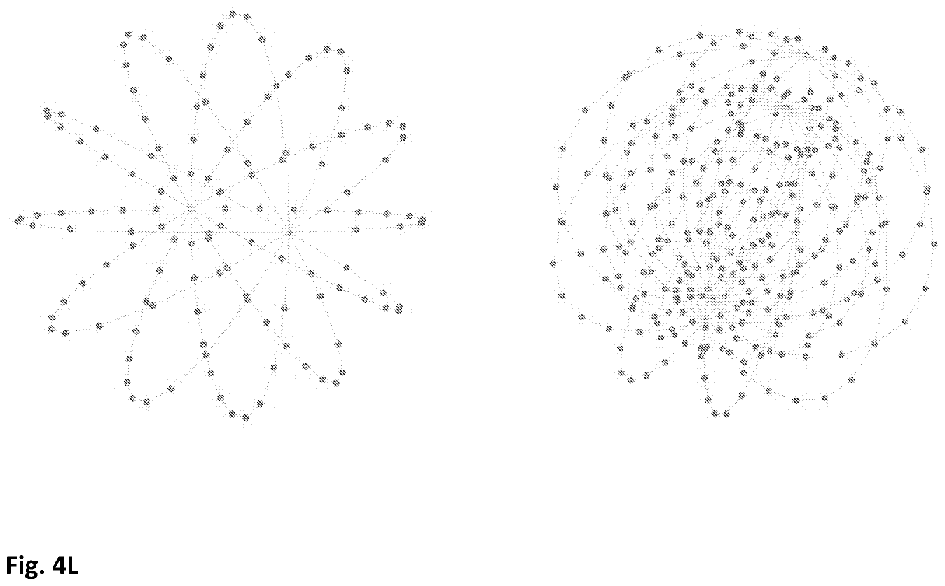

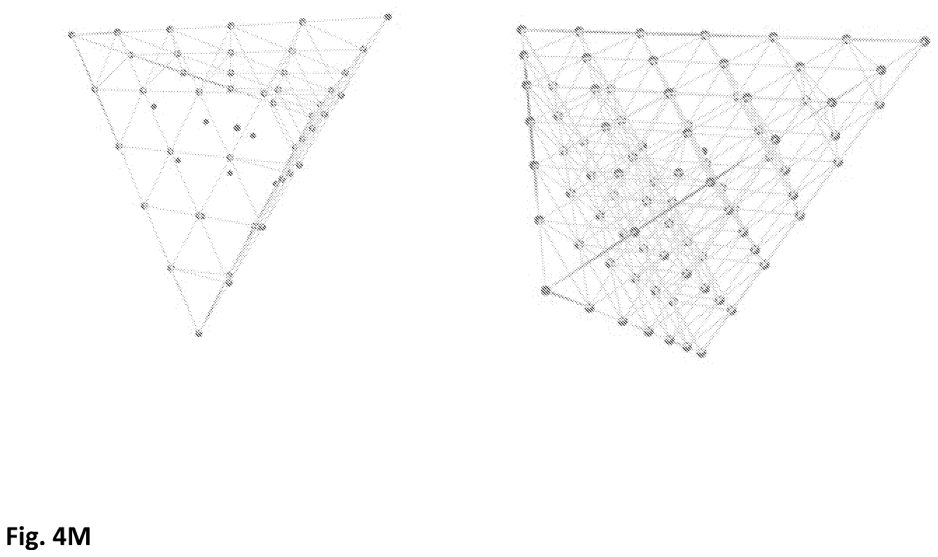

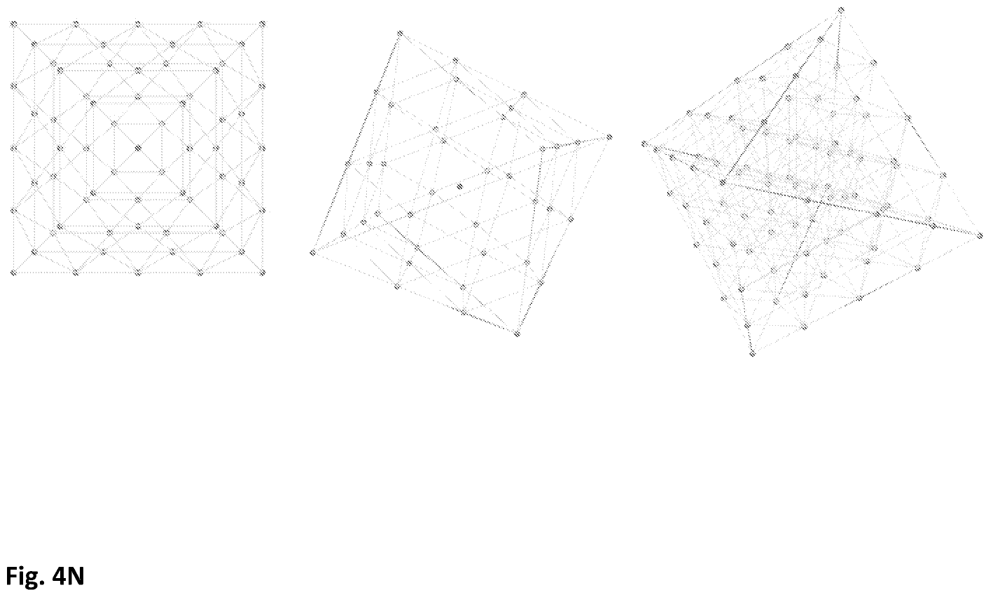

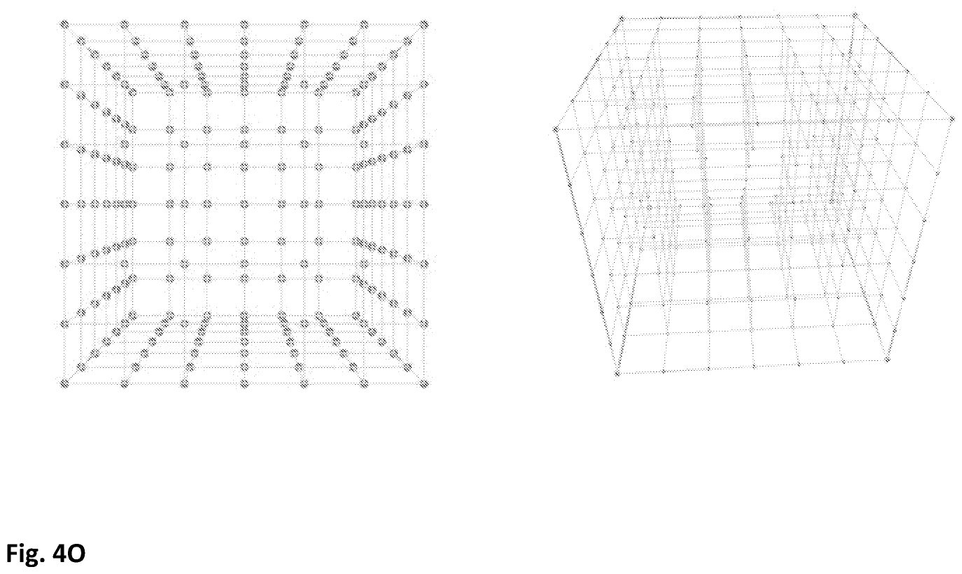

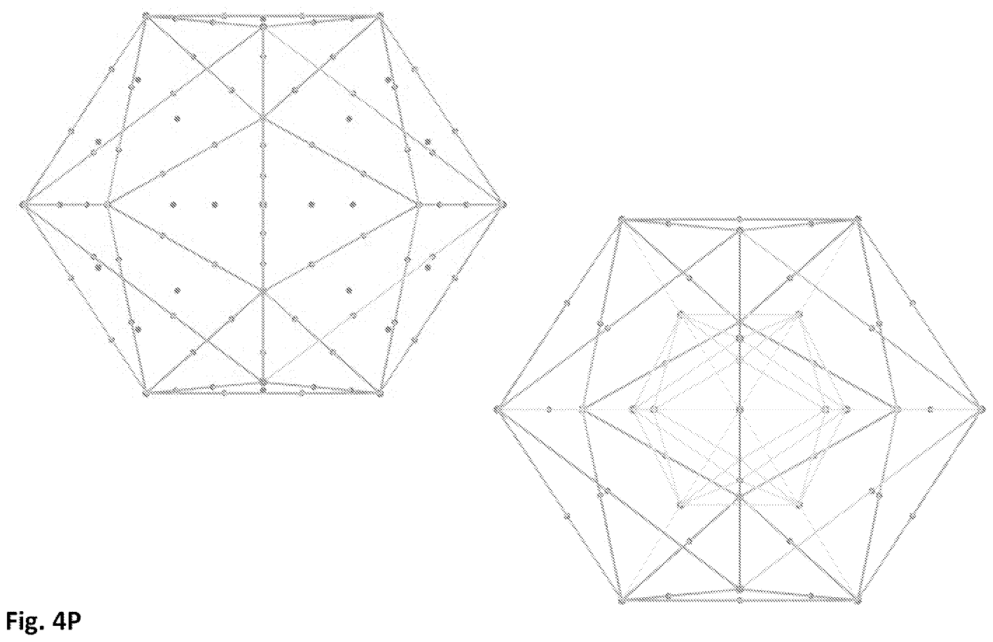

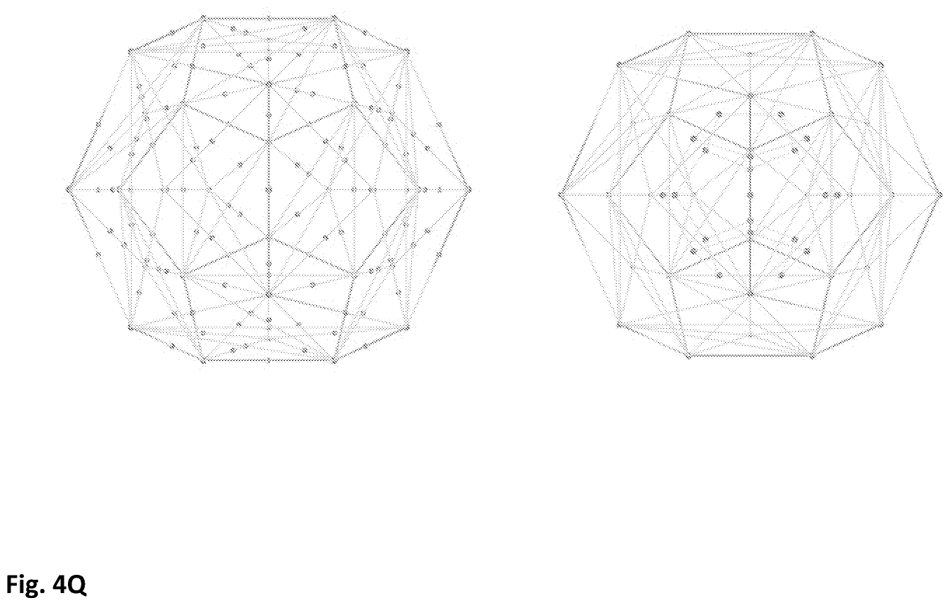

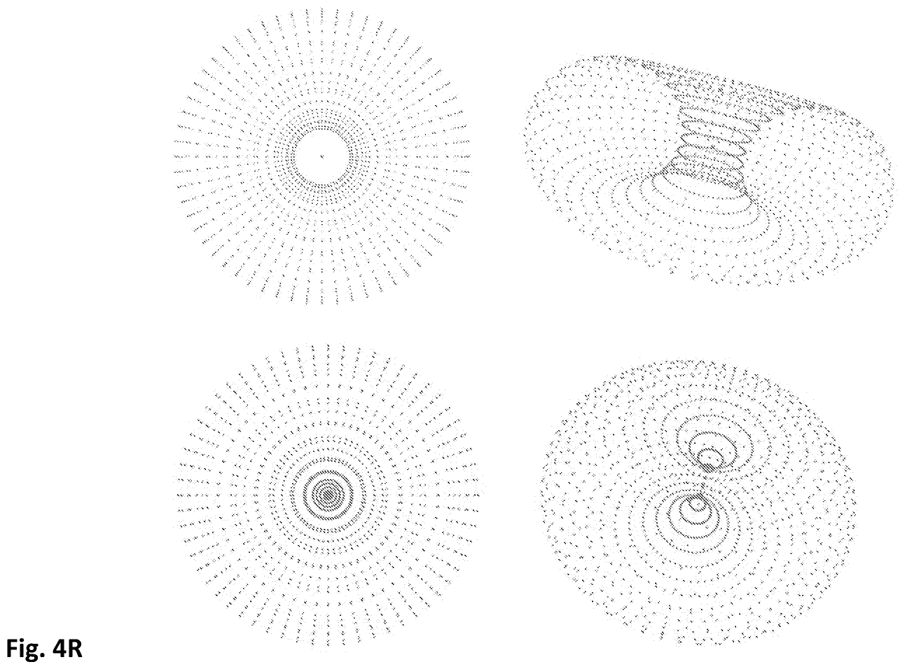

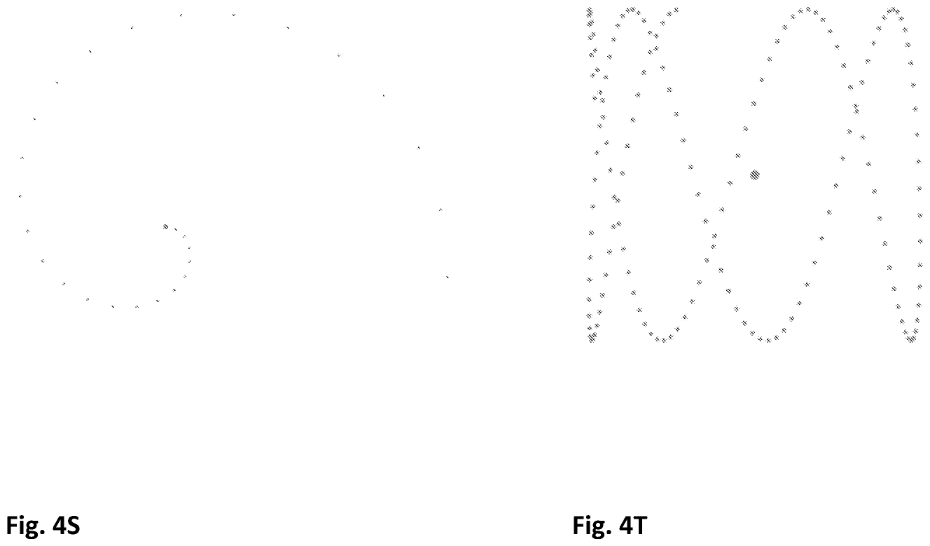

FIG. 4A illustrates a method to determine the virtual sound source information 6 as described herein. The virtual sound source information 6 indicates the spatial dimensions of a virtual object 10, i.e. the shape and size and its position relative to the subject 2, and, optionally, the density of a virtual sound source 10.

The virtual points may be equally distributed over the surface of the virtual sound source 10. A higher density of the virtual points on such surface corresponds to a higher resolution.

It should be appreciated that the virtual sound source 10 can be defined to be hollow. In such case, the virtual sound source information 6 does not define virtual points “inside” the virtual sound source 10, but only on the external surfaces and edges of the virtual sound source 10. The virtual sound source 10 can also be “solid”. In such case, the virtual sound source information 6 defines, in addition to virtual points on the exterior surfaces and edges of the virtual sound source 10, virtual points “inside” the virtual sound source 10, which may be equally distributed across the interior volume of the virtual sound source 10.

In an embodiment, a virtual sound source 10 has a geometric shape, i.e. a pure dimensional shape, or semi-geometric, irregular or may be organically shaped. It should be understood that the virtual sound source 10 may have any form and that any method may be used to determine the shape of the virtual sound source and the virtual points constituting that shape.

The density of the virtual points may also be referred to as the resolution of the virtual points and/or the ‘grid resolution’.

FIG. 4A illustrates that obtaining the virtual sound source information may comprise dimensions of the virtual sound source 6a and the virtual point positions 6b. Obtaining the shape dimensions 6a may comprise a shape generator 34 generating a container 56 of scalable dimensions (xyz) and determining shape coordinates 58 and a shape volume within the boundaries of the scaled dimensions to obtain the dimensions of the virtual sound source 10. In the depicted example, the virtual sound source 10 is shaped as a pyramid. Furthermore, obtaining the virtual point positions 6b, may comprise a grid generator 36 determining a lattice 60, where three main lattices are introduced in accordance with the dimensions of chosen shape; and, determining the virtual point density 62 by defining a resolution of points along each of the introduced lattices, to obtain the virtual point positions within a shape.

An infinite lattice L can be defined as

L=a.(Z.v_1+Z.v_2+Z.v_3)
where Z is the ring of integers, and v_1, v_2, v_3 describe three vectors and constant a relates to the minimal increment as
={points (x,y), such that x=a.n.(v_1.x)+a.m.(v_2.x), y=a.n.(v_1.y)+a.m.(v_2.y), with n, m integers.}

As it is considered that sound propagates symmetrically in all directions, the patterns of overlapping or tangent circles generated by the lattice is considered, where a sphere is centered around each virtual point of the grid. The radius of the circles may be increased to influence the generated patterns of the sound propagation in space, which are further described in the following examples.

FIG. 4B shows an orthogonal lattice_2 with the vectors v_1(1,0), v_2(0,1); with on the left the overlapping circles of radius a, the centers of the circles being the points of the grid; and, on the right right, with tangent circles of radius a/2.

FIG. 4C shows a center square lattice_4 with the vectors v_1(1,0), v_2(1/2,1/2); with, on the left, overlapping circles of radius a, the centers of the circles being the points of the grid; and, on the right, with overlapping circles of radius a/2.

FIG. 4D shows a triangular lattice_3 with the vectors v_1(1,0), v_2(1/2, √3/2); with on the left, overlapping circles of radius a; and, on the right, with tangent circles of radius a/2.

FIG. 4E shows an embodiment of the invention where a shape is a circle on a lattice with a finite grid. The k circle consists of 6*k points with a hexagonal symmetry of rotation 2π/6 equivalent of a ‘nested circle grid’. Each k-circle (k from 0 to res) has radius

k*R/res

where R is the radius of the actual shape and has 6*k points on it. The 0-circle is the center point, while res-circle is the actual shape.

FIG. 4F shows an embodiment where a shape is a circle and based on Lattice_2. In an embodiment, only those points of the lattice that are inside the shape are included in the grid. In another embodiment, additional points may be added to the grid which have enough vicinity to the boundary of the shape to be taken into account, a ‘boundary correction index’.

FIG. 4G shows an embodiment of the invention where a shape is a triangle based on Lattice_3 equivalent to a ‘nested triangle grid’. Each k-triangle (k from 0 to res) has length

k*L/res

where L is the side length of the actual shape and has 9*k points, or 3*k for each edge.

FIG. 4H shows an embodiment of the invention where a shape is a square based on Lattice_2, or Lattice_4 equivalent to a ‘nested square grid’. Each k-square (k from 0 to res) has length

k*L/res

where L is the side length of the actual shape and has 8*k points, or 2*k per edge.

FIG. 4I shows an embodiment of the invention where a shape is a pentagon based on a grid with regular tessellation, equivalent to a ‘nested pentagon grid’. Here the tessellation is regular but the points are not fully equidistant but icosele. Each k-pentagon (k from 0 to res) has radius

k*R/res

where R is the radius of the actual shape and has 5*k points, or k per edge.

FIGS. 4J and 4K show an embodiment of the invention where a shape is a hexagon based on Lattice_3, equivalent to a ‘nested hexagon’ grid. Each k-hexagon (k from 0 to res) has radius

k*R/res

where R is the radius of the actual shape and has 6*k points, or k per edge.

To determine the number of points within a shape, i.e. the grid resolution, res=resolution and integer >=0. If res=0 then only one point is positioned in the center. If res=1, one point is positioned at the center and one on each vertex; etc. FIG. 4K describes res=1, res=3 and res=9 for the hexagon shape, respectively.

FIG. 4L shows an embodiment of the invention where a shape is a nested sphere, with on the left a hollow sphere, and on the right a solid sphere. For a hollow shape, a grid of points is applied on the faces of the shape only, similar to only positioning points on the edges of a 2-dimensional shape according to the chosen resolution. From each determined grid of a solid shape, the resolution of the hollow shape can be deduced. The hollow shape corresponds to omitting all points that are not located on the boundaries of the nested full shape.

The k sphere (0<k<res) has radius k*a and in an embodiment the sphere is composed of 3*k circles joining at height with 6*k points on each circle.

FIG. 4M shows an embodiment of the invention where a shape is a nested tetrahedron, with on the left a hollow tetrahedron, and on the right a solid tetrahedron. Each k-tetrahedron (k from 0 to res) has radius X, where R is the radius of the actual shape and has X points, or k per edge.

FIG. 4N shows an embodiment of the invention where a shape is a nested octahedron, with on the left a hollow octahedron, and on the right a solid octahedron. Each k-octahedron (k from 0 to res) has radius X, where R is the radius of the actual shape and has X points, or k per edge.

FIG. 4O shows an embodiment of the invention where a shape is a nested cube, with on the left a hollow cube, and on the right a solid cube. Each k-cube (k from 0 to res) has radius X, where R is the radius of the actual shape and has X points, or k per edge.

FIG. 4P shows an embodiment of the invention where a shape is a nested icosahedron, with on the left a hollow icosahedron, and on the right a solid icosahedron with res=2. Each k nested icosahedron is decomposed into 20 triangular faces each of which is decomposed in a grid, according to the resolution=k, as the triangular decomposition, with the exception of imposing a center point in the triangle.

FIG. 4Q shows an embodiment of the invention where a shape is a nested dodecahedron, with on the left a hollow dodecahedron, and on the right a solid dodecahedron with res=2. Each k nested dodecahedron is decomposed into 12 pentagonal faces, each of which is then decomposed in a grid, according to the resolution=k as given by the chosen mesh for the pentagon.

In an embodiment, a shape can be a swarm, a cluster of bounded points that bounce within the area or boundaries of a dimensional shape, or forming an infinite, deterministic or probabilistic transformation of shape.

FIG. 4R shows an embodiment of the invention where a shape is a nested torus, where the number of nested torus per default=res, but may be added as a parameter. The virtual point positions within a dynamic grid are given by

x=r(a+cos v)cos u y=r(a+cos v)sin u z=r sin v

A torus shape can then transform into 3 types by modifying the parameters for r and a. If a=1 a ‘horn torus’ if formed; if a<1 a ‘spindle torus’ is formed; if a>1 a ‘ring torus’ is formed.

FIG. 4S shows an embodiment of the invention where a shape is an Archimedean spiral, where the virtual point positions within a dynamic grid are given by

r=a.u+b: x=(a.u+b)*cos(u)y=(a.u+b)*sin(u)z=0

FIG. 4T shows an embodiment of the invention where a shape is a helix, where the virtual point positions within a dynamic grid are given by

x=r.cos(a.u)y=r.sin(b.u)z=u with r,a,b fixed

or the helicoid variant

x=r.cos(a.u)y=r.sin(a.u)z=u

where, for instance: 1≤r≤1 and −π≤u≤π, or else in −inf, +inf.

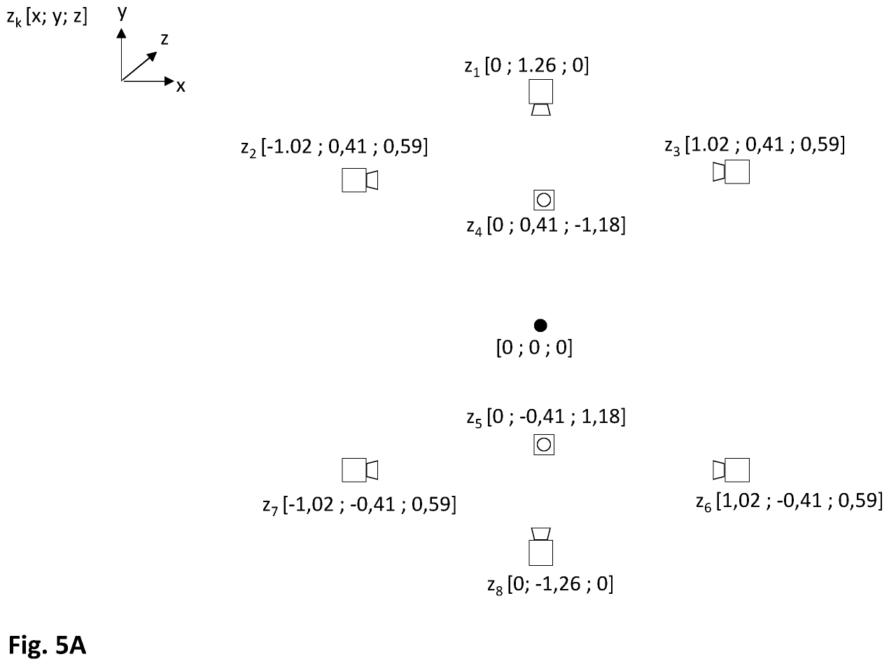

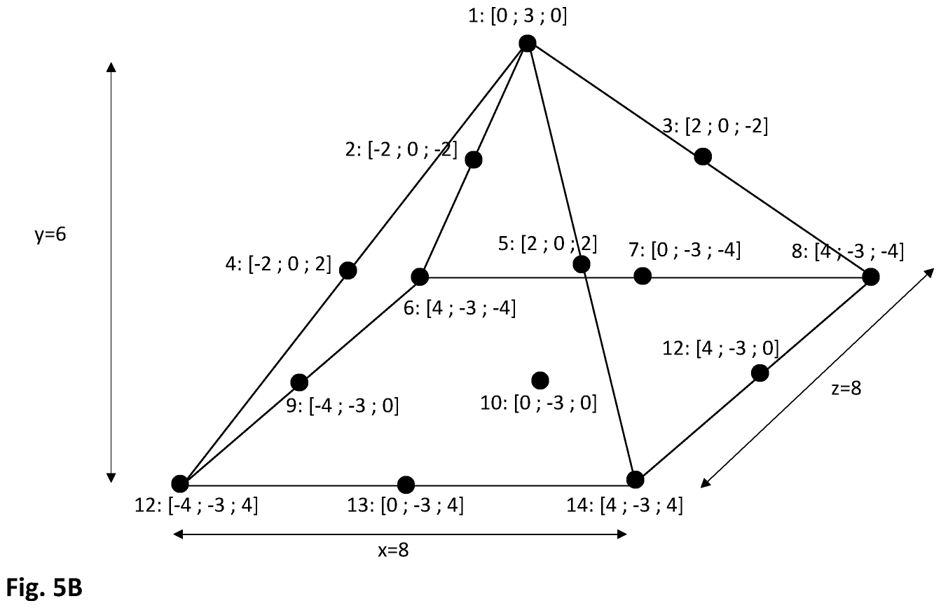

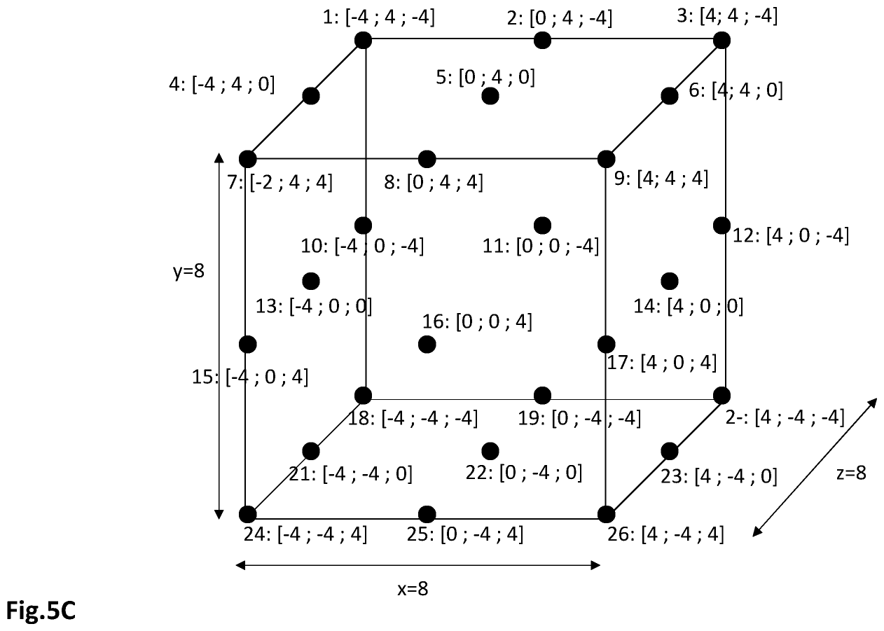

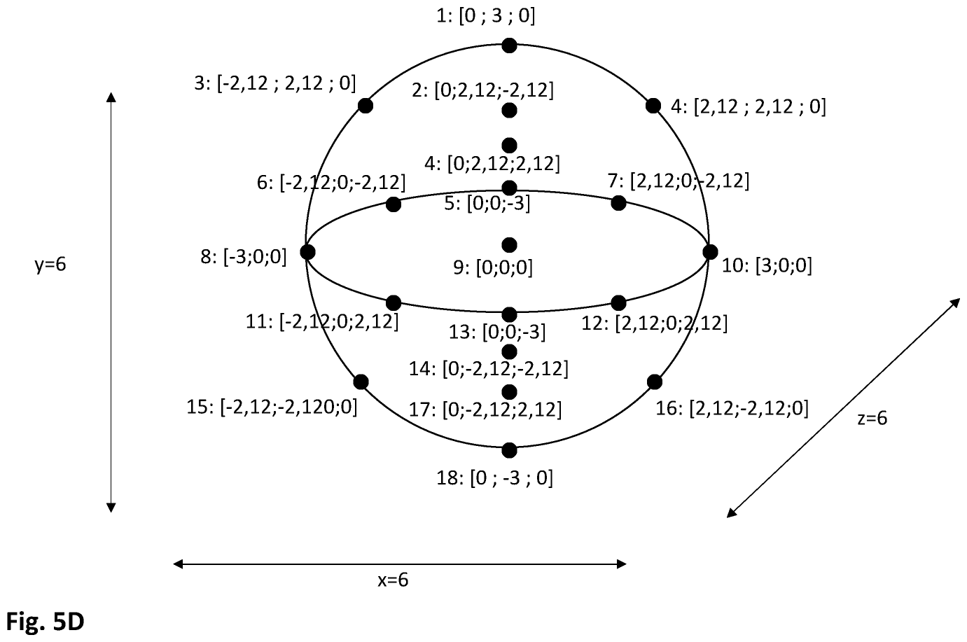

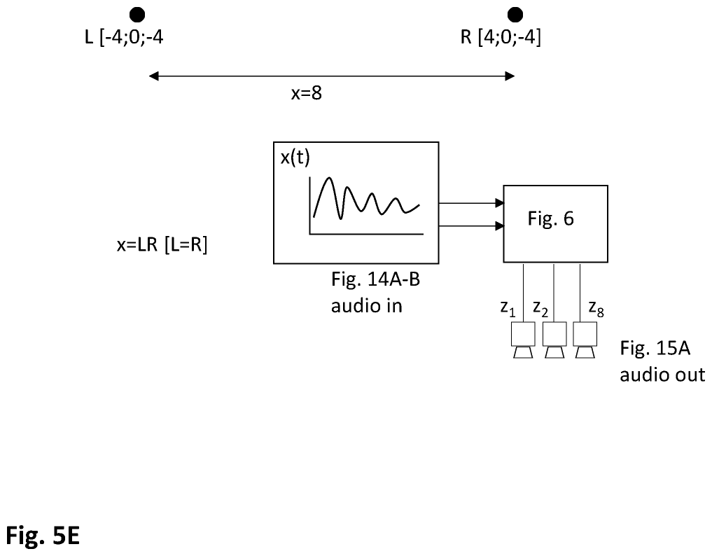

FIG. 5A shows a loudspeaker configuration according to an embodiment that consists of 8 loudspeakers z_1-z_8 positioned with equal radius from a center [0,0,0] and equally above, below, front, back, left and right from a center, forming a ‘tilted cube’, or ‘star-tetrahedron’ shape, i.e. the loudspeaker configuration shape in an embodiment of the invention. This loudspeaker configuration was used for tasks 2-4 described below.

FIG. 5B illustrates a virtual sound source 10 shaped as a pyramid with dimensions 8 meters along the x-direction, 6 meters along the y-direction and 8 meters along the z-direction, also denoted herein as x.8 y.6 z.8 and a grid with lattice k=3—used for task 2 described below.

FIG. 5C illustrates a virtual sound source 10 shaped as a hollow cube with dimensions x.8 y.8 z.8m and a grid with lattice k=3—used for task 3 described below.

FIG. 5D illustrates a virtual sound source 10 shaped as a solid sphere with Dm=6 and a grid with lattice k=1.33—used for task 4 described below.

FIG. 5E is a loudspeaker configuration according to an embodiment that is a stereo setup. [L=R] used for task 1 described below.

FIG. 5F illustrates the signal processing that was employed for tasks 2-4 described below. Note that the signal processing for tasks 2-4 does not involve performing a spatial wave transform as referred to in FIGS. 8A-C.

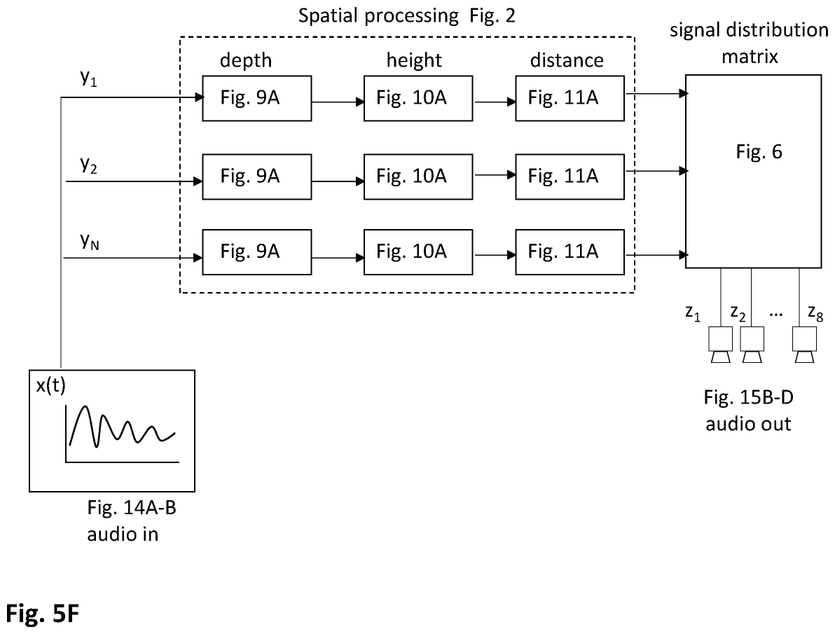

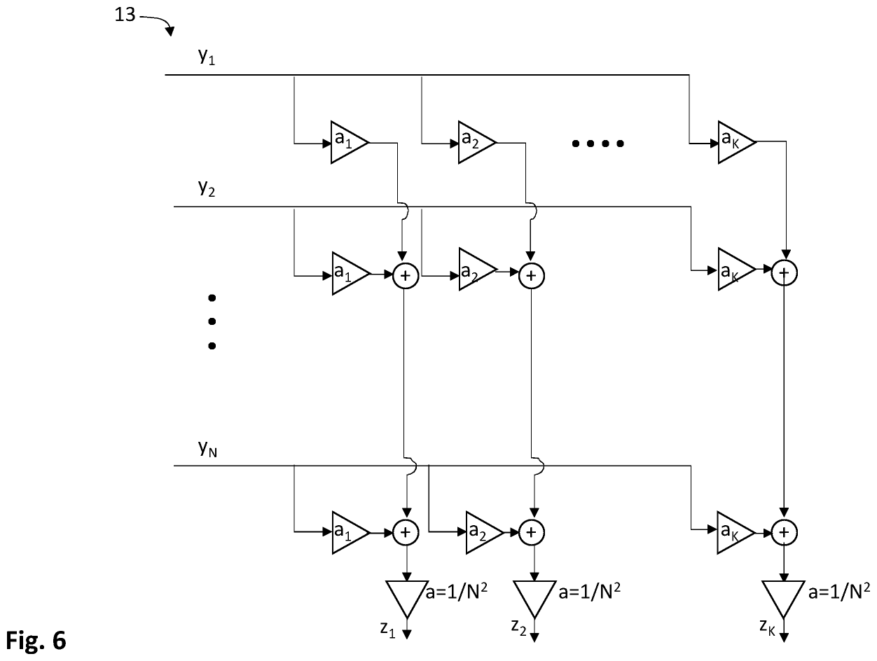

FIG. 6 is a flow chart illustrating a method and system for determining a loudspeaker audio signal for each loudspeaker of a plurality of loudspeakers. The depicted method and system may also be referred to as a signal distribution matrix 13. In this embodiment, a loudspeaker audio signal z_k is determined for each loudspeaker k (not shown) of a plurality of loudspeakers. Input to the signal distribution matrix is the plurality of audio signal components associated with respective virtual points of the virtual sound source which plurality of audio signal components y_n have been determined in accordance with methods described herein.

Each loudspeaker k is associated with a loudspeaker coefficient a_k. In the depicted embodiment, determining loudspeaker audio signal z_k for loudspeaker k comprises attenuating each audio signal component y_n based on loudspeaker coefficient a_k in order to obtain a loudspeaker specific set of attenuated audio signal components. A loudspeaker coefficient for a loudspeaker may be determined based on a distance between the loudspeaker in question and the virtual point. Attenuating each audio signal component y_n based on loudspeaker coefficient a_k may involve simply a multiplication y_n*a_k. In such case, the loudspeaker specific set of attenuated audio signal components for loudspeaker k may be described by: {y_1*a_k; y_2*a_k; y_3*a_k; . . . ; y_N*a_k}, wherein N denotes the total number of virtual points defined for the virtual sound source. Subsequently, the audio signal components in this set are combined, e.g. summed, in order to arrive at the loudspeaker audio signal z_k for loudspeaker k. This method is performed for all loudspeakers k.

In this disclosure, values in the triangles, i.e. in the attenuation or amplification operations, may be understood to indicate a constant with which a signal is multiplied. These constants are often indicated by “a” or “b”. Thus, if such value is larger than 1, then a signal amplification is performed. If such value is smaller than 1, then a signal attenuation is performed.

The signal distribution matrix 13 may have a multiplier and a summation at each position where an input line to which an output signal of a multiplier is supplied, crosses an output, as shown in FIG. 6. The multiplier attenuates the signal received from the input line by a prescribed loudspeaker coefficient specified by a controller, such as the values generated for each loudspeaker amplitude by f.i. a panning system commonly known-in-the-art, and outputs a resulting signal to the summation. The processing that the multiplier multiplies a signal by a prescribed coefficient may be referred to as ‘three-dimensional panning processing’. That is, the controller may give the related coefficient proper values corresponding to the respective output systems so that the resulting audio signal that is provided to the subject by means of the plurality of loudspeakers, has a dimensional shape and optionally a density and a position in space, e.g. an angle, distance, depth and height in relation to the subject. As a result of the processing of the multipliers, the sound is simulated properly for the propagation of direction and dimensions from the virtual sound source to the subject. The summations supply audio output signals of the multipliers to the respective output lines, each associated with a loudspeaker in a loudspeaker configuration shape.

Each output line may further comprise a signal attenuator having as attenuation coefficient:

a=1/N²
where N is the number of audio signal components yₙ in the signal distribution matrix and the obtained attenuation for a translates to gain G in decibels dB as

G(dB)=10 log₁₀(a)

It should be understood that the modification of input audio signal into audio signal components into loudspeaker audio signals, x->y_n->z_k, may be the process of a pre-calculated shape of a virtual sound source, and/or a shape that is transformed in real-time, i.e. the shape, size and density and/or the position and rotation of the shape in space are subject to changes in real-time generated by a controller, a pre-automated set of data executed in real-time and/or a real-time computer generated process.

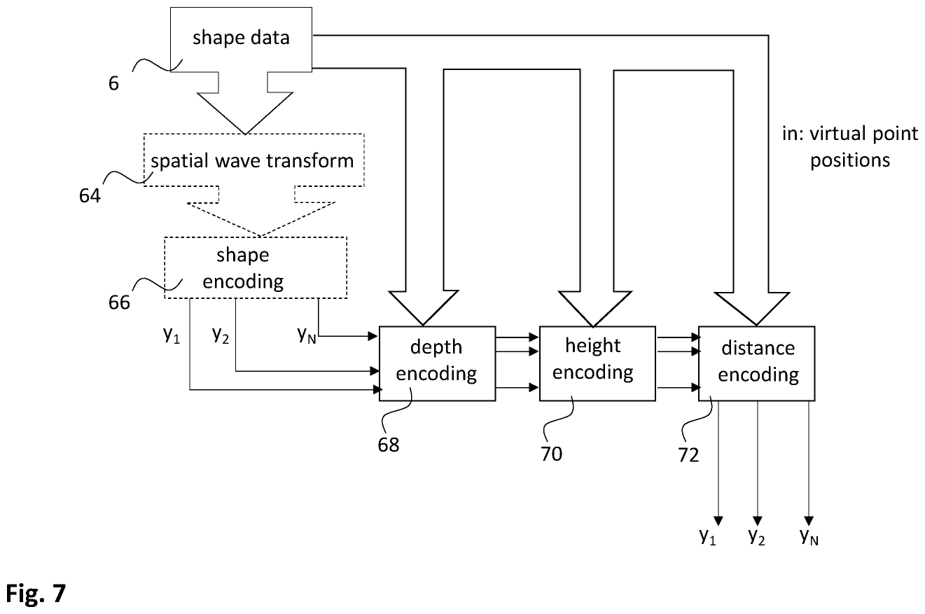

FIG. 7 illustrates a method according to an embodiment for determining the audio signal components 26 associated with respective virtual points defining the virtual sound source 10. In this embodiment, the method comprises obtaining shape data as virtual sound source information 6. The shape data defines the virtual points of the virtual sound source 10 that is to be perceived by the subject 2 upon hearing the audio signal.

In such embodiment, the method comprises a spatial wave transform 64, which means that, for the determination of each audio signal component, the input audio signal x(t) is modified to obtain a modified audio signal component using a signal delay operation introducing a time delay and determining the audio signal component based on a combination, e.g. a summation, of the input audio signal, or of an inverted and/or attenuated or amplified version of the input audio signal, and the modified audio signal component. The formula for determining the time delay that is introduced for determining the modified audio signal component may be given by

Δt=Vxₙ/v

wherein V is the dimensional volume of the shape and xₙ denotes for point n on the virtual shape a coefficient, each point having a relative spatial position denoted in Cartesian coordinates (xyz); and v is a constant relating to the speed of sound through a medium. The determination of the audio signal components by means of a spatial wave transform is also described in patent applications NL2024434 and NL 2025950 which contents should be considered included in this disclosure in their entirety.

It should be appreciated that the determination of a plurality of audio signal components respectively associated with virtual points of a virtual sound source may be referred to as shape encoding 66.

The obtained audio signal components associated with the respective virtual points of the virtual sound source may be further modified by what is referred to as depth encoding 68, height encoding 70 and distance encoding 72 in FIG. 7. These additional modifications may be performed in dependence of the virtual positions (xyz) of the virtual points with respect to the subject 2.

It should be understood that embodiments described herein may be performed in alternative order and using process flow that differ from those that are illustrated; and, that not all steps are required in every embodiment. In other words, one or more steps may be omitted or replaced, performed in different orders, in parallel with one another and/or additional steps may be added.

FIG. 8A is a flow chart illustrating a method and system for determining for determining an audio signal component for a virtual point of a virtual sound source 10. Determining an audio signal component for all virtual points may be referred to as performing a spatial wave transform, which is optionally performed. It should be appreciated that this method is performed for each individual virtual point of a virtual sound source in order to obtain all audio signal components for the respective virtual points defining the virtual sound source. The method for determining audio signal components is also described in patent applications NL2024434 and NL 2025950 which contents should be considered included in this disclosure in their entirety. Further, it should be appreciated that the flow chart of FIG. 8A can be replaced by any of the flow charts depicted in FIGS. 8D, 8E, 8F.

In the depicted embodiment, the input audio signal x(t) is modified (see lower branch of FIG. 8A) in order to obtain a first modified audio signal component. This modification of the input audio signal x(t) optionally comprises a signal inverting operation 74, comprises a signal delay operation 75 introducing a time delay and comprises a signal feedback operation 73 as shown. The time delay used in the signal delay operation 75 may be determined in accordance with the formula for determining the time delay as described above in relation to FIG. 7. In the depicted embodiment, the signal that is fed back is attenuated as shown by the amplifier 76 having a gain smaller than 1. Then, the first modified audio signal component is combined, see the summation 78, with the input audio signal in order to obtain a second modified audio signal component. Furthermore the second modified audio signal is further modified by an attenuation operation 79 and, optionally, a high-pass filter operation 80 to obtain a audio signal component y(t)_n associated with a virtual point of the virtual sound source 10.

The attenuation operation 79 after the summation operation 78 may comprise decreasing the gain G of the audio signal with −6 dB. The cut-off frequency f_{c} for the high pass filter in dependence of point n on a virtual shape may be determined as

f_{c}=v/V2(1−rₙ/R) for rₙ/R≤0.5

f_{c}=v/V2(rₙ/R) for rₙ/R>0.5

where v is a constant relating to the speed of sound through a medium, V is the dimensional volume of a virtual shape, rₙ denotes the spherical radius from the center of a virtual shape to point n, and R denotes the spherical radius from the center of the shape passing through the vertices where two or more edges of a virtual shape meet. In case of two or more values for R, the largest value R is considered.

FIG. 8B shows how a time delay operation, a signal inverting operation and a signal attenuation performed on a signal x(t) influence the signal. FIG. 8B shows an audio input signal x(t) with on the vertical axis amplitude and on the horizontal axis time; and a modified audio signal that has been inverted with respect to the audio input signal and time delayed by Δt and attenuated by a factor b.

FIG. 8C illustrates a method and system for determining an audio signal component associated with a virtual point according to an embodiment. In this example, an audio input signal x(t) is modified to obtain a first modified audio signal component using a signal delay operation introducing a first time delay Δtn.1 associated with a point on a virtual shape. Further, the audio input signal x(t) is modified to obtain a second modified audio signal using a signal delay operation introducing a second time delay Δtn.2 associated with the same point on the virtual shape. In the same way, more than two or many modified audio signal components may be obtained associated with one and the same point on a virtual shape. In FIG. 8C the number of modified audio signal components is indicated by ‘P’.

Furthermore, a modified signal component y(t)ₙ is obtained comprising a summation, e.g. combination of first, resp. second modified audio signal components, an attenuation operation in dependence of the number of modified audio signal components associated with one and the same point on a virtual shape, where

a=1/P², and

G(dB)=10 log₁₀(a)

and, optionally, a high-pass filter operation using the formula as described above with respect to obtaining the cut-off frequency f_{c} for the high pass filter in dependence of point n on a virtual shape.

It should be appreciated that the flow chart of FIG. 8C may be replaced by any of the flow charts depicted in FIG. 8G, 8H, 8I. Further, FIGS. 8C, 8G, 8H, 8I comprise repetitive parts (indicated by the dashed boxes). It should be appreciated that any of the flow charts depicted in FIGS. 8D, 8E, 8F can be used as repetitive part instead in FIGS. 8C, 8G, 8H, 8I.

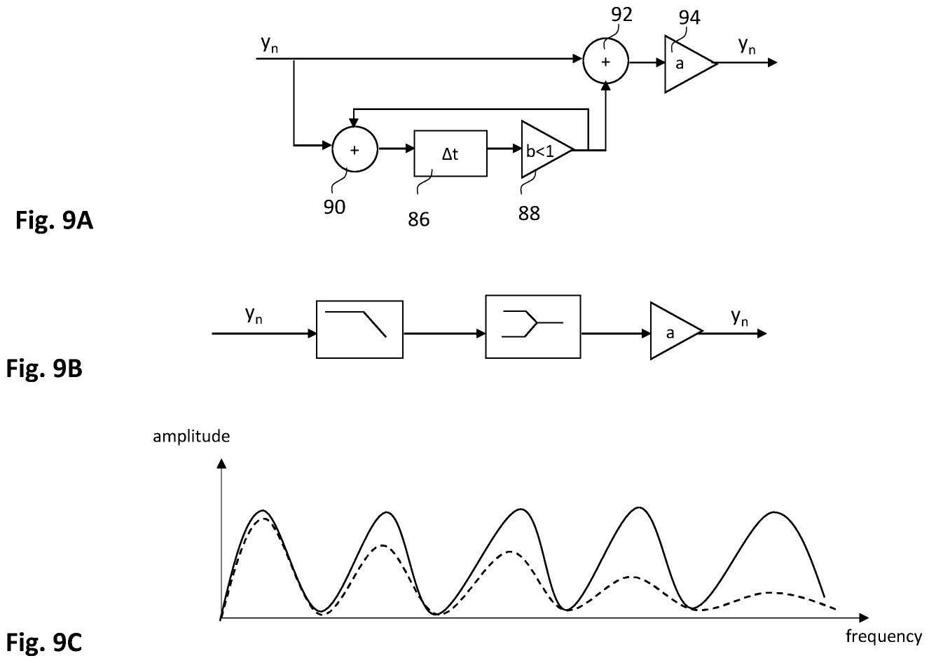

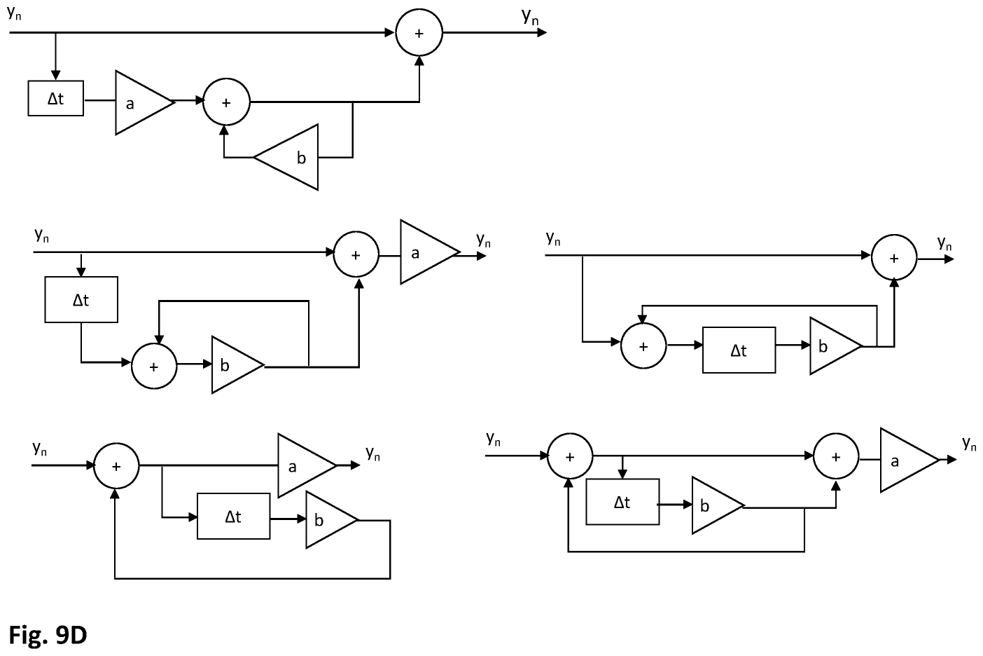

FIG. 9A is a flow chart illustrating a method for adding depth characteristics to an audio signal component. Such audio signal component y_n may be obtained for example in accordance with the flow charts depicted in FIG. 8A or FIG. 8C.

Adding the depth characteristics to the audio signal component in FIG. 9A comprises modifying the audio signal component y_n in question using a time delay operation 86 introducing a time delay, a signal attenuation 88 and a signal feedback operation 90 in order to obtain a modified version of the audio signal component and combining 92 the modified version of the audio signal component with the audio signal component in question. The signal attenuation 88 is performed in dependence of the virtual depth below the subject of the virtual point associated with the audio signal component in question.

In this embodiment, the signal attenuation is defined by parameter “b”. If value b=0 no depth of the virtual point below the subject will be encoded, if value b=1, a maximum depth for the virtual point associated with the audio signal component will be encoded.

The value “a” with which the result of the combination of modified audio signal and input audio signal is optionally attenuated or amplified 94 equals to

a=(1−b)x

where x is a multiplication factor to correct the signal gain G depending on the amount of signal feedback b that influences the steepness of a high-frequency dissipation curve. By varying value b, preferably between 0-1, a change in depth is added to the audio signal.

Preferably, the time delay Δt that is introduced by the time delay operation is as short as possible, e.g. shorter than 0.00007 seconds, preferably shorter than 0.00005 seconds, more preferably shorter than 0.00002 seconds. Most preferably, approximately 0.00001 seconds. In case of a digital sample rate of 96 kHz, the time delay may be 0.00001 seconds.

It should be appreciated that the flow chart of FIG. 9A may be replaced by any of the flow charts depicted in FIG. 9D.

FIG. 9B describes an embodiment of the invention, a signal processing module and/or code portion for depth encoding; comprising modifying the audio signal component y_n to obtain a further modified audio signal by using a low-pass filter operation, a low-shelf filter operation and an attenuation operation; where the cut-off frequency f_{c} of the low-pass filter, the cut-off frequency f_{c} and gain G of the low-shelf filter are variables dependent on the relative depth.

FIG. 9C shows an audio signal component (solid line) with on the vertical axis amplitude and on the horizontal axis frequency; and the audio signal component to which depth characteristics have been added (dashed line), the effect of which is shown by the gradual dissipation of the high-frequency energy compared to the low-frequency energy.

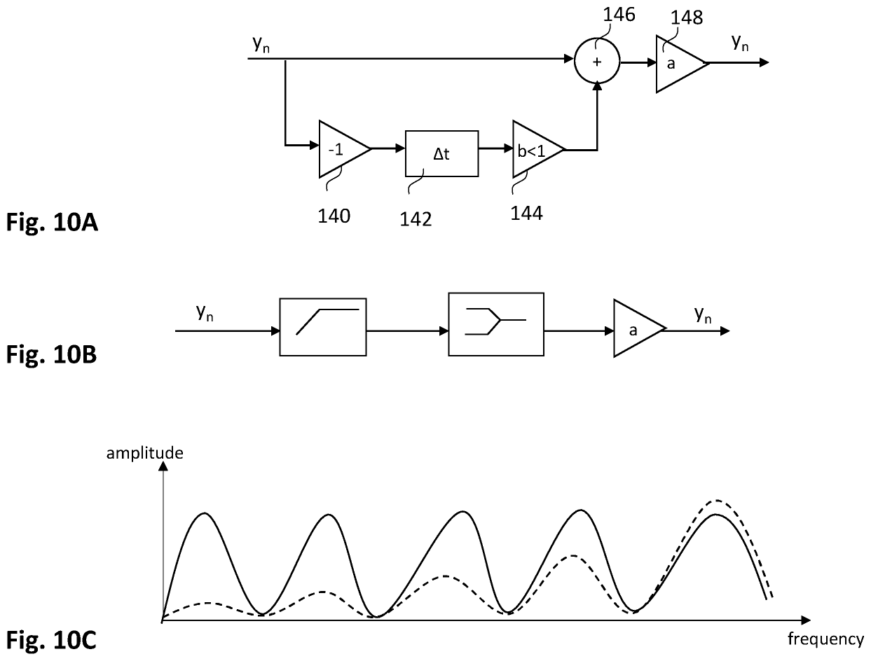

FIG. 10A is a flow chart illustrating a method for adding height characteristics to an audio signal component y_n that is associated with a virtual point positioned at a height above the subject. Adding the height characteristic to the audio signal component comprises modifying the audio signal component in question using a signal inverting operation 140, a signal delay operation 142 introducing a time delay and a signal attenuation 144 to obtain a modified version of the audio signal component and combining 146 the modified version of the audio signal component with the audio signal component in question. Herein the signal attenuation 144 is performed in dependence of the virtual height of the virtual sound source.

In this embodiment, if value b=0 no height characteristics will be added to the audio signal component. If value b=1, a maximum height of the virtual point will be perceived. If the first attenuation operation is performed, the gain G of value “a” of attenuation 148 may be equal to

a=(1−b)x

where x is a multiplication factor to correct the signal gain G depending on the amount of attenuation b that influences the steepness of a low-frequency dissipation curve. By varying value b, preferably between 0-1, a change in height can be added to an audio signal component.

Preferably, the time delay Δt that is introduced by the time delay operation 142 is as short as possible, e.g. shorter than 0.00007 seconds, preferably shorter than 0.00005 seconds, more preferably shorter than 0.00002 seconds. Most preferably, approximately 0.00001 seconds. In case of a digital sample rate of 96 kHz, the time delay may be 0.00001 seconds.

FIG. 10B describes an embodiment of the invention, a signal processing module and/or code portion for height encoding; comprising modifying the audio signal component y_n to obtain a further modified audio signal by using a high-pass filter operation, a high-shelf filter operation and an attenuation operation; where the cut-off frequency f_{c} of the high-pass filter, the cut-off frequency f_{c} and gain G of the high-shelf filter are variables dependent on the chosen height.

FIG. 10C shows audio signal component (solid line) with on the vertical axis amplitude and on the horizontal axis frequency; and the audio signal component to which height characteristics have been added (dashed line), the effect of which is shown by the gradual dissipation of the low-frequency energy compared to the high-frequency energy.

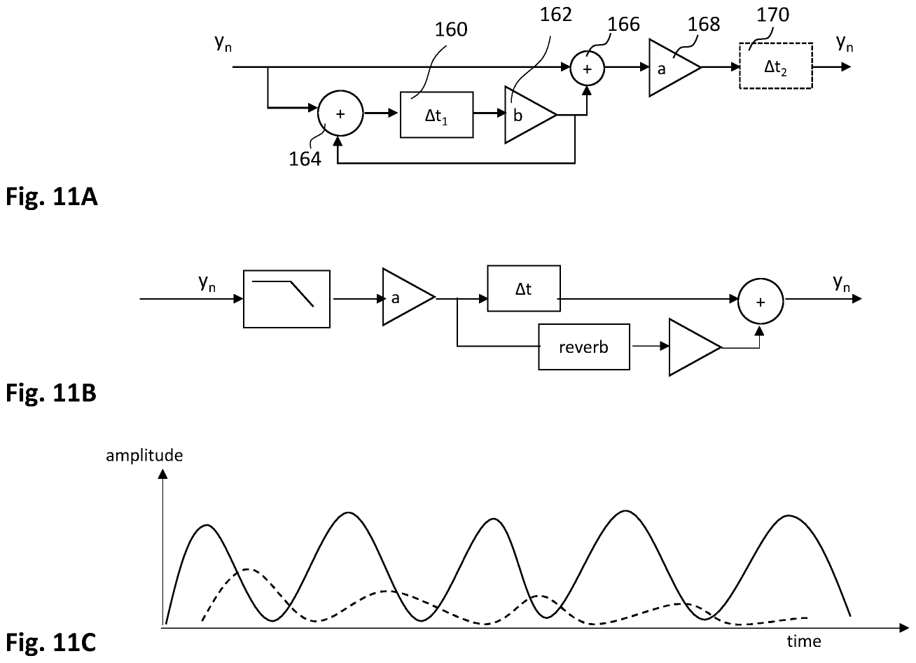

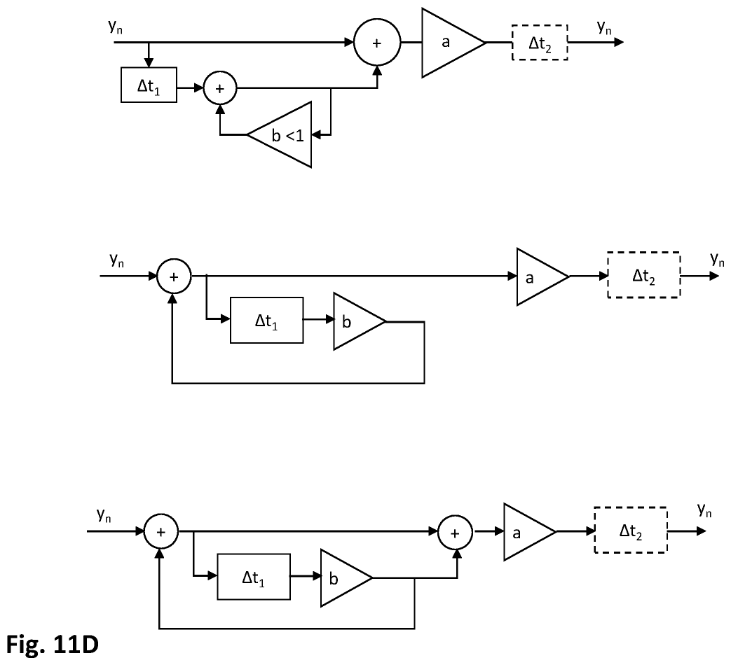

FIG. 11A is a flow chart illustrating a method for adding distance characteristics to an audio signal component y_n. Adding distance characteristics to the audio signal component comprises modifying the audio signal component in question using a first signal delay operation 160 introducing a first time delay, a first signal attenuation operation 162 and a signal feedback operation 164 in order to obtain a first modified version of the audio signal component and combining 166 the first modified version of the audio signal component with the audio signal component in question to obtain a second modified version of the audio signal component and performing a second signal attenuation 168 and optionally a second signal delay operation 170 introducing a second time delay on the second modified version of the audio signal component. Herein, the first 162 and second 168 signal attenuation are performed in dependence of the virtual distance from the subject.

In dependence of the distance of the virtual point associated with the audio signal component in question the values for b, the attenuation constant for operation 162, and the value for a, the attenuation constant for operation 168, is varied. The constants may be understood to indicate a constant with which a signal is multiplied. Thus, if such value is larger than 1, then a signal amplification is performed. If such value is smaller than 1, then a signal attenuation is performed. When b=0 and a=1 no distance will be encoded and when b=1 and a=0 a maximum distance will be encoded. The gain G of value a may relate to the value for b as

a=(1−b)x

where the value for x is a multiplication factor applied to the amount of signal feedback that influences the steepness of a high-frequency dissipation curve.

Preferably, the time delay Δt1 that is introduced by the time delay operation 160 is as short as possible, e.g. shorter than 0.00007 seconds, preferably shorter than 0.00005 seconds, more preferably shorter than 0.00002 seconds. Most preferably, approximately 0.00001 seconds. In case of a digital sample rate of 96 kHz, the time delay may be 0.00001 seconds

The optional time delay Δt2 that is introduced by the time delay operation 170 creates a Doppler effect associated with movement of the virtual sound source. The time delay may be determined as

Δt2=r/v

wherein r is the distance between the position of virtual point associated with the audio signal component in question denoted in Carthesian coordinates (xyz) and the subject, which may be expressed as a vantage point (xyz) and v a constant expressing the speed of sound through a medium.

It should be appreciated that the flow chart of FIG. 11A may be replaced by any of the flow chart depicted in FIG. 11D.

FIG. 11B describes an embodiment of the invention, a signal processing module and/or code portion for distance encoding; comprising modifying the audio signal component y_n to obtain a further modified audio signal by using a low-pass filter operation, an first attenuation operation and a time delay operation introducing a time delay. Optionally, a second modified audio signal is obtained by modifying the filtered and attenuated first modified audio signal by a reverb operation and a second attenuation operation, and a summation, e.g. combination of the first, resp. second modified audio signals to obtain a third modified audio signal y(t); where the cut-off frequency f_{c} of the low-pass filter, the gain G of the first, resp. second attenuation operations and the time delay Δt are variables dependent on the chosen distance.

FIG. 11C shows an audio signal component (solid line) with on the vertical axis amplitude and on the horizontal axis time; and the audio signal component to which characteristics have been added of distance increasing with time (dashed line), the effect of which is shown by the gradual decrease in amplitude of the modified audio signal compared to the audio input signal; and, the gradual increase in the time delay of the modified audio signal compared to the audio input signal.

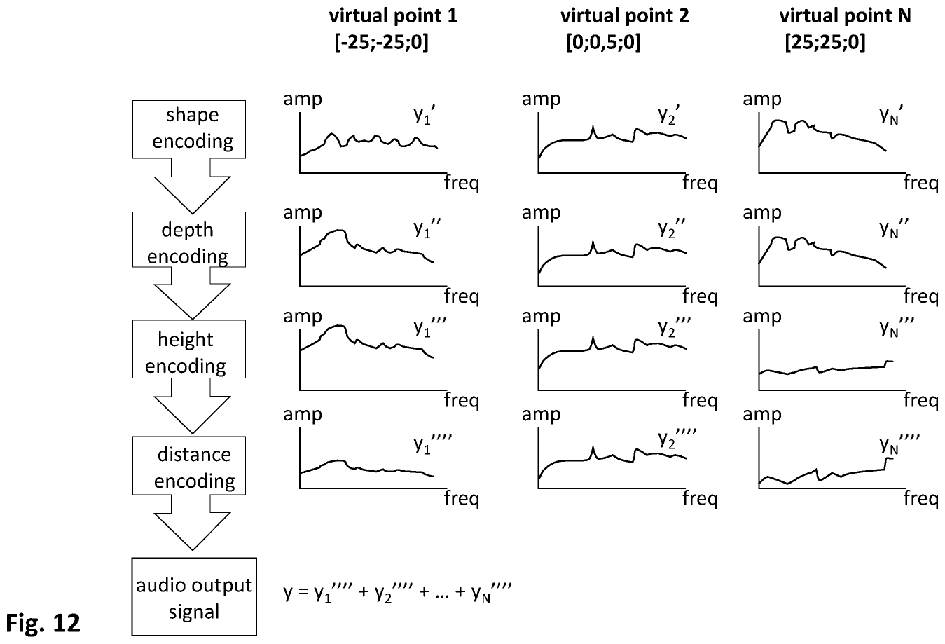

FIG. 12 describes an embodiment of the invention, where an input audio signal is modified by a shape encoding operation to obtain a modified audio signal component yₙ′; further modified by a depth encoding operation to obtain a modified audio signal component yₙ″; further modified by a height encoding operation to obtain a modified audio signal component yₙ′″; further modified by a distance encoding operation to obtain a modified audio signal component yₙ″″. The encoding operations for shape, depth, height and distance are performed in dependence of the position of yₙ (x, y, z) associated with a point on a virtual shape, and the dimensional volume of a virtual shape V; and/or in dependence of the position of yₙ (x, y, z) and the position of the subject, i.e. the observer which may be denoted by a vantage point (x, y, z). FIG. 12 schematically shows how for three virtual points “1”, “2”, and N”, the audio signal components are determined.

The resulting audio output signal is the summation of audio signal components yₙ″″ to obtain an audio signal with spectral modifications, such that it closely resembles the resonance of a sound source with a distinct shape; i.e. the projection of a virtual sound source with a dimensional shape, size and density at a particular distance, height and depth in relation to the subject, a ‘sound shape projection’.

The shape data used to obtain the modified audio signals y may be pre-calculated and stored on a readable digital or analogue storage medium; and, generated and/or modified in real-time and provided to the system as a data-streaming input. In another embodiment, the shape data comprises pre-recorded signals of a sounding object of a particular shape, size and material(s), captured at defined angle and distance to the object and describing attributes of the acoustic propagation of the object in space. In another embodiment, the shape data comprises of the acquired spectral modification data of a sound signal originating from a sounding object of particular shape, size and material(s), captured at a defined angle and distance, i.e. the ratio of amplitudes between all frequencies or frequency bands that are attributes of the acoustic propagation of the object in space.

In an embodiment of the invention, the audio signal processing and/or code portions used in the invention may include other methods known-in-the-art to obtain modified audio signal(s) and to encode (parts of) the shape data in the modified audio signal, including real-time FFT (Fast Fourier Transform) Analysis, Ray Tracing, Bandpass Filtering Synthesis and Convolution Synthesis. In another embodiment, the acquisition of shape data may be an input to a sound signal generating device and modify a generated audio signal, such as a sine-wave signal, by applying methods known-in-the-art, such as Additive Synthesis.

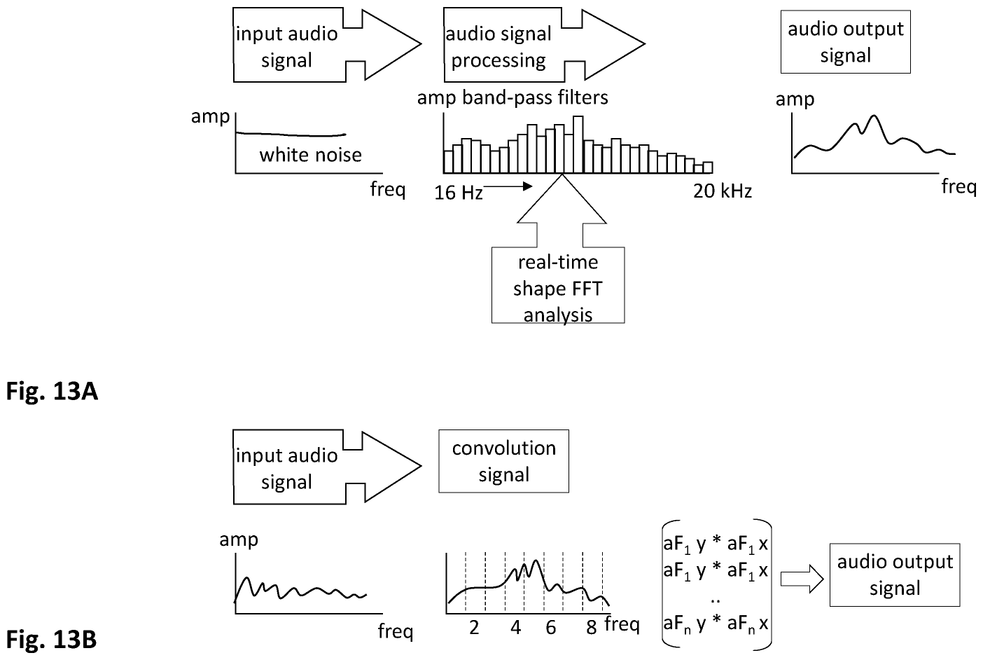

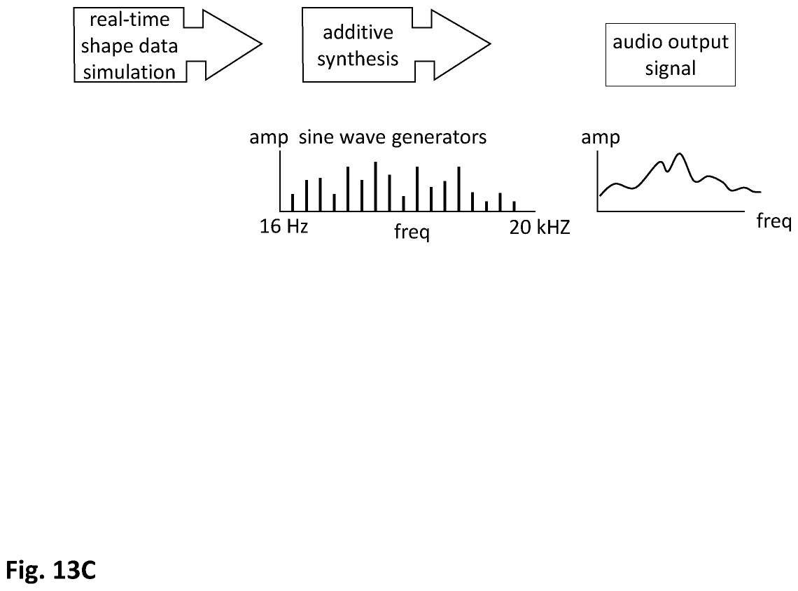

FIG. 13A describes an embodiment of the invention, a signal processing module and/or code portion for spatial encoding comprising modifying an input audio signal by modulating a set of band-pass filters for a chosen resolution of frequency bands in dependence of real-time and/or scripted FFT-analysis of a sound source's shape transformations in 3-dimensional space. The resulting audio output signal has encoded spectral modifications such that it resembles the resonance of a sound source with a distinct shape; i.e. the projection of a virtual sound source with a dimensional shape, size and density at a particular distance, height and depth in relation to the subject, a ‘sound shape projection’.

FIG. 13B describes an embodiment of the invention, a signal processing module and/or code portion for spatial encoding comprising an input audio signal and a convolution signal; where the convolution signal is a pre-recorded, time-based audio file of a sound source with a shape, captured from a particular position and at particular distance, height and depth from the source; and, modifying the input audio signal by a convolution operation for a chosen resolution of frequency bands. The resulting audio output signal has encoded spectral modifications such that it resembles the resonance of a sound source with a distinct shape; i.e. the projection of a virtual sound source with a dimensional shape, size and density at a particular distance, height and depth in relation to the subject, a ‘sound shape projection’.

FIG. 13C describes an embodiment of the invention, a signal generating module and/or code portion comprising real-time and/or scripted shape data and/or spatial simulation data to modify the amplitude of a chosen resolution of sine wave generators with a fixed frequency, an additive synthesis operation; The resulting audio output signal has encoded spectral modifications such that it resembles the resonance of a sound source with a distinct shape; i.e. the projection of a virtual sound source with a dimensional shape, size and density at a particular distance, height and depth in relation to the subject, a ‘sound shape projection’.

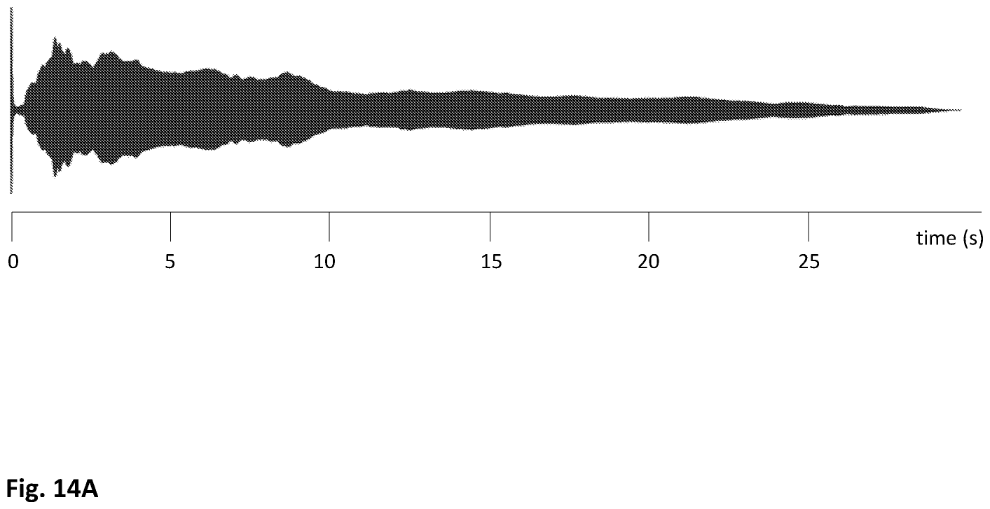

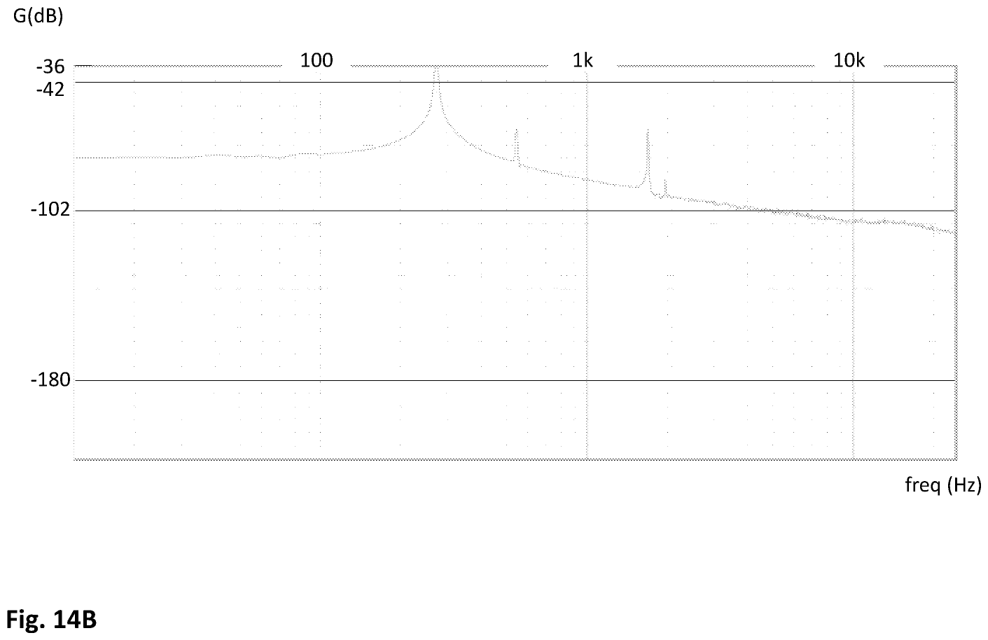

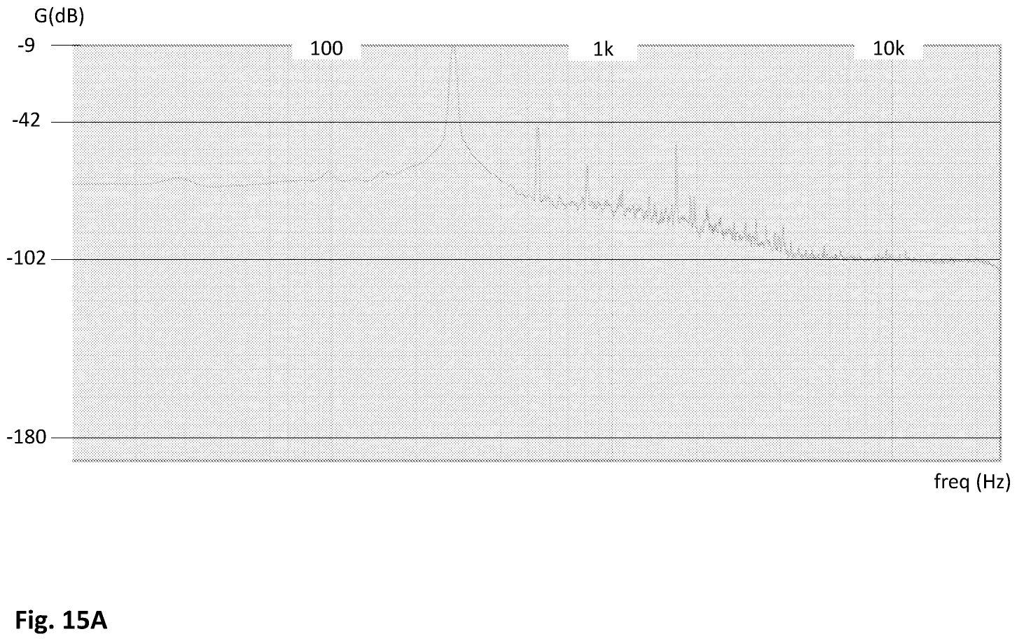

FIG. 14A shows the audio signal of an unweighted tuning fork recorded in a sound recording studio. The audio signal shows a total sustain in amplitude from the attack of the tone to finish in ˜30 sec. The signal is characterized by a short attack and decay during resp. the first 0.5 seconds of the audio signal, and a long sustain and release, during resp. 0.5-30 sec of the audio signal.

In an embodiment of the invention, the audio input signal may be one or several musical tones or rhythmic pulsations, i.e. a sound signal with steady periodic oscillation, a ‘pitch’ or ‘pulse’; and a ‘timbre’, meaning a distinguishable structure of higher-order harmonics to a fundamental pitch which are present in the sound and characterize the sound source. In an embodiment, a musical tone, such as obtained by a unweighted tuning fork, has been recorded in a studio to obtain the audio signal that is input for the system to improve physiological condition of a subject as described herein. It should be understood that the invention may include the use of other audio input signals of any other character and time duration, originating from other sound sources and/or obtained by any other means, including the repetition of the signal and various time exposures of the listener to (repetitions of) such audio signal.

The conducted experiments referred to in this disclosure have been obtained with sound stimuli of an unweighted tuning fork as the audio input signal. The musical tone of the tuning fork is repeated several times in its entirety during a total exposure period of 5 minutes.

FIG. 14B shows a mean spectrograph of 0.5-30 sec of an unweighted tuning fork on 272.2 Hz (fundamental pitch ˜C #) with distinguishable harmonics on 544.4 Hz (1:2 frequency ratio, 1st octave of the fundamental pitch ˜C #) and 1701.25 Hz (6.25:1). The power ratio of the fundamental to the first harmonic is 1:0.0015 (−28 dB) and from the fundamental to the second harmonic 1:0.0015 (−28 dB). The signal as depicted in FIG. 14B was used as input audio signal for tasks 1-4, as described in further detail below.

Several experiments have been conducted to test subjects' responses to an audio signal as described herein. The experiments involved four “tasks”, referred to as task 1, task 2, task 3 and task 4. Task 1 involved providing subjects a reference signal that does not project a virtual sound source as described herein (see FIG. 5E). Task 2 involved providing an audio signal associated with a virtual sound source shaped as a pyramid (see FIG. 5B). Task 3 involved providing an audio signal associated with a virtual sound source shaped as a cube (see FIG. 5C). Task 4 involved providing an audio signal associated with a virtual sound source shaped as a sphere (see FIG. 5D).

The exact parameters used in the flow charts of FIGS. 9A, 10A, 11A and 6 for generating the audio signals associated with task 2-4 (see FIG. 5F), were determined as follows.

The values for Δt, a, and b in building blocks FIG. 9A and FIG. 10A are obtained as follows: Delay time Δt for operation 86 in FIG. 9A is as small as possible but >0, see explanation above with FIGS. 9A and 10A. The sample rate of performing task 1-4 was 48 000 Hz, thus Δt-0.02 ms.

[y] represents the vertical axis, i.e. height, in the Cartesian coordinates (x,y,z) of a virtual point n. If [y]ₙ<0 then depth Dₙ is defined as Dₙ=y/−1 and height [y]ₙ=0; if [y]ₙ>0 then height Hₙ is defined as Hₙ=y and Dₙ=0.

The coefficient b for operation 88 in FIG. 9A is obtained as b=(1/D_{O})Dₙ
where D_{O} is the threshold depth (in m) and the attenuation gain G(b) in dB is given by

G(b)=10 log₁₀(P_{b}/Pₒ)
where P_{b} is the power value b and Pₒ is the reference power Pₒ=1

The coefficient a for operation 94 in FIG. 9A is obtained as a=(1−b) x, where x is a multiplication factor, in the case of performing task 2-4 set to a value of x=0.75
and the attenuation gain G(a) in dB is given by

G(a)=(10 log₁₀(Pₐ/Pₒ))x
where Pₐ is the power value a and Pₒ is the reference power Pₒ=1

The coefficient b for operation 144 in FIG. 10A is obtained as

b=(1/H_{O})Hₙ
where H_{O} is the threshold height (in m) and the attenuation gain G(dB) is given by

G(b)=10 log₁₀(P_{b}/Pₒ)
where P_{b} is the power value b and Pₒ is the reference power (Pₒ=1)

The coefficient a for operation 148 in FIG. 10A is obtained as a=(1−b) x, where x is a multiplication factor, in the case of performing task 2-4 set to a value of x=0.115
and the attenuation gain G(a) in dB is given by

G(a)=(10 log₁₀(Pₐ/Pₒ))x
where Pₐ is the power value a and Pₒ is the reference power Pₒ=1

The values for Δt1, a, b, and Δt2 in building blocks 160, 168, 162, 170, respectively in FIG. 11A are obtained as follows:

Delay time Δt1 is as small as possible but >0, see explanation at FIGS. 9A and 10A. The sample rate of performing task 1-4 was 48 000 Hz, thus Δt1˜0.02 ms.

The coefficient b for operation 162 in FIG. 11A is obtained as b=(1/r_{O}) r_{O->n}
where r_{O} is the threshold distance (in m),
distance r_{O->n} between the observer O (0,0,0) and virtual point n (x,y,z) is defined as

r_{O->n}=√((xₙ−x_{O})²+(yₙ−y_{O})²+(zₙ−z_{O})²)
and the attenuation gain G(b) in dB is given by

G(b)=10 log₁₀(P_{b}/Pₒ)
where P_{b} is the power value b and Pₒ is the reference power Pₒ=1

The coefficient a for operation 168 in FIG. 11A is determined as

a=Pₒ(1/r_{O->n})x
where Pₒ is the reference power level and the obtained coefficient a translates to gain G(a) in dB as

G(a)=(10 log₁₀(Pₐ/Pₒ))x
and where x is a multiplication factor, in the case of performing task 2-4 set to a value of x=1.1 Delay time Δt2 for operation 170 in FIG. 11A is obtained by

Δt2=r_{O->n}/v
where v is the propagation speed of sound travelling through a medium, in the case of task 2-4 set to 343 m/sec, i.e. the speed of sound through air at average temperature of 20 C and humidity of ca 50%.

The loudspeaker coefficients a (yₙ->zₙ) in building block 13 in FIG. 6 are obtained using a panning algorithm, in the case of performing task 1-4 the following panning algorithm was used:

A loudspeaker configuration (see FIG. 5A) is divided into loudspeaker configuration shapes, consisting of projection planes and volumes as

<speaker speakerType=“satellite” z=“0” y=“1.26” x=“0” ch=“1” id=“A”/>
<speaker speakerType=“satellite” z=“0.59” y=“0.41” x=“−1.02” ch=“2” id=“B”/>
<speaker speakerType=“satellite” z=“0.59” y=“0.41” x=“1.02” ch=“3” id=“C”/>
<speaker speakerType=“satellite” z=“−1.18” y=“0.41” x=“0” ch=“4” id=“D”/>
<speaker speakerType=“satellite” z=“1.18” y=“−0.41” x=“0” ch=“5” id=“E”/>
<speaker speakerType=“satellite” z=“−0.59” y=“−0.41” x=“1.02” ch=“6” id=“F”/>
<speaker speakerType=“satellite” z=“−0.59” y=“−0.41” x=“−1.02” ch=“7” id=“G”/>
<speaker speakerType=“satellite” z=“0” y=“−1.26” x=“0” ch=“8” id=“H”/>
<-- Top Layer -- >
<shape speakers=“D B A” type=“projectionTriangle”/>
<shape speakers=“B C A” type=“projection Triangle”/>
<shape speakers=“C D A” type=“projection Triangle”/>
<-- Mid Layer -- >
<shape speakers=“E C B” type=“projectionTriangle”/>
<shape speakers=“E F C” type=“projectionTriangle”/>
<shape speakers=“F D C” type=“projection Triangle”/>
<shape speakers=“F G D” type=“projectionTriangle”/>
<shape speakers=“G B D” type=“projectionTriangle”/>
<shape speakers=“G E B” type=“projectionTriangle”/>
<-- Bottom Layer -- >
<shape speakers=“H E G” type=“projection Triangle”/>
<shape speakers=“H F E” type=“projectionTriangle”/>
<shape speakers=“H G F” type=“projectionTriangle”/>
<-- Six Tetrahedrons filling the inside -- >
<shape speakers=“F E C D” type=“tetrahedron”/>
<shape speakers=“G E D B” type=“tetrahedron”/>
<shape speakers=“B D C E” type=“tetrahedron”/>
<shape speakers=“F G E D” type=“tetrahedron”/>
<shape speakers=“G F E H” type=“tetrahedron”/>
<shape speakers=“C D B A” type=“tetrahedron”/>
<projectionPoint z=“0” y=“2” x=“0”/>
</grid>
<routing value=“1 2 3 4 5 6 7 8”/>
<center z=“0” y=“0” x=“0”/>
</setup>

If a point associated with yₙ is located at a projection angle of a loudspeaker projection plane, or is located within a loudspeaker volume, consisting of loudspeakers zₙ on a particular face within a particular volume of the loudspeaker configuration, then for all loudspeakers not contained in the loudspeaker configuration shape

a(yₙ->zₙ)=0

and for each loudspeaker located on the loudspeaker configuration shape the distance r_{N} for yₙ->xₙ is determined as

rₙ=√((x_{yn}−x_{zn})²+(y_{yn}−y_{zn})²+(z_{yn}−z_{zn})²)

Furthermore

Σr=r₁+r₂+ . . . +rₙ

and the amplitude of signal yₙ for each loudspeaker zₙ is determined as

a(yₙ->zₙ)=1/(Σr/rₙ)

and the attenuation gain in dB is determined as

G(yₙ->zₙ)=10 log₁₀(Pₐ/Pₒ)

and thus

yₙ(a)=Σa(yₙ->zₙ)=1

which yields equal power panning.

The attenuation a of each obtained loudspeaker signal zₙ in FIG. 6 is obtained as a=1/N² where N=number of points defined on the shape (task 1 N=2, task 2 N=14, task 3 N=26, task 4 N=18). For each task, the output level of all audio output signals fed to the loudspeakers was further manually attenuated or amplified to obtain the exact same sound pressure level at subject (measured acoustically with a dB meter device).

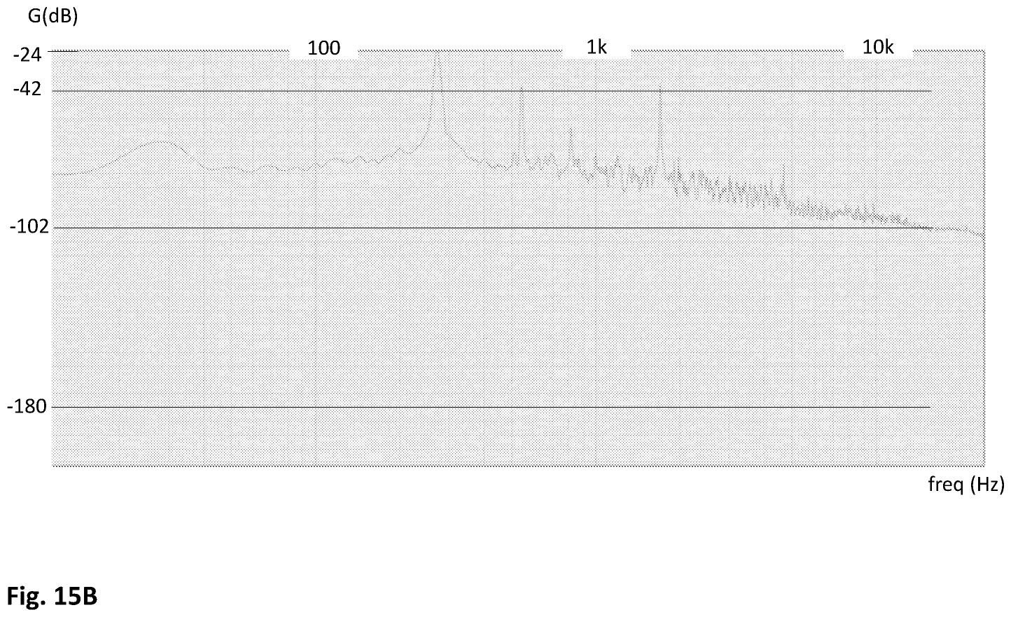

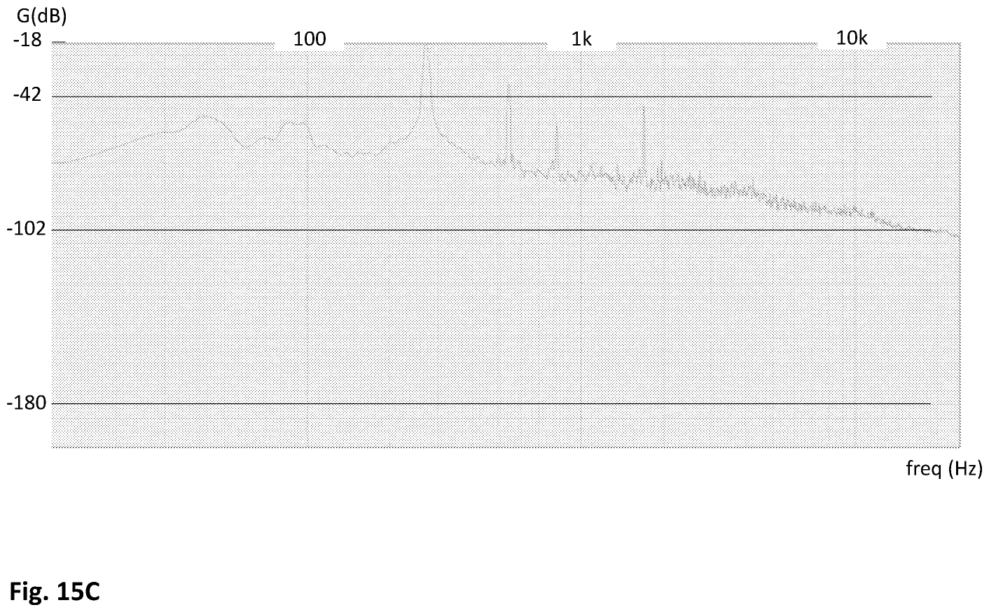

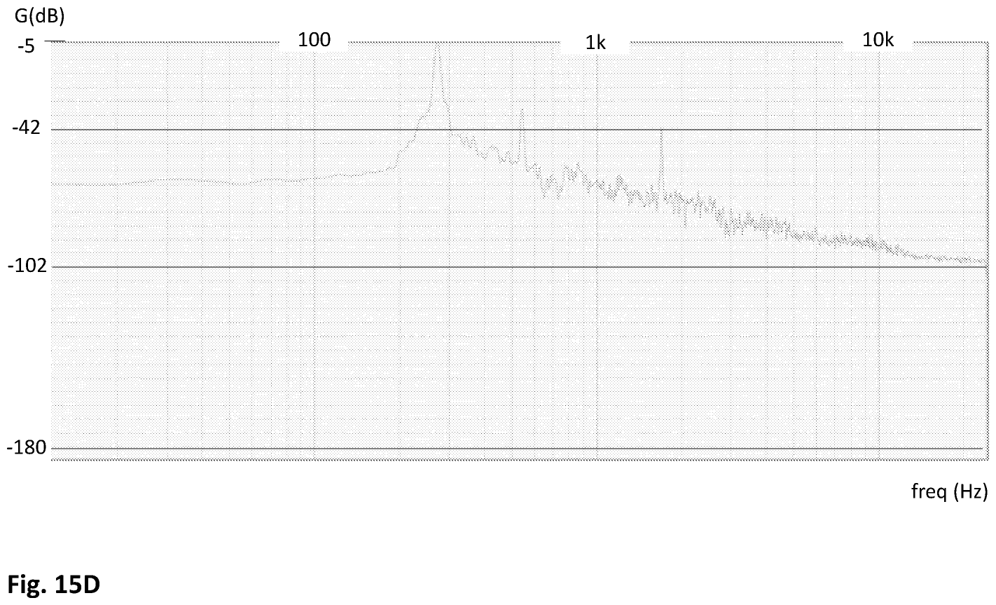

FIG. 15A shows a mean spectrograph of 0.5-30 sec of a recorded unweighted tuning fork, played back in stereo sound projection using high-fidelity loudspeakers in a sound-proofed, acoustically treated environment, also referred to as ‘task 1’. FIG. 5E shows the loudspeaker configuration that was used for task 1.

Compared to the input audio signal, the audio output signal comprising stereo sound projection shows an increase in power ratio of the fundamental to the first harmonic of 1:0.0003 (−35 dB) and from the fundamental to the second harmonic of 1:0.00008 (−41 dB).

By reproduction of the audio signal using a “standard” method, such as a stereo sound system, one may conclude that some of the recorded information is modified, i.e. the strength or presence of occurring harmonics in the spectrum of the recorded sound source is partially diminished or obscured by the propagation of the output medium.

FIG. 15B shows a mean spectrograph of 0.5-30 sec of an audio signal that has been generated with a recorded unweighted tuning fork as input audio signal. The generated audio signal was played back using high-fidelity loudspeakers in a sound-proofed, acoustically treated environment. In this embodiment, the virtual sound source is a pyramid. Providing this audio signal to a subject is also referred to as ‘task 2’. FIG. 5A shows the loudspeaker configuration used for task 2, FIG. 5B illustrates the virtual sound source that was used for task 2 and FIG. 5F shows the signal processing that was used for task 2.

Compared to the input audio signal shown in FIG. 14B, audio signal of FIG. 15B, which is provided to a subject, the subject, upon hearing this audio signal perceiving the audio signal as originating from a pyramid shaped virtual sound source, a significant decrease in power ratio of the fundamental to the first harmonic (2:1) can be observed of 1:0.025 (−16 dB) and from the fundamental to the second harmonic (6.25:1) of 1:0.04 (−14 dB). Furthermore, a third harmonic becomes distinguishable at 816.6 Hz (3:1) with a power ratio relating to the fundamental of 1:0.0004 (−34 dB).

FIG. 15C shows a mean spectrograph of 0.5-30 sec of an audio signal that has been generated with a recorded unweighted tuning fork as input audio signal. The generated audio signal was played back using high-fidelity loudspeakers in a sound-proofed, acoustically treated environment. In this embodiment, the virtual sound source is a cube. Providing this audio signal to a subject is also referred to as ‘task 3’. FIG. 5A shows the loudspeaker configuration used for task 3, FIG. 5C illustrates the virtual sound source that was used for task 3 and FIG. 5F shows the signal processing that was used for task 3.

Compared to the audio input signal (see FIG. 14B), in the audio signal comprising the sound shape projection of a cube (see FIG. 15C), a significant decrease in power ratio of the fundamental to the first harmonic (2:1) can be observed of 1:0.015 (−18 dB) and from the fundamental to the third harmonic (3:1) of 1:0.00025 (−36 dB). The power ratio of the fundamental to the second harmonic (6.25:1) is equal to the audio input signal 1:0.0015 (−28 dB).

FIG. 15D shows a mean spectrograph of 0.5-30 sec of a recorded unweighted tuning fork, played back as a sound shape projection using high-fidelity loudspeakers in a sound-proofed, acoustically treated environment. In this embodiment, the virtual sound source is a sphere, also referred to as ‘task 4’. FIG. 5A shows the loudspeaker configuration used for task 4, FIG. 5D illustrates the virtual sound source that was used for task 4 and FIG. 5F shows the signal processing that was used for task 4.

Compared to the audio input signal (see FIG. 14B), in the audio output signal comprising the sound shape projection of a sphere (FIG. 15D), the equal power ratio of the fundamental to the first harmonic (2:1) can be observed of 1:0.0015 (−28 dB) and an increase of power ratio from the fundamental to the third harmonic (3:1) of 1:0.00025 (−36 dB).

By reproduction of the audio signal using a sound shape projection, one may conclude that the recorded information is modified such that the resulting spectrum resembles the resonance of the projected shape, e.g. the resonance of a sound source with a shape of a pyramid, cube or sphere, and that the strength or presence of the occurring harmonics in the spectrum of the audio input source may be increased or decreased due to the shape projection, and that such sound shape projection (at least partially) overrules the spatio-spectral properties resulting from propagation of the output medium, i.e. the individual loudspeakers.

In an embodiment, a virtual sound source may be shaped as a pyramid, a cube or a sphere. Data with regards to the physiological response of the human body after exposure to the projection of such shapes as referred to in this disclosure, has been obtained with said projection of these three shapes as examples. These three shapes were chosen as fundamental basic geometries and their relation to natural processes (Y Li et al., 2015), crystallisation processes (C Park et al, 2010), and prior subject of physiological experiments (I R Kumar et al., 2005). The effects referred to as the effects of sound shapes refers to the observable effect that is obtained to a significant degree with either of these shapes, i.e. the general effect of attributing shape to sound, other than the effect of rhythm, pitch or timbral information which is commonly present in sound; and, that the accurate projection of shape has a distinct difference and/or increase of such effect in comparison to projection of the same audio signal while not taking specifically into account the shape of the projected sound object, f.i. using standard methods known in the art, such as stereo sound projection. Although the effects of each distinct shape referred to in this disclosure may show distinct differences, the claims on the method described herein refer to those observable and measurable effects that the projection of the shapes have in common. It should be understood that the method comprising the invention refers to the audible production of shape, and thus may include any geometrical and/or non-geometrical shape and/or mathematically coherent projections of shapes of any spatial dimensions, and/or shapes that transform in periodic oscillations, such as spirals.

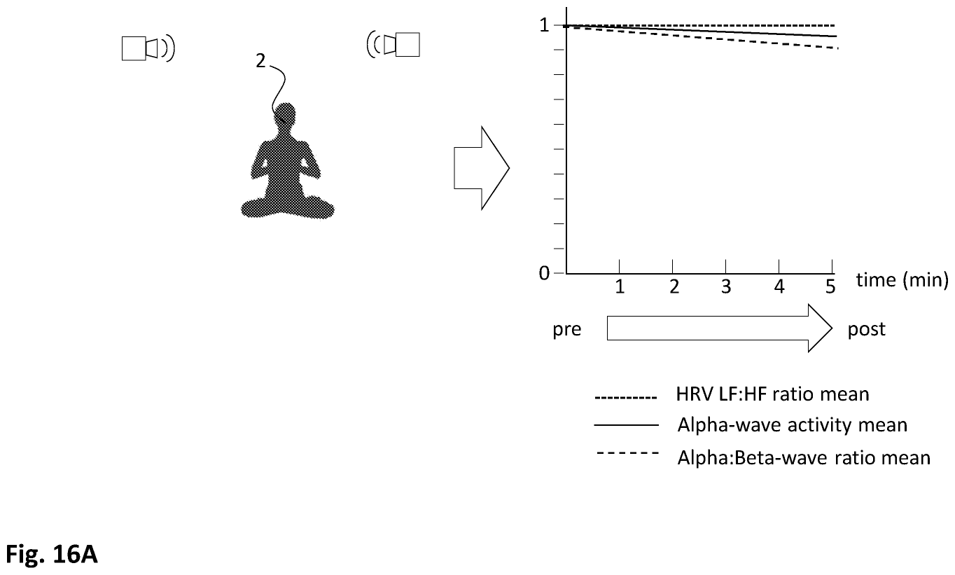

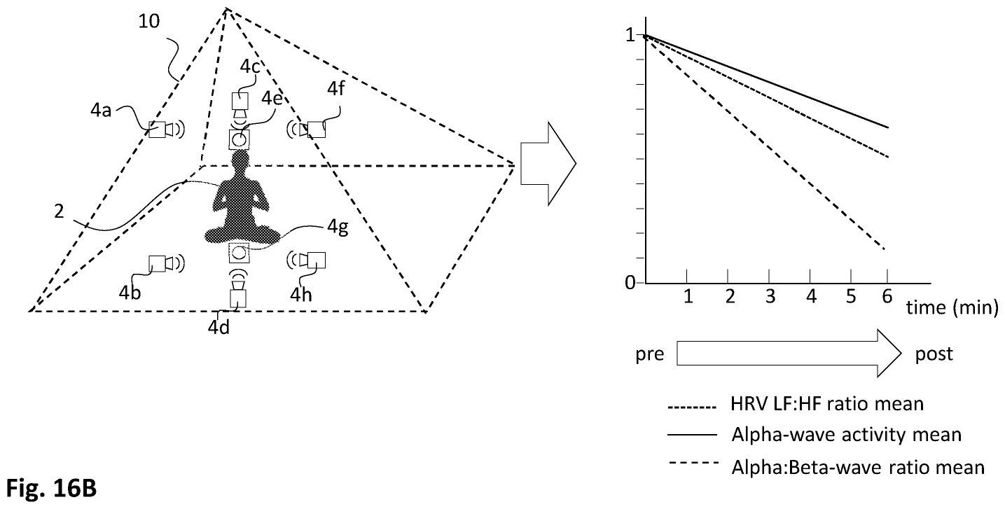

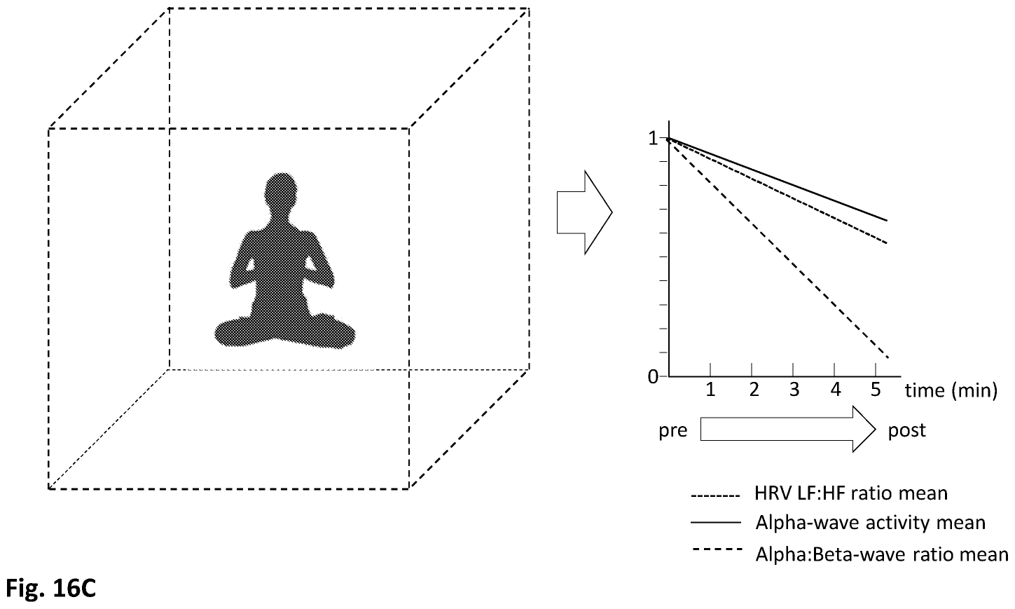

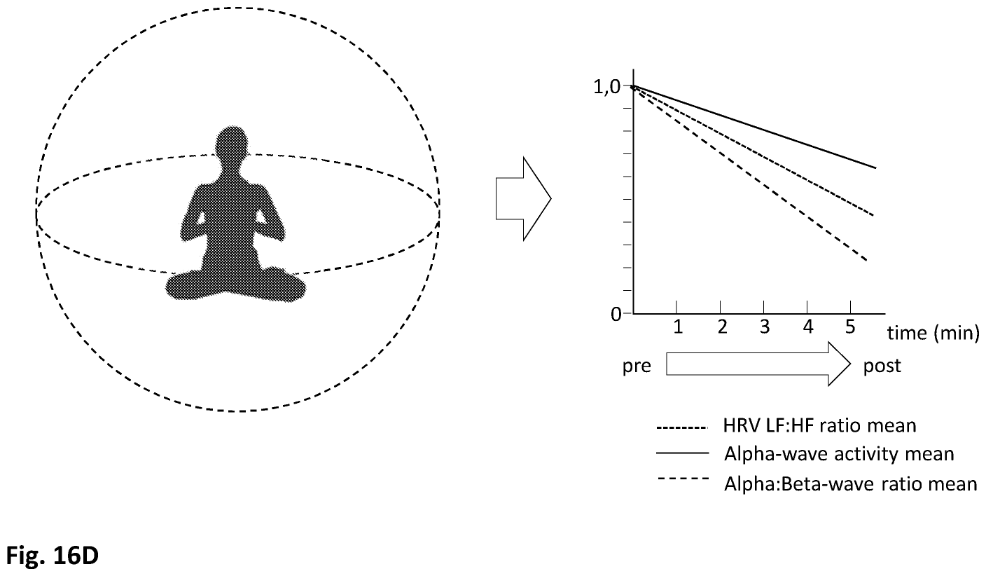

FIG. 16A shows a method for improving the physiological condition of a subject according to an embodiment. Herein, a subject 2 is positioned in the center of a stereo sound projection, i.e. ‘task 1’ (see FIG. 5E for the loudspeaker configuration and FIG. 15A for the provided audio signal), where the audio input signal is distributed equally left and right of the subject and at normalized sound pressure level measured from the subject's position. Note that for all tasks 1-4 the sound pressure level was the same.

On the right is shown the summary of the measured physiological effects observed after 5 minutes of the sound exposure. No distinguishable effect in the subject can be observed in the mean power of the Alpha-wave activity comparing post-exposure to pre-exposure, hereinafter also referred to as ‘base-condition’. The Alpha:Beta-wave ratio of the Brain Activity and the LF:HF ratio of the Heart-Rate Variability have slightly decreased comparing post-exposure to task 1 to base condition, but not decreased significantly.

FIG. 16B shows an embodiment placing a subject in the center of a sound shape projection of a pyramid, i.e. ‘task 2’ (see FIG. 5B for the virtual sound source and FIG. 15B for the provided audio signal), where the generated audio signal is distributed by loudspeakers placed left, right, above, below, front and back of the subject and at normalized sound pressure level measured from the subject's position.

On the right is shown the summary of the measured physiological effects observed after 5 minutes of the sound exposure. A significant decrease can be observed in Alpha-wave mean power and in the Alpha:Beta-wave power ratio of the Brain Activity; and, a significant decrease in LF:HF power ratio of the Heart Rate Variability; the measured effects of which indicate improved homeostasis of the subject.

FIG. 16C shows an embodiment placing a subject in the center of a sound shape projection of a cube, i.e. ‘task 3’ (see FIG. 5C for the virtual sound source and FIG. 15C for the provided audio signal), where the audio input signal is distributed is distributed by loudspeakers placed left, right, above, below, front and back of the subject and at normalized sound pressure level measured from the subject's position.

On the right is shown the summary of the measured physiological effects observed after 5 minutes of the sound exposure. A significant decrease can be observed in Alpha-wave mean power and a very significant decrease in the Alpha:Beta-wave power ratio of the Brain Activity; and, a significant decrease in LF:HF power ratio of the Heart Rate Variability; the measured effects of which indicate improved homeostasis of the subject.

FIG. 16D shows an embodiment placing a subject in the center of a sound shape projection of a sphere, i.e. ‘task 4’ (see FIG. 5D for the virtual sound source and FIG. 15D for the provided audio signal), where the audio input signal is distributed by loudspeakers placed left, right, above, below, front and back of the subject and at normalized sound pressure level measured from the subject's position.

On the right is shown the summary of the measured physiological effects observed after 5 minutes of the sound exposure. A significant decrease can be observed in Alpha-wave mean power and in the Alpha:Beta-wave power ratio of the Brain Activity; and, a significant decrease in LF:HF power ratio of the Heart Rate Variability; the measured effects of which indicate improved homeostasis of the subject.

Importantly, the observed effect of the sound shape projection as in task 2-4, is that all observed effects significantly increase compared to task 1 and compared to base condition; that is, the decrease in mean power of Alpha wave activity of the brain, the significant decrease in the difference ratio slope between Alpha:Beta-wave; and, the significant decrease in the difference ratio slope between LF:HF power ratio of the Heart Rate Variability.

Decrease in Alpha:Beta-wave power ratio may be interpreted as enhanced relaxation (International Medical Journal (1994) 23(no. 1):1-3⋅April 2016 & R F Navea et al, Conference paper-Project Einstein 2015, At De La Salle University—Manila) and indicative of enhanced cohesion in brain waves. Lower levels of Alpha-waves at the left front central were significantly associated with higher levels of self acceptance, environmental mastery, personal growth and total Psychological Well Being (H L. Urryet et al, Psychol Sci. 2004 June; 15(6):367-72) also suggesting a positive effect on cardiovascular and respiratory systems in accordance to mood induction (Matti Grohn et al, Proceedings of the 18th International Conference on Auditory Display, Atlanta, GA, USA, June 18-21, 2012). Decrease in Alpha-wave activity is also reported to relate to higher levels of oxygen in the blood (H Yuan et al, Neuroimage. 2010 Feb. 1; 49(3): 2596). Results indicate participants were in a state of enhanced concentration, i.e. immersion (S. Lim et al, Sensors (Basel) 2019 Apr. 8; 19(7):1669). Furthermore, while immersed in a VR experience, Alpha-wave activity has been shown to decrease during arithmetic tasks also suggesting inflicting attention inwards, compared to purely mental tasks (Elisa Magosso et al, Computational Intelligence and Neuroscience Volume 2019).

It is generally accepted that the activities of the autonomic nervous system (ANS), which consists of the sympathetic (SNS) and parasympathetic nervous systems (PNS), are reflected in the low-(LF) and high-frequency (HF) bands in heart rate variability (HRV) while the ratio of the powers in those frequency bands, the so called LF:HF power ratio, has been used to quantify the degree of sympathovagal balance (Sin-Ae Park et al; Int J Environ Res Public Health. 2017 September; 14(9): 1087).

High resolution audio stimuli have proven to enhance relaxation compared to low resolution audio stimuli and showing decrease in Alpha-wave power (T. Harada et al, International Medical Journal (1994) 23(no. 1):1-3⋅April 2016). In comparison to the same results observed in the conducted experiments, the method as described in this disclosure may be considered a novel method to obtain high resolution audio signals, and more specifically, with shape information encoded in the signal, which shows a marked difference in results compared to stereo sound projection using the same high-fidelity audio equipment.

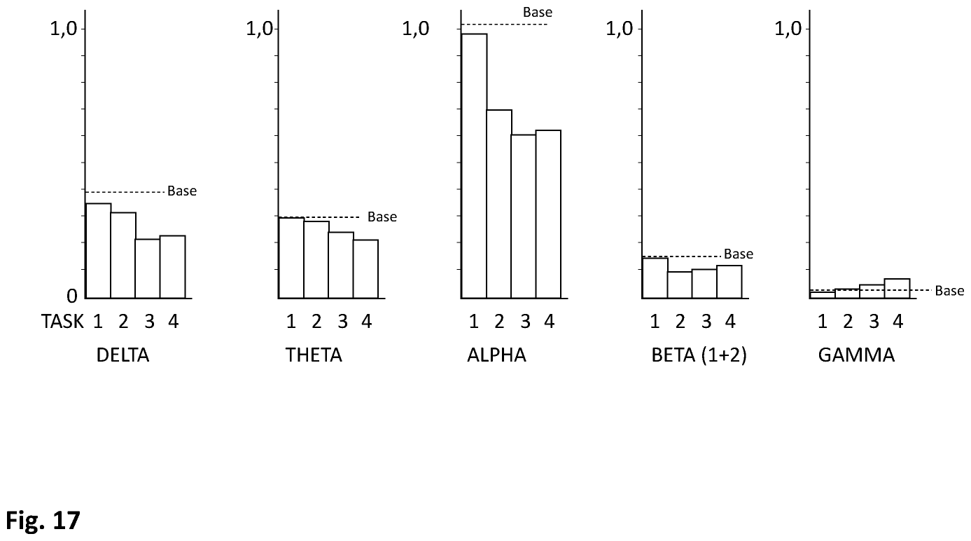

FIG. 17 shows the mean amplitude values of each of the frequency bands (Delta: 1.5-3.5 Hz, Theta: 3.5-7 Hz, Alpha: 8-13 Hz, Beta1: 13-20 Hz, Beta2: 18-25 Hz, Gamma: 30-40 Hz) following 5 minutes of exposure to the aforementioned tasks 1-4. The measurements were taken during the last minute of each epoch. A significant decrease in Alpha-wave activity is observed comparing task 1 ‘stereo sound projection’ to task 2-4 ‘sound shape projection’.

Furthermore, the obtained data suggest that in the Alpha-wave range there is a significant difference between base condition and tasks 2-4. Alpha slightly decreases between base state and stereo sound projection (task 1: N=12 p≤0.061), and then significantly decreases, with variation, between task 1 and sound shape projections (task 2: N=12 p≤0.01, task 3: N=12 p≤0.023, task 4: N=12 p≤0.059). As shown, significant results are obtained between base condition and task 2, and between base condition and task 3; and, to a lesser extent, between base condition and task 4.

A p-value less than 0.05 (typically <0.05) is statistically significant. It indicates strong evidence against the null hypothesis, as there is less than a 5% probability the null is correct and the results are random. (Saul McLeod, https://www.simplypsychology.org/p-value.html, retrieved 22 Jul. 2020).

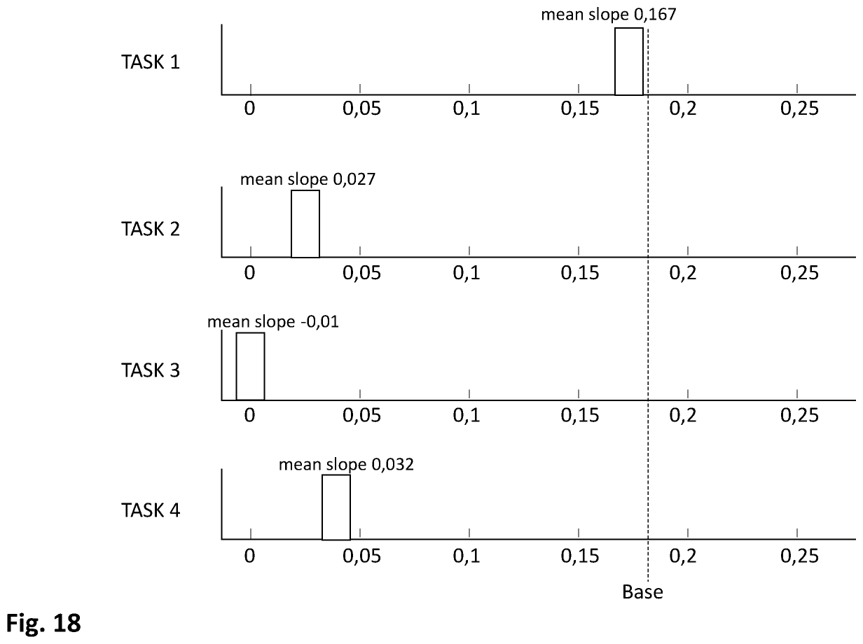

FIG. 18 shows that the Alpha:Beta power ratio is significantly decreased during task 2-4 compared to base state and furthermore significantly decreased between stereo sound projection (task 1) and sound shape projections (task 2-4).

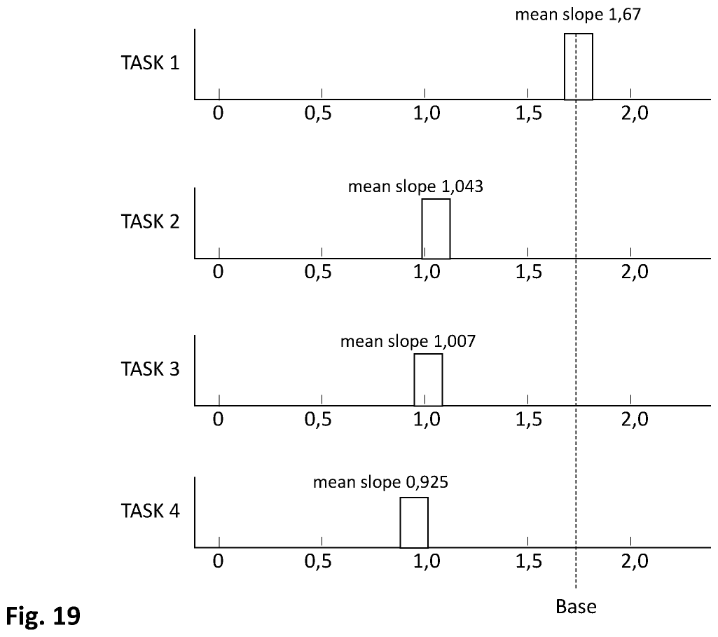

FIG. 19 shows that the LF:HF power ratio of the Heart Rate Variability is significantly decreased during task 2-4 compared to base state and furthermore significantly decreased between stereo sound projection (task 1) and sound shape projections (task 2-4).

The experimental data referred to herein are obtained from a study with the goal to explore whether presentation of sound shape projection, i.e. whether the provision of an audio signal that is configured such that it is perceived by the subject as originating from a virtual sound source having a shape, has an effect on physiological measures (EEG, HRV); and, explore whether different sound shape projections have different effects on physiological measures (EEG, HRV). The study was conducted with a totality of 50 participants N=50 subjects of which 22 female and 28 male subjects. All subjects were healthy young adults between 20-40 years old. Subjects declared not to suffer from any mental or health issues and were not taking any medication regularly. The study was conducted according to the Helsinki Ethics Declaration.

EEG (Electroencephalogram) data was collected by Prof. Dr. Thomas Feiner and Frank Hegger and processed by Dr. Anat Barnea. HRV (Heart Rate Variability) data was collected by Bertram Reinberg. The HRV data was recorded in parallel to EEG recordings. Observations considered for statistics were: the mean amp of frequency bands averaged over all electrodes e=19 (per subject, per band). No spatial localization of the signals was considered besides Left:Right.

The experiment was conducted in a sound-proofed, acoustically treated studio environment with omnidirectional loudspeakers placed above, below and around the subjects as depicted in FIG. 5A. Subjects were instructed to sit still with eyes closed for the entire experimental procedure. All positions of task 1-4 were played on the same loudspeakers, at the same normalized sound pressure levels at the subject and under the same conditions. A sample of the sound stimulus was played in a loop at the same length and volume of pre-recorded high frequency unweighted tuning forks in an interval of an octave (frequency played: #1=272.2 Hz, #2=544.4 Hz).

Subjects were exposed to stereo sound projection (task 1) or a sound shape projection (task 2, 3 or 4). Each sound stimuli was played for an epoch of 5 minutes. All subjects were also monitored for 5 minutes base condition (no sound with eyes closed) and 5 minutes sound stimuli. The presentation of shapes (task 1-4) was randomly intermingled between subjects. The experiment was conducted ‘blind-to-blind’, where both participants and the doctors taking the physiological measurements were unaware of which task was playing and what were the characteristics of the sound samples. Participants were asked to answer assessment questionnaires before and after their exposure to the sound stimuli.

Each condition, including base condition, was measured for 5 minutes of which the last minute was analyzed. Subjects with noisy artifacts were removed from the analysis paradigm, before running statistical analysis.

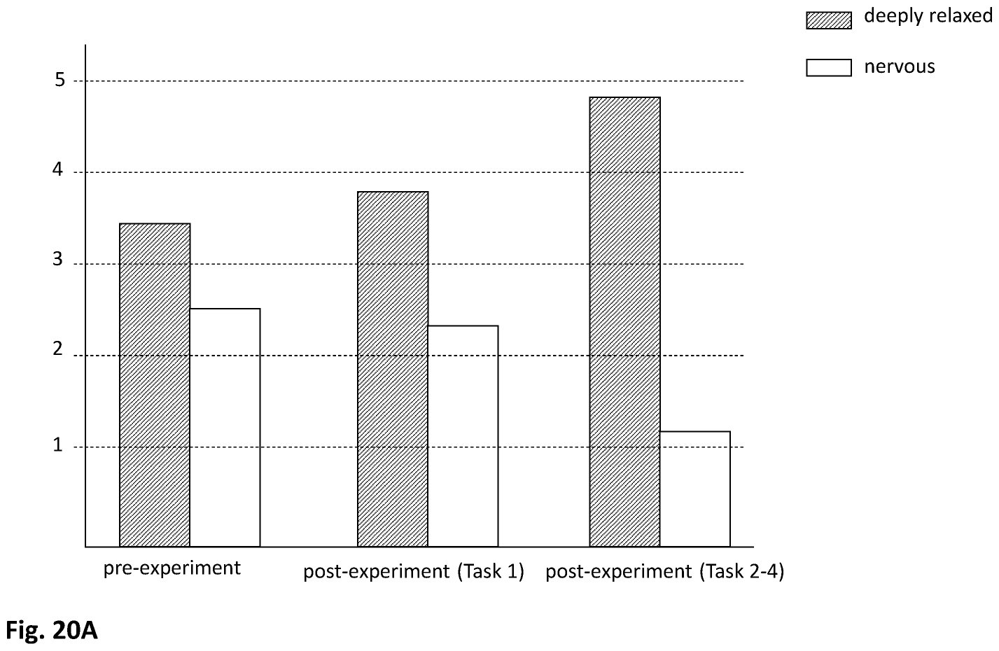

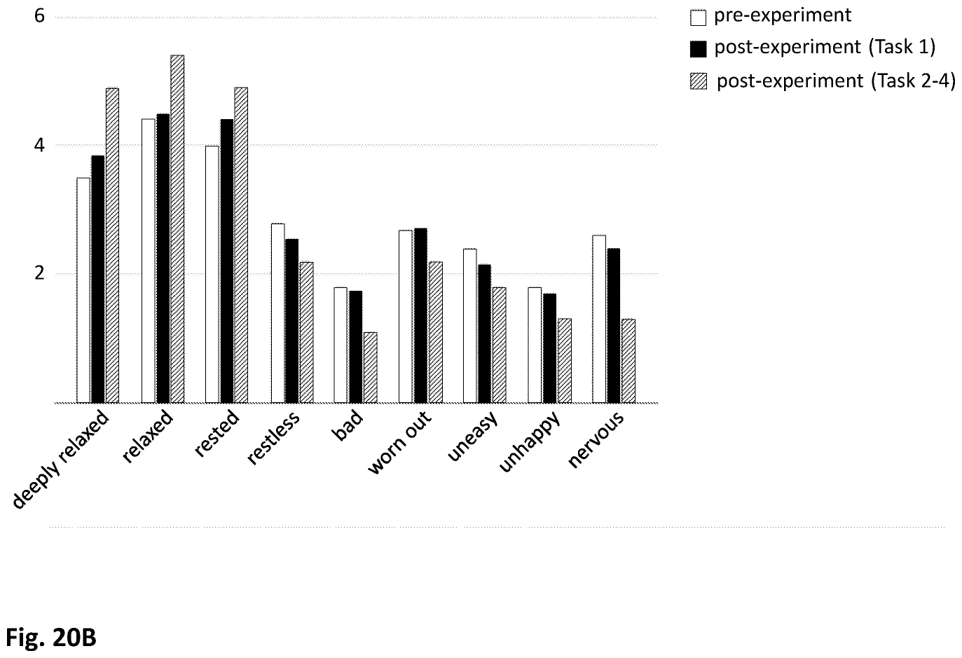

FIG. 20A shows a summary of the results from MDQM answered by the subjects pre- and post-exposure to the sound stimuli. MDMQ is the English version of the German Multidimensional Mood Questionnaire (MDBF) that has proven to be a reliable measure in many researches (https://www.metheval.uni-jena.de/mdbf.php retrieved 22 Jul. 2020). The results show a significant increase in reported deep relaxation of the participants and significant decrease of nervousness, comparing pre- and post-exposure to the projected sound shapes (task 2-4) and comparing the effects post-exposure of stereo sound projection (task 1) to sound shape projection (task 2-4).

FIG. 20B shows a larger report resulting from MDQM questionnaires answered by the subjects pre- and post-exposure to the sound stimuli. All categories show the same tendency in comparison to pre- and post-exposure to the sound stimuli with an increase in all positive feelings measured, and decrease in all negative feelings measured. All categories show statistically significant results for MDMQ questionnaires answered after exposure to task 2-4 (N=36). Rested p≤0.002, Restless p≤0.045, Bad p≤0.001, Worn Out P≤0.030, Uneasy p≤0.025, Relaxed p≤0.001, Unhappy p≤0.048, Nervous p≤0.000, Deeply Relaxed p≤0.001

All participants were required to answer BECK Depression Inventory (BDI) before enrolling in the experiment, the average score of all participants was 7.45 which is considered normal. BECK Depression Inventory (BDI) is used for the concurrent validity of ratings in clinical and nonclinical subjects with regards to the Hamilton Psychiatric Rating Scale for Depression (HRSD). (Aaron T. Beck, Clinical Psychology Review Volume 8, Issue 1, 1988, Pages 77-100).

All participants were requested to answer the Multidimensional Mood Questionnaire (MDMQ) directly before and after each sound stimuli to monitor their well-being and emotional response. Statistical analysis of results from all questionnaires was conducted on SPSS 25.0 using Paired t test method.

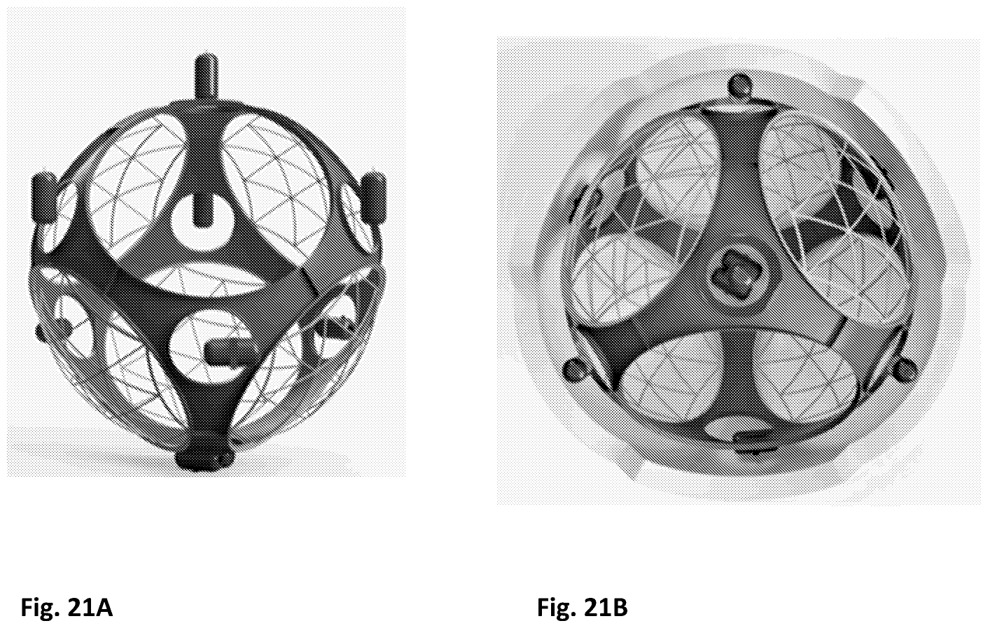

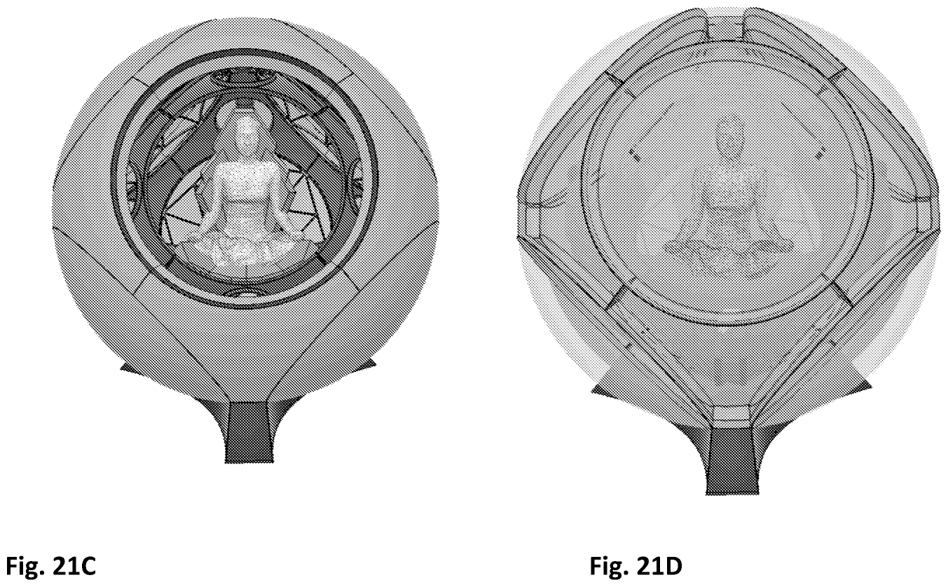

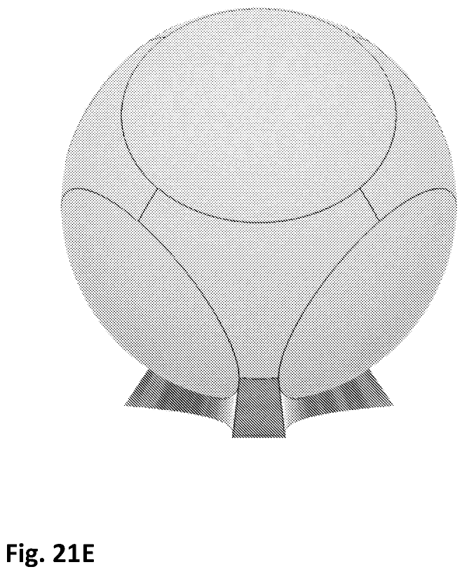

FIG. 21A shows an embodiment of a sound system to improve the physiological condition of a subject comprising a loudspeaker configuration for sound shape projections surrounding an acoustically transparent spherical shell. In an embodiment, omnidirectional loudspeakers are placed at equal radius from a center and equidistant to one another, with right angles from above and below, forming a ‘tilted cube’ or ‘star-tetrahedron’ shape.

FIG. 21B shows an embodiment of the invention, a loudspeaker configuration with on the inside of the sound system's circumference an acoustically transparent spherical shell; and, on the outside of the sound system's circumference a sound-proof shell enclosing the sound system within a spherical pod.

FIG. 21C shows an embodiment of the invention, a sound-proof spherical pod with integrated a loudspeaker configuration shape behind an acoustically transparent inner shell, and a dedicated platform and chair placed in the center of the pod for a subject to sit in its center.

FIG. 21D shows an embodiment of the invention, a see-through of a spherical pod with six outside shells connected by eight corner pieces; eight loudspeakers positioned on the corners of a tilted cube or star-tetrahedron configuration shape within the dimensions of the spherical pod; six inside shells connected by eight corner pieces; and, a platform and chair for a subject to sit in the center of the spherical pod.

FIG. 21E shows an embodiment of the invention, a closed spherical pod to contain a sound system to improve physiological condition of a subject and for a subject to sit in its center.

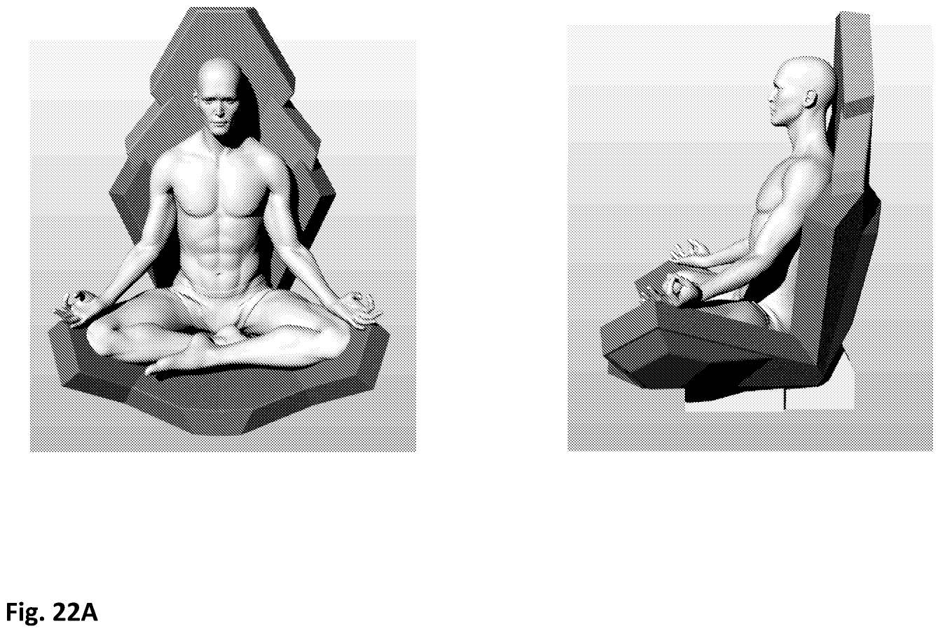

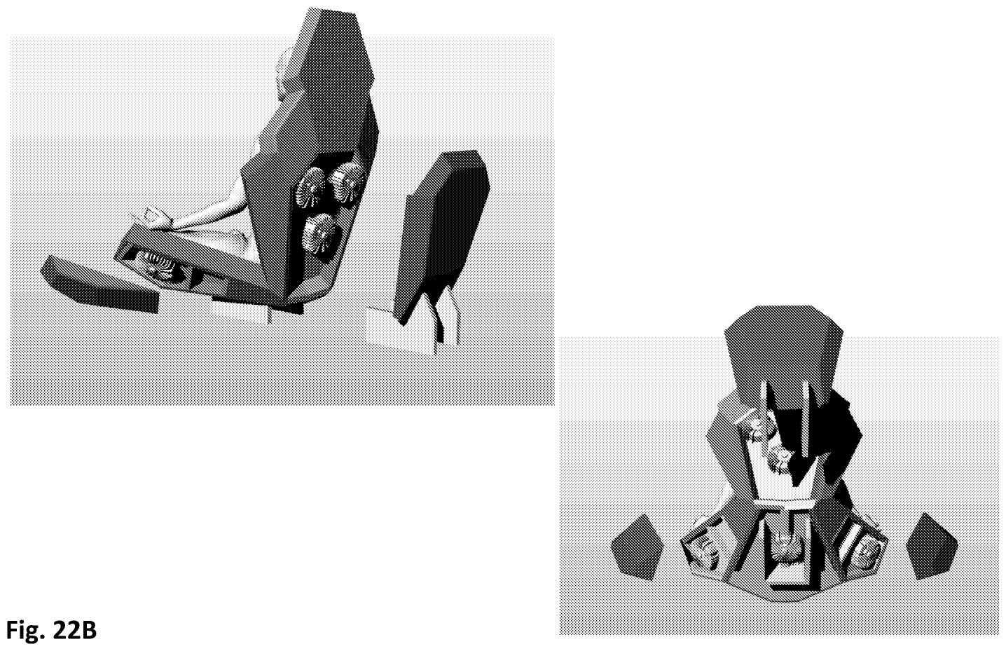

FIG. 22A shows an embodiment of the invention, a subject sitting on a chair supporting a ‘lotus-position’, which may be placed in the center of a spherical pod to contain a sound system to improve physiological condition of a subject, and/or a chair which may contain an integrated loudspeaker configuration for sound shape projection.

FIG. 22B shows an embodiment of the invention, a sound system to improve physiological condition of a subject comprising a loudspeaker configuration for sound shape projections integrated in a chair supporting a subject to sit in a lotus position. Within the seat, back and sides of the chair are positioned integrated loudspeakers and/or vibro-transducers covering the shoulders, back, behind and the knees of a subject sitting in the chair.

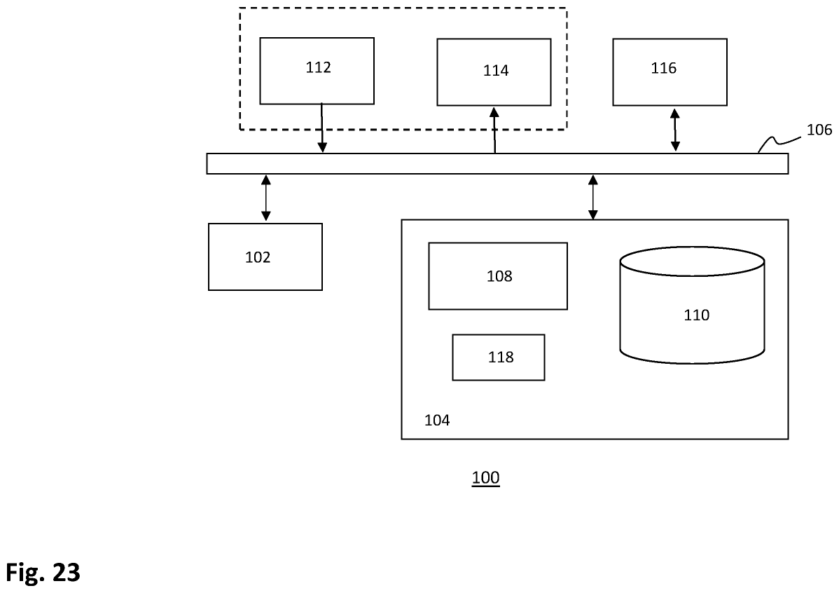

FIG. 23 depicts a block diagram illustrating an exemplary data processing system according to an embodiment.

As shown in FIG. 23 the data processing system 100 may include at least one processor 102 coupled to memory elements 104 through a system bus 106. As such, the data processing system may store program code within memory elements 104. Further, the processor 102 may execute the program code accessed from the memory elements 104 via a system bus 106. In one aspect, the data processing system may be implemented as a computer that is suitable for storing and/or executing program code. It should be appreciated, however, that the data processing system 100 may be implemented in the form of any system including a processor and a memory that is capable of performing the functions described within this specification.

The memory elements 104 may include one or more physical memory devices such as, for example, local memory 108 and one or more bulk storage devices 110. The local memory may refer to random access memory or other non-persistent memory device(s) generally used during actual execution of the program code. A bulk storage device may be implemented as a hard drive or other persistent data storage device. The processing system 100 may also include one or more cache memories (not shown) that provide temporary storage of at least some program code in order to reduce the number of times program code must be retrieved from the bulk storage device 110 during execution.

Input/output (I/O) devices depicted as an input device 112 and an output device 114 optionally can be coupled to the data processing system. Examples of input devices may include, but are not limited to, a keyboard, a pointing device such as a mouse, a touch-sensitive display, or the like. Examples of output devices may include, but are not limited to, a monitor or a display, speakers, or the like. Input and/or output devices may be coupled to the data processing system either directly or through intervening I/O controllers.

In an embodiment, the input and the output devices may be implemented as a combined input/output device (illustrated in FIG. 23 with a dashed line surrounding the input device 112 and the output device 114). An example of such a combined device is a touch sensitive display, also sometimes referred to as a “touch screen display” or simply “touch screen”. In such an embodiment, input to the device may be provided by a movement of a physical object, such as e.g. a stylus or a finger of a user, on or near the touch screen display.

A network adapter 116 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the data processing system 100, and a data transmitter for transmitting data from the data processing system 100 to said systems, devices and/or networks. Modems, cable modems, and Ethernet cards are examples of different types of network adapter that may be used with the data processing system 100.

As pictured in FIG. 23, the memory elements 104 may store an application 118. In various embodiments, the application 118 may be stored in the local memory 108, the one or more bulk storage devices 110, or apart from the local memory and the bulk storage devices. It should be appreciated that the data processing system 100 may further execute an operating system (not shown in FIG. 1) that can facilitate execution of the application 118. The application 118, being implemented in the form of executable program code, can be executed by the data processing system 100, e.g., by the processor 102. Responsive to executing the application, the data processing system 100 may be configured to perform one or more operations or method steps described herein.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 102 described herein.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of embodiments of the present invention has been presented for purposes of illustration, but is not intended to be exhaustive or limited to the implementations in the form disclosed. Many modifications and variations will be apparent to those of ordinary skill in the art without departing from the scope and spirit of the present invention. The embodiments were chosen and described in order to best explain the principles and some practical applications of the present invention, and to enable others of ordinary skill in the art to understand the present invention for various embodiments with various modifications as are suited to the particular use contemplated.

The inventors acknowledge dr. Claire Glanois and dr. Galit Fuhrmann Alpert for their contributions to this disclosure.

1. A method for improving a physiological condition of a subject, e.g. a human or animal, the method comprising:
providing an audio signal to the subject, wherein the audio signal is associated with a virtual sound source having a shape and a position relative to the subject, wherein
the virtual sound source is defined by a plurality of virtual points, each virtual point having a position relative to the subject, and wherein
the audio signal comprises audio signal components for the respective virtual points of the virtual sound source, wherein each audio signal component has been determined based on the position of its associated virtual point such that the audio signal is perceived by the subject as originating from the virtual sound source having said shape and said position relative to the subject.

2. The method according to claim 1, wherein the method is a non-therapeutic method.

3. The method according to claim 1, wherein the audio signal is obtained by
obtaining virtual sound source information defining the respective positions of the virtual points relative to the subject, the virtual points defining the virtual sound source having said shape and said position relative to the subject,
obtaining an input audio signal, and
determining the respective audio signal components for the respective virtual points based on the input audio signal and based on the respective positions of the virtual points, wherein
for each audio signal component respectively associated with a virtual point, determining the audio signal component comprises:
modifying the input audio signal to obtain a modified audio signal component using a signal delay operation introducing a time delay, wherein the time delay is based on the position of the virtual point associated with the audio signal component relative to the shape of the virtual sound source; and
determining the audio signal component based on a combination of the input audio signal, or of an inverted and/or attenuated or amplified version of the input audio signal, and the modified audio signal component; and
combining the determined audio signal components to obtain the audio signal.

4. The method according to claim 3, wherein the input audio signal is an audio signal produced by a tuning fork.

5. The method according to claim 1, comprising:
providing the audio signal to the subject using a plurality of loudspeakers, and
determining a loudspeaker audio signal for each loudspeaker, wherein each loudspeaker audio signal is determined based on the audio signal components, and
providing the loudspeaker audio signals to the respective loudspeakers.

6. The method according to claim 5, wherein
determining the loudspeaker audio signal for each loudspeaker comprises, for each loudspeaker audio signal, attenuating each audio signal component based on a loudspeaker specific coefficient in order to obtain a loudspeaker specific set of attenuated audio signal components and combining the attenuated audio signal components in the loudspeaker specific set of attenuated audio signal components.

7. The method according to claim 5, wherein the plurality of loudspeakers comprises a loudspeaker in front of the subject and a loudspeaker behind the subject and a loudspeaker to the right of the subject and a loudspeaker to the left of the subject and a loudspeaker above the subject and a loudspeaker below the subject.

8. The method according to claim 5, wherein the plurality of loudspeakers comprises at least eight loudspeakers:
a loudspeaker above the subject;
a loudspeaker in front of, below the subject;
a loudspeaker in front of, to the left of, above the subject;
a loudspeaker in front of, to the right of, above the subject;
a loudspeaker behind, above the subject;
a loudspeaker behind, to the left of, below the subject;
a loudspeaker behind, to the right of, below the subject; and
a loudspeaker below the subject.

9. The method according to claim 1, wherein the virtual sound source is shaped as a cube or a pyramid or a sphere.

10. The method according to claim 1, wherein the audio signal is configured such that it is perceived by the subject that said virtual sound source is surrounding the subject.

11. The method according to claim 1, comprising providing the audio signal to the subject using a plurality of loudspeakers that surround the subject.

12. The method according to claim 1, wherein the audio signal is provided to the subject for at least one minute.

13. The method according to claim 1, wherein the virtual sound source associated with the audio signal changes shape and/or position while the audio signal is provided to the subject thus wherein the respective positions relative to the subject of the respective virtual points defining the virtual sound source change while the audio signal is provided to the subject such that the audio signal is perceived by the subject as originating from the virtual sound source having a varying position and/or orientation relative to the subject.

14. The method according to claim 1, wherein one or more virtual points of the virtual sound source are virtually positioned at a depth below the subject, wherein the audio signal is obtained by
for each audio signal component associated with a virtual point that is positioned at a virtual depth below the subject, adding depth characteristics to the audio signal component in question comprising modifying the audio signal component in question using a time delay operation introducing a time delay, a signal attenuation and a signal feedback operation in order to obtain a modified version of the audio signal component and combining the modified version of the audio signal component with the audio signal component in question, wherein
the signal attenuation is performed in dependence of the virtual depth below the subject of the virtual point associated with the audio signal component in question.

15. The method according to claim 1, wherein one or more virtual points of the virtual sound source are virtually positioned at a height above the subject, wherein the audio signal is obtained by
for each audio signal component associated with a virtual point that is positioned at a virtual height above the subject, adding height characteristics to the audio signal component in question comprising modifying the audio signal component in question using a signal inverting operation, a signal delay operation introducing a time delay and a signal attenuation to obtain a modified version of the audio signal component and combining the modified version of the audio signal component with the audio signal component in question, wherein
the signal attenuation is performed in dependence of the virtual height of the virtual sound source.

16. The method according to claim 1, wherein one or more virtual points of the virtual sound source are virtually positioned at a virtual distance from the subject, wherein the audio signal is obtained by
for each audio signal component associated with a virtual point that is positioned at a virtual distance from the subject, adding distance characteristics to the audio signal component in question comprising modifying the audio signal component in question using a first signal delay operation introducing a first time delay, a first signal attenuation operation and a signal feedback operation in order to obtain a first modified version of the audio signal component and combining the first modified version of the audio signal component with the audio signal component in question to obtain a second modified version of the audio signal component and performing a second signal attenuation, wherein
the first and second signal attenuation are performed in dependence of the virtual distance from the subject.

17. A system for improving a physiological condition of a subject, e.g. a human or animal, the system comprising:
a data processing system for determining an audio signal associated with a virtual sound source having a shape and a position relative to the subject, wherein
the virtual sound source is defined by a plurality of virtual points, each virtual point having a position relative to the subject, and wherein
the audio signal comprises audio signal components for the respective virtual points of the virtual sound source, the data processing system being configured to
determine each audio signal component based on the position of its associated virtual point such that the audio signal is perceived by the subject as originating from the virtual sound source having said shape and said position relative to the subject, and the system comprising
one or more loudspeakers for providing the determined audio signal to the subject.

18. A computer readable medium comprising instructions which when executed by a processor executes a method comprising:
providing an audio signal to a subject, wherein the audio signal is associated with a virtual sound source having a shape and a position relative to the subject, wherein
the virtual sound source is defined by a plurality of virtual points, each virtual point having a position relative to the subject, and wherein
the audio signal comprises audio signal components for the respective virtual points of the virtual sound source, wherein each audio signal component has been determined based on the virtual position of its associated virtual point such that the audio signal is perceived by the subject as originating from the virtual sound source having said shape and said position relative to the subject.

19. The method of claim 16 and further comprising performing a second signal delay operation introducing a second time delay on the second modified version of the audio signal component.
